(19) United States
(12) Patent Application Publication
(10) Pub. No.: US 2026/0130763 A1
(43) Pub. Date: May 14, 2026

(54) HEART VALVE REPAIR DEVICES AND DELIVERY DEVICES THEREFOR

(71) Applicant: Edwards Lifesciences Corporation, Irvine, CA (US)

(72) Inventors: Sergio Delgado, Irvine, CA (US); Samuel Jacob Kipperman, Boulder, CO (US); Christopher Rene Williams, Orange, CA (US); Zoe Eloise Anderson, Seattle, WA (US); Pablo Hernan Catania, Costa Mesa, CA (US); Robert Turner Bradshaw, Irvine, CA (US); Eric Robert Dixon, Villa Park, CA (US); Rhayoung Kwon, Irvine, CA (US); Asim Minhaj Syed, Irvine, CA (US); Alex Raymond Diehl, Lake Forest, CA (US); Sydney Marie Tighe, Mission Viejo, CA (US); Joseph Chi-Ho Lau, Tustin, CA (US); Raffi Sempad Pinedjian, Fountain Valley, CA (US); Jensen Chen, Walnut, CA (US); Yong Chul Lee, Anaheim, CA (US); Robert Francisco Gomez, Temecula, CA (US); Amy Tsai, Rancho Santa Margarita, CA (US); Henry Thek Chan, Trabuco Canyon, CA (US); Ismail Thazhapilavil, Irvine, CA (US)

(21) Appl. No.: 19/430,117

(22) Filed: Dec. 22, 2025

Related U.S. Application Data
(63) Continuation of application No. PCT/US2024/038555, filed on Jul. 18, 2024.
(60) Provisional application No. 63/514,785, filed on Jul. 20, 2023.

(51) Int. Cl. A61F 2/24; A61M 25/00; A61M 25/01; A61M 25/06

(52) U.S. Cl. CPC A61F 2/246; A61F 2/2466; A61M 25/005; A61M 25/0136; A61M 2025/0681

(57) ABSTRACT

A catheter assembly includes a handle assembly, a steerable catheter, and an actuation element. The handle assembly includes a handle housing, a guide member, a follower, and a driver. The driver engages the follower to cause the follower to move relative to the guide member. The actuation element is connected to the follower and the steerable catheter. Movement of the follower relative to the guide member causes the actuation element to engage the steerable catheter to move the steerable catheter between the flexed configuration and the unflexed configuration.

### RELATED APPLICATIONS

The present application is a continuation of Patent Cooperation Treaty Application No. PCT/US2024/038555, filed on Jul. 18, 2024, titled “HEART VALVE REPAIR DEVICES AND DELIVERY DEVICES THEREFOR,” which claims the benefit of U.S. Provisional Patent Application No. 63/514,785, filed on Jul. 20, 2023, titled “HEART VALVE REPAIR DEVICES AND DELIVERY DEVICES THEREFOR,” which are incorporated herein by reference in their entireties.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D” shaped, oval, or otherwise out-of-round cross-sectional shape having major and minor axes. The anterior leaflet may be larger than the posterior leaflet, forming a generally “C” shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to limit the scope of the disclosed subject matter in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

Devices for repairing and/or treating a native valve of a patient are disclosed. The devices can be valve repair devices, implantable devices, valve treatment devices, implants, etc. While sometimes described as an implantable device for illustration purposes in various examples herein, similar configurations can be used on other devices, e.g., valve repair devices, treatment devices, etc., that are not necessarily implanted and may be removed after treatment.

In some implementations, there is provided a device (e.g., treatment device, repair device, implantable device, implant, etc.) that is configured to be positioned within a native heart valve to allow the native heart valve to form a more effective seal. In some implementations, the device is part of a system (e.g., a valve repair system, valve treatment system, etc.) including a delivery system having a catheter and a control handle wherein the device is coupled to the delivery system.

In some implementations, a device (e.g., treatment device, repair device, implantable device, implant, etc.) includes an anchor portion. Each anchor includes a plurality of paddles that are each moveable between an open position and a closed position.

In some implementations, a device (e.g., treatment device, repair device, implantable device, implant, etc.) includes a coaptation element, a first anchor assembly configured to grasp a first leaflet of a native heart valve, and a second anchor assembly configured to grasp a second leaflet of the native heart valve. In some implementations, the first anchor assembly is connected to a first side of the coaptation element, and the second anchor assembly is connected to a second side of the coaptation element.

In some implementations, there is provided a catheter assembly for a transvascular delivery system. In some implementations, the catheter assembly includes a handle housing and a sheath extending longitudinally from the handle housing. In some implementations, the sheath includes a coil that has one or more supporting elements and one or more tubes that are coiled to define a main lumen, where each of the one or more tubes defines a lumen.

In some implementations, the lumen of each of the one or more tubes is configured to receive a control element such that the control element can be moved in a direction from a proximal portion of the sheath to a distal portion of the sheath and/or vice versa.

In some implementations, each tube of the one or more tubes comprises a hypotube.

In some implementations, the hypotube is made from nitinol.

In some implementations, the one or more tubes of the coil include at least two tubes.

In some implementations, at least a portion of the coil includes the one or more supporting elements and the one or more tubes being coiled in a clockwise direction.

In some implementations, at least a portion of the coil has a pitch angle of between about 2 degrees and about eighty degrees.

In some implementations, another portion of the coil includes the one or more supporting elements and the one or more tubes being in a straight configuration.

In some implementations, the sheath further includes an outer coil layer that is positioned over the coil, where the outer coil layer has a plurality of supporting elements.

In some implementations, the plurality of supporting elements of the outer coil layer are coiled in a counterclockwise direction and at least a portion of the coil includes the one or more supporting elements and the one or more tubes being coiled in a clockwise direction.

In some implementations, the control element includes a wire.

In some implementations, the catheter assembly includes a handle assembly that includes the handle housing, a guide member, a follower that is movably connected to the guide member, and a driver that is engageable by a user to move the follower relative to the guide member.

In some implementations, the handle housing includes at least one window that allows for external threads of the follower to be engaged by internal threads of the driver such that the driver can move the follower relative to the guide member.

In some implementations, the follower includes an indicator, and the handle housing includes an indicator window that allows for the indicator of the follower to be visible to a user during use of the catheter assembly.

In some implementations, the handle assembly further includes an actuation member that is attached to the driver such that a user can engage the actuation member to cause the driver to engage the follower and move the follower relative to the guide member.

In some implementations, the guide member has a hexagonal shape.

In some implementations, the handle housing has one or more walls that are shaped to engage with the hexagonal shape of the guide member to prevent the guide member from rotating relative to the housing.

In some implementations, the handle housing includes two or more components that are configured to be connected together to create the handle housing.

In some implementations, the guide member includes two or more components that are configured to be connected together to create the guide member.

In some implementations, the catheter assembly further includes an actuation element that is connected to the sheath and the follower of the handle assembly such that movement of the follower relative to the guide member of the handle assembly causes the actuation element to engage the sheath and move the sheath between a flexed configuration and an unflexed flexed configuration.

In some implementations the handle assembly further includes a connector that attaches the actuation element to the follower.

In some implementations, the actuation element is attached to the connector by a stitched connection.

In some implementations, the connector includes a strain relief portion and a connection portion, where the strain relief portion has one or more strain relief members for engaging the actuation element to reduce strain applied to the actuation element as a force is applied to the actuation element during use of the catheter assembly, and where the actuation element is connected to the connector by the stitched configuration.

In some implementations, there is provided a catheter assembly for a transvascular delivery system. In some implementations, the catheter assembly includes a handle assembly, a steerable catheter, and an actuation element.

In some implementations, the handle assembly includes a handle housing, a guide member at least partially disposed within the handle housing, a follower movably attached to the guide member, and a driver that is configured to engage the follower to cause the follower to move relative to the guide member.

In some implementations, the steerable catheter extends from the handle assembly and is movable between a flexed configuration and an unflexed configuration.

In some implementations, the actuation element is connected to the follower of the handle assembly and the steerable catheter.

In some implementations, movement of the follower relative to the guide member causes the actuation element to engage the steerable catheter to move the steerable catheter between the flexed and unflexed configurations.

In some implementations the handle assembly includes two or more components that are configured to be connected to create the handled housing.

In some implementations, the guide member includes two or more components that are configured to be connected to create the guide member.

In some implementations, the follower has a body member and threads extending outward from the body member, where the threads are configured to be engaged by threads of the driver to move the follower relative to the guide member.

In some implementations, the handle housing has at least one window that allows for the threads of the follower to be engaged by threads of the driver such that the driver can move the follower relative to the guide member.

In some implementations, the follower includes an indicator, and the housing includes an indicator window that allows for the indicator of the follower to be visible to a user during use of the catheter assembly.

In some implementations, the handle assembly further includes an actuation member that is attached to the driver such that a user can engage the actuation member to cause the driver to engage the follower and move the follower relative to the guide member.

In some implementations, the guide member has a hexagonal shape.

In some implementations, the handle housing has one or more walls that are shaped to engage the hexagonal shape of the guide member to prevent the guide member from rotating relative to the housing.

In some implementations, the actuation element includes a wire.

In some implementations, the handle assembly further includes a connector that attaches the actuation element to the follower.

In some implementations, the actuation element is attached to the connector by a stitched connection.

In some implementations, the connector includes a strain relief portion and a connection portion, where the strain relief portion has one or more strain relief members for engaging the actuation element during use of the catheter assembly, and where the connection portion is configured to receive the actuation element such that the actuation element is connected to the connector by the stitched connection.

In some implementations, each of the strain relief members has a curved portion for engaging with the actuation element.

In some implementations, at least one of the strain relief members has a circular shape.

In some implementations, the strain relief portion includes a first opening disposed between a first strain relief member and a second strain relief member and a second opening disposed between the second strain relief member and a third strain relief member.

In some implementations, the actuation element engages the first strain relief member, extends through the first opening, engages the second strain relief member, extends through the second opening, and engages the third strain relief member when the actuation element is attached to the connection portion of the connector.

In some implementations, the connector has one or more openings for connecting the actuation element to the connector by the stitched connection.

In some implementations, the connector includes a support member proximate the one or more openings for engaging the actuation element and reducing the strain placed on the actuation element due to contact with the connector at the one or more openings.

In some implementations, the connector is made from a first material and the support member is made from a second material that is different than the first material.

In some implementations, the connector is made of a metal material and the support member is made of a plastic material.

In some implementations, the catheter assembly further includes a seal assembly having a seal housing, a seal member positioned within the seal housing, and a cap configured to be attached to the seal housing.

In some implementations, the cap has a plurality of connection members for attaching the cap to the seal housing.

In some implementations, the connection members of the cap are configured to attach the cap to the seal housing by a snap connection.

In some implementations, the seal housing has one or more alignment members, and the cap has one or more alignment slots for receiving the alignment members of the seal housing.

In some implementations, the one or more alignment members are alignment ribs.

In some implementations, the seal housing includes three alignment members, and the cap includes six alignment slots.

In some implementations the cap includes a cutout configured to assist a user in aligning the cap with the seal housing.

In some implementations, the cutout has a hexagonal shape.

In some implementations, the seal housing is integral to the guide member.

In some implementations, the catheter assembly further includes a catheter coupler. In some implementations, the catheter coupler includes a catheter connection lumen connected to the steerable catheter, an outer circumferential passage, a plurality of connecting passages the connect the catheter connection lumen to the outer circumferential passage, and an outlet portion in fluid communication with the outer circumferential passage.

In some implementations, the connecting passages are offset from a center line of the catheter connection lumen.

In some implementations, the catheter assembly further includes a connector for attaching the actuation element to the follower of the handle assembly, where the connector is movably attached to the follower.

In some implementations, the connector is configured to move distally relative to the follower when a user engages the drive to move the steerable catheter from the flexed configuration to the unflexed configuration while a shape of a lumen of the patient in which the steerable catheter is disposed causes the steerable catheter to maintain the flexed configuration.

In some implementations, there is provided a connector for attaching an actuation element to a handle assembly of a catheter assembly. In some implementations, the connector includes a connector body having a strain relief portion and a connection portion.

In some implementations, the strain relief portion includes a first strain relief member, a second strain relief member, a third strain relief member, a first opening extending through the connector body and disposed between the first and second strain relief members, and/or a second opening extending through the connector body and disposed between the second and third strain relief members.

In some implementations, the connection portion includes two or more openings for receiving the actuation element such that the actuation element is attached to the connector body by a stitched connection.

In some implementations, the strain relief portion has one or more alignment walls for maintaining the actuation wire within the strain relief portion.

In some implementations, the first, second, and third strain relief members include a curved portion for engagement with the actuation element.

In some implementations, at least one of the first, second, and third strain relief members has a circular shape.

In some implementations, two or more openings of the connection portion include at least three openings for receiving the actuation element for attaching the actuation element to the connector body by the stitched connection.

In some implementations, the connection portion has two or more second openings for receiving a second actuation element such that the second actuation element is connected to the connector body by a stitched connection.

In some implementations, the connector body is made of a metal material.

In some implementations, there is provided a seal assembly for a catheter assembly, the seal assembly including a seal housing, a seal member disposed within the seal housing, and a cap configured to attach to the seal housing.

In some implementations, the seal housing has one or more alignment members, and the cap has one or more alignment slots that are configured to receive the one or more alignment members of the seal housing.

In some implementations, the seal housing has three alignment members, and the cap has size alignment slots, where each alignment member is positioned 120 degrees from an adjacent alignment member, and where each alignment slot is positioned 60 degrees from an adjacent alignment slot.

In some implementations, the cap includes an alignment cut out that is configured for assisting a user in connecting the cap to the seal housing.

In some implementations, the alignment cutout has a hexagonal shape.

In some implementations, the cap has a plurality of connection elements that are configured to attach the cap to the seal housing by a snap connection.

In some implementations, there is provided a catheter coupler that includes a housing, and one or more seals. In some implementations, the housing has a catheter connection lumen, a plurality of passages, and an outer circumferential channel. In some implementations, each passage of the plurality of passages is offset from a center line of the catheter connection lumen.

In some implementations, the cap is rotatably coupled to the housing, and the cap has an outlet port in fluid communication with the outer circumferential passage. In some implementations, the one or more seals provide seals between the housing and the cap that direct fluid flow from the outer circumferential passage to the outlet port.

In some implementations, the cap is rotatable to position the outlet port in a vertical orientation without rotating the housing.

In some implementations, there is provided a catheter coupler that includes a catheter connection lumen, an outer circumferential passage, a plurality of connecting passages that each connect the catheter connection lumen to the outer circumferential passage, and an outlet port in fluid communication with the outer circumferential passage.

In some implementations, each connecting passage of the plurality of connecting passages is offset from a center line of the catheter connection lumen.

In some implementations, the outer circumferential passage and the plurality of passages are sized such that when the outlet port is oriented in a downwardly facing direction, upper ones of the connecting passages contain air, lower ones of the connecting passages contain liquid, and the air is drawn out of the outlet port.

In some implementations, a catheter assembly for a transvascular deliver system is provided including: a patterned tube disposed between an inner and an outer liners, the patterned tube including a tube wall having at least first and second pattern openings; the first pattern openings including an interrupted spiral pattern; and the second pattern openings including a plurality of flex windows and a plurality of reflow openings disposed opposite the flex windows; the plurality of flex windows including a transition region having flex windows of different size.

In some implementations, the plurality of flex windows includes flex windows of the same size.

In some implementations, the transition region is disposed proximate the plurality of flex windows having the same size.

In some implementations, the plurality of flex windows includes a flex region length and the transition region includes approximately half the flex region length.

In some implementations, the flex windows of different size includes at least a first flex window having a first size, a second flex window having a second size that is larger than the first size, and a third flex window having a third size that is larger than the second size.

In some implementations, the flex windows of different size include a plurality of flex windows having sizes that progressively increase or decrease.

In some implementations, the flex windows of different size include a plurality of flex windows having sizes that progressively increase at a rate of approximately 110 to 120 percent from window to window.

In some implementations, the flex windows of different size include a plurality of flex windows having sizes that progressively decrease at a rate of approximately 80 to 90 percent from window to window.

In some implementations, the patterned tube further includes a pull ring having a plurality of projecting members, each projecting member including a first and second tabs separated by a compressible gap.

In some implementations, the patterned tube further includes a distal end portion having a plurality of recesses configured for an interference fit connection.

In some implementations, a connector for a catheter system is provided including: a body having a base portion, a plurality of projections extending from the base portion, and a plurality of gaps; each projection including a neck portion and a head portion larger than the neck portion, the neck portion located between the base portion and the head portion; and each gap located between at least two projections.

In some implementations, the at least one neck and head portion form a cleat.

In some implementations, the neck portion includes curved walls.

In some implementations, the neck portion includes a tapered center section.

In some implementations, the neck portion includes first and second end sections and a third section between the first and second sections, and wherein the third section is smaller than the first and section sections.

In some implementations, the head portion includes at least one curved side wall.

In some implementations, the head portion includes a body having a slot therein.

In some implementations, the head portion includes a body having first and second portions.

In some implementations, the plurality of projections include at least one projection having a different size than the other projections.

In some implementations, the plurality of projections include at least two projections having the same size.

In some implementations, the base portion includes first and second walls separated by a gap.

In some implementations, the base portion includes at least one opening proximate at least one projection.

In some implementations, the base portion includes at least one extension longitudinally extending from the base portion.

In some implementations, the base portion includes a slot longitudinally extending from the base portion.

In some implementations, at least one actuation element is provided having a counterclockwise winding arrangement around the plurality of projections.

In some implementations, at least one actuation element is provided having a clockwise winding arrangement around the plurality of projections.

In some implementations, at least one actuation element is provided having a cross-over winding arrangement around at least one projection.

In some implementations, at least one actuation element is provided having a counterclockwise winding arrangement around at least one projection and a clockwise winding arrangement around at least another projection.

In some implementations, at least one actuation element is provided having a figure eight winding arrangement around at least two projections.

In some implementations, at least one actuation element is provided having a looped winding arrangement around the plurality of projections.

In some implementations a bridge extending between two head portions.

In some implementations two extensions extending from the two head portions.

In some implementations, a connector assembly for a catheter system includes an actuation element, a first clamp half, and a second clamp half. The actuation element is configured to flex a distal catheter end. The actuation element is clamped between the first clamp half and the second clamp half.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of examples in the present disclosure, a more particular description of certain examples and implementations will be made by reference to various aspects of the appended drawings. These drawings depict only some implementations of the present disclosure and are therefore not to be considered limiting of the scope of the disclosure. Moreover, while the figures can be drawn to scale for some examples, the figures are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing valve regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example of an example device or implant, in various stages of deployment;

FIG. 15 shows an example of an example device or implant that is similar to the device illustrated by FIGS. 8-14, but where the paddles are independently controllable;

FIGS. 16-21 show the example device or implant of FIGS. 8-14 being delivered and deployed within a native valve;

FIG. 22 shows a perspective view of an example device or implant in a closed position;

FIG. 23 shows a perspective view of an example device or implant in a closed position;

FIG. 24 illustrates an example valve repair and/or treatment device with paddles in an open position;

FIG. 25A illustrates an example valve repair and/or treatment device with paddles in a closed position;

FIG. 25B illustrates a top view of an example valve repair device;

FIG. 26 illustrates a perspective view of an example device having paddles of adjustable widths;

FIG. 27 is a cross-section of the example device of FIG. 26 in which the device is bisected;

FIG. 28 is another cross-section of the example device of FIG. 26 in which the device is bisected along a plane perpendicular to the plane illustrated in FIG. 28;

FIG. 29 is a schematic illustration of an example catheter assembly coupled to an example device in which an actuation element is coupled to a paddle actuation control and to a driver head of the device;

FIG. 30 is an illustration of the assembly of FIG. 29 with the example device rotated 90 degrees to show the paddle width adjustment element coupled to an inner end of the connector of the device and coupled to a paddle width control;

FIG. 31 illustrates a distal end of an example system or assembly including an example delivery system and an example device;

FIG. 32 illustrates a proximal end of the example system or assembly of FIG. 31;

FIG. 33 illustrates an example catheter assembly for use in a delivery system coupled to a device or implant;

FIG. 34 illustrates a schematic illustration of an example catheter assembly coupled to an example device, in which each of the clasp actuation lines is coupled to a clasp control member positioned on the handle and the actuation element is coupled to a control element positioned on the handle;

FIG. 35 illustrates a sheath of a steerable catheter assembly having a stiffened length between a proximal end of the steerable catheter sheath and a steerable portion;

FIG. 36 illustrates a steerable catheter assembly having two hypotubes having different stiffnesses;

FIG. 37 illustrates a steerable catheter assembly having a laser-cut hypotube positioned over a braid, mesh, or woven material to increase the stiffness of a portion of the steerable catheter assembly;

FIG. 38 illustrates four segments of a laser-cut hypotube;

FIG. 39 illustrates a hypotube having an interrupted spiral cut;

FIGS. 40A and 40B are radial cross-sections of example multi-layer sheaths;

FIG. 41A is a longitudinal cross-section of an example multi-layer sheath;

FIG. 41B is a longitudinal cross-section of a proximal portion of the multi-layer sheath of FIG. 41A;

FIG. 42 illustrates an example catheter assembly with portions removed to illustrate internal components;

FIG. 43 illustrates an example catheter assembly with portions removed to illustrate internal components;

FIGS. 44A, 44B, and 44C illustrate example braid patterns having a tube for accommodating a control element woven therein;

FIG. 44D is a longitudinal cross-section of an example multi-layer sheath incorporating the braid of any one of FIGS. 44A-44C;

FIG. 44E illustrates an example braid pattern having a tube for accommodating a control element woven therein;

FIG. 44F is a longitudinal cross-section of an example multi-layer sheath incorporating the braid of FIG. 44E;

FIG. 45 illustrates a perspective view of a coil for a catheter assembly, where the coil has one or more tubes incorporated therein;

FIG. 46 illustrates a partial perspective view of the coil of FIG. 45 showing the portion of the coil labeled by box A in FIG. 45;

FIG. 47 illustrates another perspective view of the coil of FIG. 45;

FIG. 48 illustrates a partial perspective view of the coil of FIG. 47, showing the portion of the coil labeled by box B in FIG. 47;

FIG. 49 illustrates an example multi-layer sheath for a catheter assembly that incorporates the coil of FIG. 45;

FIG. 50 illustrates a cross-section of the multi-layer sheath of FIG. 49 taken along the line 50-50 shown in FIG. 49;

FIG. 51 illustrates a perspective view of an example of a multi-layer sheath for a catheter assembly that incorporates a coil;

FIG. 52 illustrates a partial perspective view of the multi-layer sheath of FIG. 51;

FIG. 53 illustrates an example of a multi-layer sheath system;

FIG. 54 is a cross-sectional view of a patterned hypotube and a liner of the multi-layer sheath system of FIG. 53;

FIG. 55 is a partial perspective view of a distal end portion of a patterned hypotube, a liner, and an outer layer shown as transparent of the multi-layer sheath system of FIG. 53;

FIG. 56 illustrates a schematic view of an example of a patterned hypotube;

FIG. 57 illustrates an example of a patterned hypotube distal end portion;

FIG. 58 illustrates an example of a patterned hypotube;

FIG. 59 illustrates an example of a patterned hypotube;

FIG. 60 is a partial perspective view of a hypotube in an unbent or unflexed state or configuration;

FIG. 61 is a partial perspective view of a hypotube in a bent or flexed state or configuration;

FIG. 62 is a partial perspective view of a hypotube in a second bent or flexed state or configuration;

FIG. 63 is a perspective view of an example of a pull ring element;

FIG. 64 is a magnified partial view of an example of an arrangement for connecting a pull ring element to a hypotube;

FIG. 65 is a partial perspective view of an example of a multi-lumen liner of a multi-layer sheath system;

FIG. 66 is a perspective view of an example of a proximal end portion of a hypotube of a multi-layer sheath system;

FIG. 67 is a partial perspective view of the proximal end portion of the laser cut hypotube of FIG. 66 with a jacket disposed over the hypotube;

FIG. 68 illustrates an example handle assembly for a steerable catheter assembly;

FIG. 69 illustrates an exploded view of the handle assembly of FIG. 68;

FIG. 70 illustrates a partial exploded view of the handle assembly of FIG. 68;

FIG. 71 illustrates an example guide member for the handle assembly of FIG. 68 with a steerable catheter attached thereto;

FIG. 72 illustrates a partial view of the handle assembly of FIG. 68 with portions removed to illustrate internal components thereof;

FIG. 73 illustrates a partial perspective view of an example housing of the handle assembly shown in FIG. 68;

FIG. 74 illustrates an example guide member for the handle assembly of FIG. 68;

FIG. 75 illustrates an example guide member for the handle assembly of FIG. 68;

FIG. 76 illustrates a transverse cross-section of the handle assembly of FIG. 68, where a follower of the handle assembly is shown in a first position relative to a housing of the handle assembly;

FIG. 77 illustrates a transverse cross-section of the handle assembly of FIG. 68, where the follower of FIG. 76 is shown in a second position relative to a housing of the handle assembly;

FIG. 78 illustrates the example follower of FIGS. 76 and 62 for the handle assembly of FIG. 68;

FIG. 79 illustrates a partial exploded view for the handle assembly of FIG. 68 showing an example guide member and follower of the handle assembly;

FIG. 80 illustrates a transverse cross-section of the handle assembly of FIG. 68, where a follower of the handle assembly is shown in a first position relative to a driver of the handle assembly;

FIG. 81 illustrates a transverse cross-section of the handle assembly of FIG. 68, where the follower of FIG. 80 is shown in a second position relative to the driver of the handle assembly;

FIG. 82 illustrates a partial exploded view of the handle assembly of FIG. 68 showing an engagement between a driver and a housing and guide member of the handle assembly;

FIG. 83 illustrates another partial exploded view of the engagement between the driver and housing and guide member of FIG. 82;

FIG. 84 illustrates a partial perspective view of the handle assembly of FIG. 68 showing an engagement between an actuation member and a housing of the handle assembly;

FIG. 85 illustrates another exploded view of the engagement between the actuation member and housing of FIG. 84;

FIG. 86 illustrates an example of a handle assembly having a housing that has a first shape;

FIG. 87 illustrates an example of a handle assembly having a housing that has a second shape;

FIG. 88 illustrates a cross section of the handle assembly of FIG. 86;

FIG. 89 illustrates a cross section of the handle assembly of FIG. 87;

FIG. 90 illustrates an example pull wire connector for a steerable catheter assembly;

FIG. 91 illustrates a cross section of the pull wire connector of FIG. 90;

FIG. 92 illustrates a cross-section view of an example pull wire connector for a steerable catheter assembly;

FIG. 93 illustrates a bottom view of the pull wire connector of FIG. 92;

FIG. 94 illustrates a cross-section view of an example pull wire connector for a steerable catheter assembly;

FIG. 95 illustrates a perspective view of an example pull wire connector for a steerable catheter assembly;

FIG. 96 illustrates a perspective view of an example pull wire connector for a steerable catheter assembly;

FIG. 97 illustrates the pull wire connector of FIG. 96 with wires attached thereto, where the wires are in a parallel configuration;

FIG. 98 illustrates the pull wire connector of FIG. 96 with wires attached thereto, where the wires are in a crossed configuration;

FIG. 99 illustrates a pull wire connector attached to a follower of a handle assembly for a steerable catheter assembly;

FIG. 100 illustrates an example pull wire connector attached to a follower of a handle assembly for a steerable catheter assembly;

FIG. 101 illustrates an example steerable catheter assembly being positioned in the heart of a patient, where an actuation member of a handle assembly of the steerable catheter assembly is rotated in a first direction;

FIG. 102 illustrates a partial cross-section of a handle assembly of the steerable catheter assembly of FIG. 101, where a pull wire connector of the handle assembly is in a first position relative to a follower of the handle assembly;

FIG. 103 illustrates the steerable catheter assembly of FIG. 101 being positioned in the heart of a patient, where an actuation member of a handle assembly of the steerable catheter assembly is rotated in a second direction;

FIG. 104 illustrates a partial cross-section of a handle assembly of the steerable catheter assembly of FIG. 103, where a pull wire connector of the handle assembly is in a first position relative to a follower of the handle assembly;

FIGS. 105-108 illustrate top, side, front and back views of an example of a connector having projections, posts, or cleats;

FIG. 109 illustrates the connector of FIGS. 105-108 mounted to follower device;

FIG. 110 illustrates a first winding pattern for a wire or actuation element having clockwise loops;

FIG. 111 illustrates a second winding pattern similar to the first winding pattern, but using more than one wire or actuation element;

FIG. 112 illustrates a third winding pattern for a wire or actuation element having counterclockwise loops;

FIG. 113 illustrates a fourth winding pattern similar to the first winding pattern, except that a counterclockwise loop is used for a middle post or cleat;

FIG. 114 illustrates a fifth winding pattern similar to that shown in FIG. 113, except the winding directions have been reversed from that shown in FIG. 113;

FIGS. 115 and 116 are similar to FIGS. 113 and 114, respectively, except that more than one wire or actuation element are used;

FIGS. 117 and 118 illustrate another winding pattern using a figure “8” looping arrangement around the posts or cleats;

FIG. 119 illustrates another winding pattern using a partial figure “8” loop around the post or cleats;

FIGS. 120 and 121 illustrates connector implementations having a secondary, elevated base and two or more posts or cleats;

FIG. 122 illustrates another implementation of a connector device having curved or flared head portions;

FIG. 123 illustrates another implementation of a connector device having diamond shaped head portions;

FIG. 124 illustrates another implementation of a connector device having one or more split projections, posts, or cleats;

FIG. 124A-124H illustrate additional implementations of connector devices;

FIG. 125 illustrates an example seal assembly for a handle assembly of a steerable catheter assembly, showing a seal housing and seal member of the seal assembly;

FIG. 126 illustrates a perspective view of an example cap for the seal assembly of FIG. 125;

FIG. 127 illustrates another perspective view of the cap of FIG. 126;

FIG. 128 illustrates an example guide member disposed within an example housing for a handle assembly of a steerable catheter assembly;

FIG. 129 illustrates the guide member of FIG. 129;

FIG. 130 illustrates a partial perspective view of a distal portion of the guide member of FIG. 128, where the distal portion has an example connection element;

FIG. 131 illustrates a partial perspective view of a proximal portion of the guide member of FIG. 128, where the proximal portion has an example connection element;

FIG. 132 illustrates a perspective view of an example catheter coupler for a steerable catheter assembly;

FIG. 133 illustrates an exploded view of various components of the catheter coupler of FIG. 132;

FIG. 134 illustrates an example catheter coupler housing of the catheter coupler of FIG. 132;

FIG. 135 illustrates a side view of the catheter coupler of FIG. 132;

FIG. 136 illustrates a cross section of the catheter coupler of FIG. 132 taken along the line 101-101 shown in FIG. 135, showing an example of an internal configuration of the catheter coupler;

FIG. 137 illustrates a cross section of the catheter coupler of FIG. 132 taken along the line 101-101 shown in FIG. 135, showing an example of an internal configuration of the catheter coupler; and

FIG. 138 illustrates a cross section of the catheter coupler of FIG. 132 taken along the line 101-101 shown in FIG. 135, showing an example of an internal configuration of the catheter coupler.

### DETAILED DESCRIPTION

The following description refers to the accompanying drawings, which illustrate some implementations of the present disclosure. Some implementations having different structures and operation do not depart from the scope of the present disclosure.

Some implementations of the present disclosure are directed to systems, devices, methods, etc. for repairing a defective heart valve. For example, some implementations of valve treatment devices, valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible.

Further, the techniques, methods, operations, steps, etc. described or suggested herein or in the references incorporated herein can be performed on a living subject (e.g., human, other animal, etc.) or on a simulation, such as a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of). The terms “clasp” and “clasp arm” are often used herein with respect to specific examples, but the terms “gripping member” and/or “gripper arm” can be used in place of and function in the same or similar ways, even if not configured in the same way as a typical clasp.

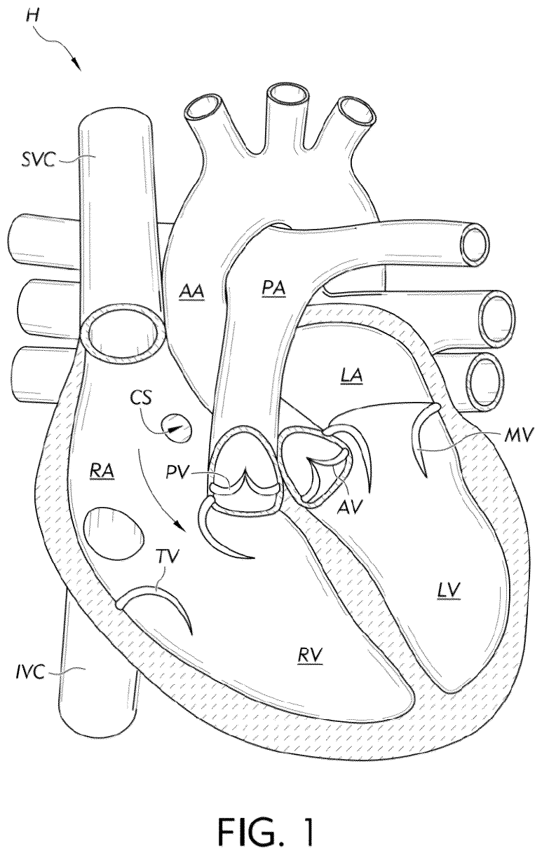

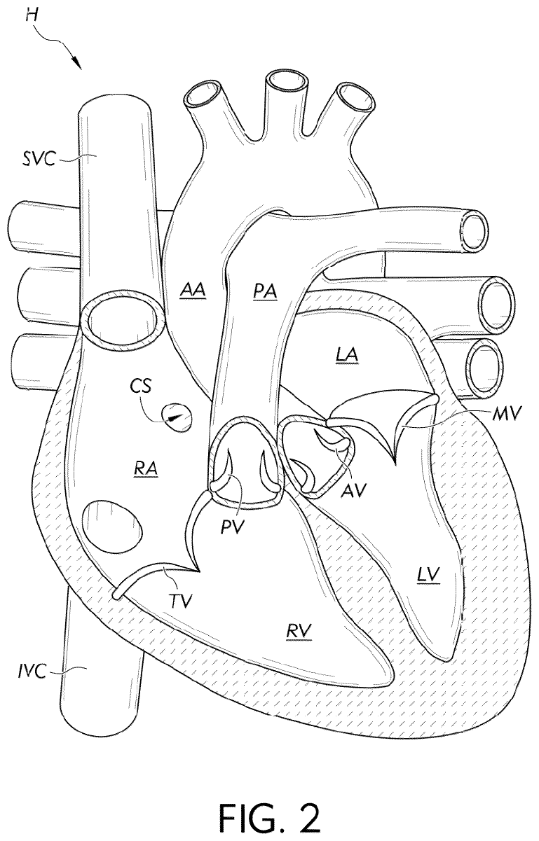

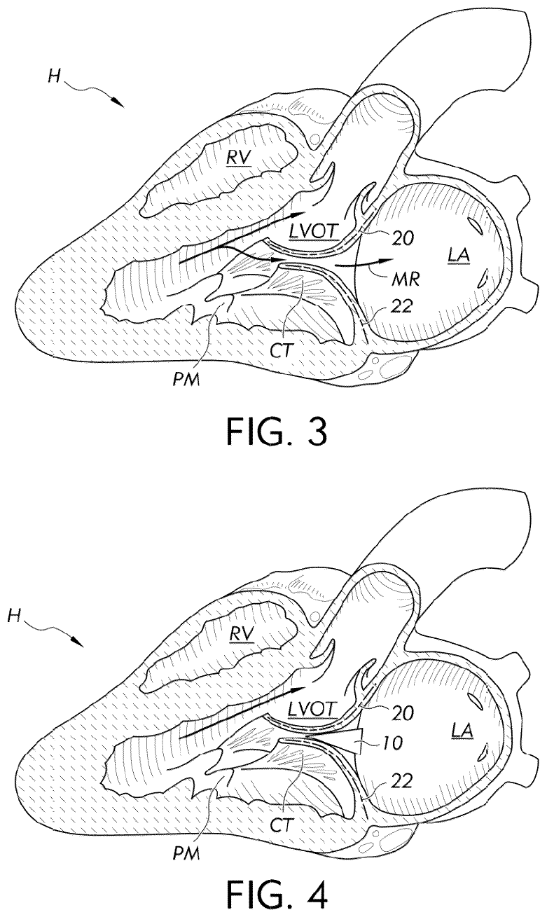

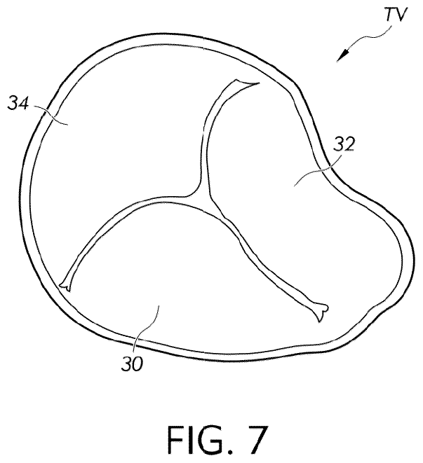

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair and/or treatment systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit or reduce blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

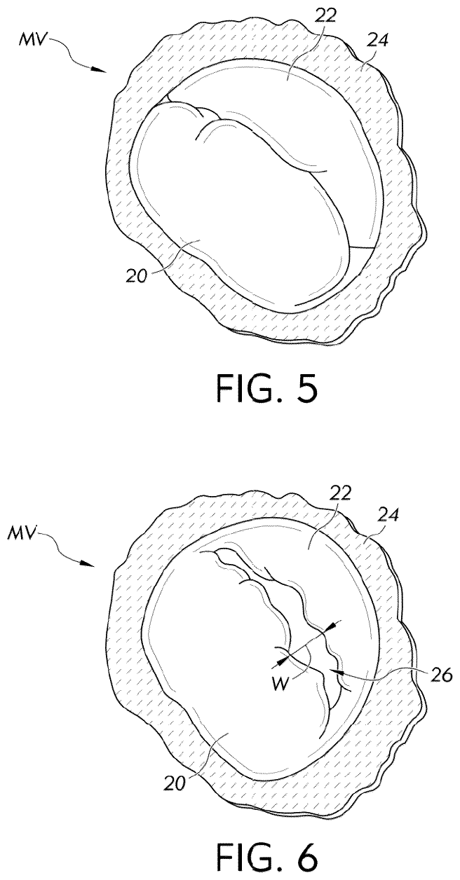

Referring now to FIGS. 1-7, the mitral valve MV includes two leaflets, the anterior leaflet 20 and the posterior leaflet 22. The mitral valve MV also includes an annulus 24 (see FIG. 5), which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 may have a width W between about 2.5 mm and about 17.5 mm, between about 5 mm and about 15 mm, between about 7.5 mm and about 12.5 mm, or about 10 mm. In some situations, the gap 26 may have a width W greater than 15 mm or even 17.5 mm. As set forth above, there are several different ways that a leaflet (e.g., leaflets 20, 22 of mitral valve MV) may malfunction which may thereby lead to valvular regurgitation.

In any of the above-mentioned situations, a device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of a repair or treatment device 10 (e.g., a valve treatment device, a valve repair device, an implantable device, an implant, etc.) is shown implanted between the leaflets 20, 22 such that regurgitation does not occur during systole (compare FIG. 3 with FIG. 4). In some implementations, the coaptation element (e.g., spacer, coaption element, gap filler, membrane, sheet, plug, wedge, balloon, etc.) of the device 10 has a generally tapered or triangular shape that naturally adapts to the native valve geometry and to its expanding leaflet nature (toward the annulus). In this application, the terms spacer, coaption element, coaptation element, gap filler, plug, etc. are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another).

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the devices or implants provided herein can be centrally located between the three leaflets 30, 32, 34.

An example device or implant can optionally have a coaptation element (e.g., spacer, coaption element, gap filler, membrane, sheet, plug, wedge, balloon, etc.) and at least one anchor (e.g., one, two, three, or more). In some implementations, a device (e.g., treatment device, repair device, implantable device, implant, etc.) can have any combination or sub-combination of the features disclosed herein without a coaptation element. When included, the coaptation element (e.g., spacer, coaption element, gap filler, membrane, sheet, plug, wedge, balloon, etc.) is configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaptation element, gap filler, membrane, sheet, plug, wedge, balloon, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surfaces extend between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element (e.g., an actuation shaft, actuation tube, actuation wire, etc.) to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when deployed such that the leaflet is grasped by the anchor.

The device or implant can be configured to be deployed via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation and/or deployment methods for some implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

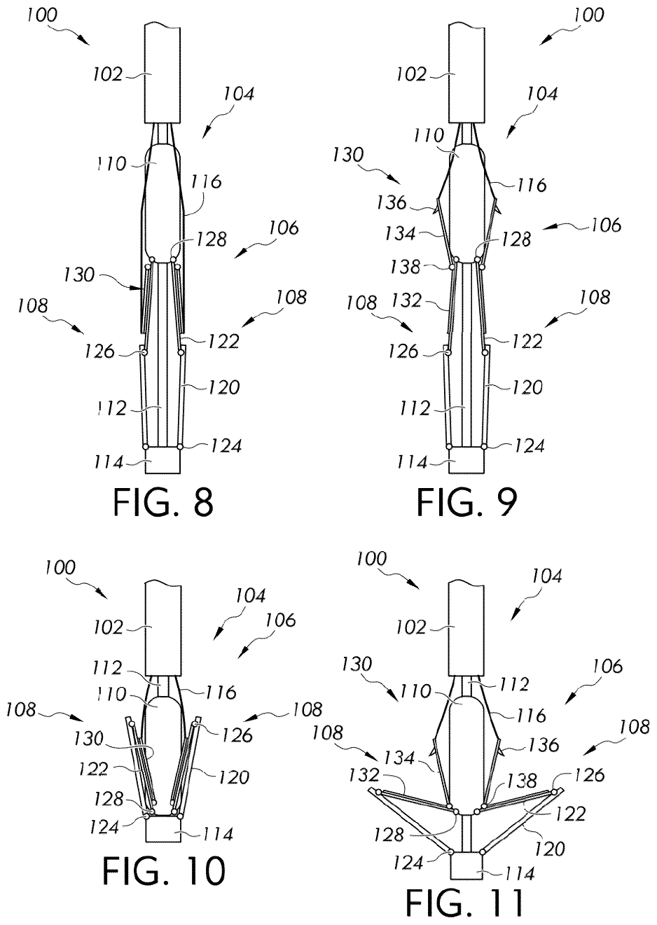

Referring now to FIGS. 8-15, a schematically illustrated device or implant 100 (e.g., a prosthetic device, a valve repair device, implantable device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety. The device 100 can include any other features for another device or implant discussed in the present application or the applications cited above, and the device 100 can be positioned to engage valve tissue (e.g., leaflets 20, 22, 30, 32, 34) as part of any suitable treatment and/or repair system (e.g., any valve repair system and/or valve treatment system disclosed in the present application or the applications cited above).

The device or implant 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion 104 and an anchor portion 106.

In some implementations, the coaptation portion 104 of the device or implant 100 includes a coaptation element 110 that is adapted to be implanted between leaflets of a native valve (e.g., a native mitral valve, native tricuspid valve, etc.) and is slidably attached to an actuation element 112 (e.g., actuation wire, shaft, tube, hypotube, line, suture, braid, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during deployment and/or implantation. The actuation element 112 (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and a coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and the actuation element 112 (e.g., actuation wire, shaft, tube, hypotube, line, suture, braid, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element (e.g., clamp, clip, lock, sutures, friction fit, buckle, snap fit, lasso, etc.) removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members (e.g., gripping arms, clasp arms, etc.). The illustrated gripping members can comprise clasps 130 that include a base or fixed arm 132, a moveable arm 134, optional friction-enhancing elements, other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate the coaptation element 110. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the optional barbs or other friction-enhancing elements 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter, an implant catheter, etc.). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs or other friction-enhancing elements 136 of the clasps 130 can grab, pinch, and/or pierce the native leaflets to further secure the native leaflets.

During deployment and/or implantation, the paddles 120, 122 can be opened and closed, for example, to grasp the native leaflets (e.g., native mitral valve leaflets, etc.) between the paddles 120, 122 and/or between the paddles 120, 122 and a coaptation element 110 (e.g., a spacer, plug, membrane, etc.). The clasps 130 can be used to grasp and/or further secure the native leaflets by engaging the leaflets with optional barbs or other friction-enhancing elements 136 and pinching the leaflets between the moveable and fixed arms 134, 132. The optional barbs or other friction-enhancing elements 136 (e.g., protrusions, ridges, grooves, textured surfaces, adhesive, etc.) of the clasps 130 increase friction with the leaflets or can partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from a delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system. The actuation lines 116 can extend and attach to the moveable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and moveable arms 132, 134 of the clasps 130.

Referring now to FIG. 10, the device 100 is shown in a shortened or fully closed condition. To move the device 100 from the elongated condition to the shortened condition, the actuation element 112 is retracted to pull the cap 114 towards the coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11-13, the device 100 is shown in a partially open, grasp-ready condition. To transition from the fully closed to the partially open condition, the actuation element (e.g., actuation wire, shaft, tube, hypotube, line, suture, braid, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., actuation wire, shaft, tube, hypotube, line, suture, braid, etc.) and cap (or other attachment portion) are used to control one paddle, and the other independent actuation element and cap (or other attachment portion) are used to control the other paddle.

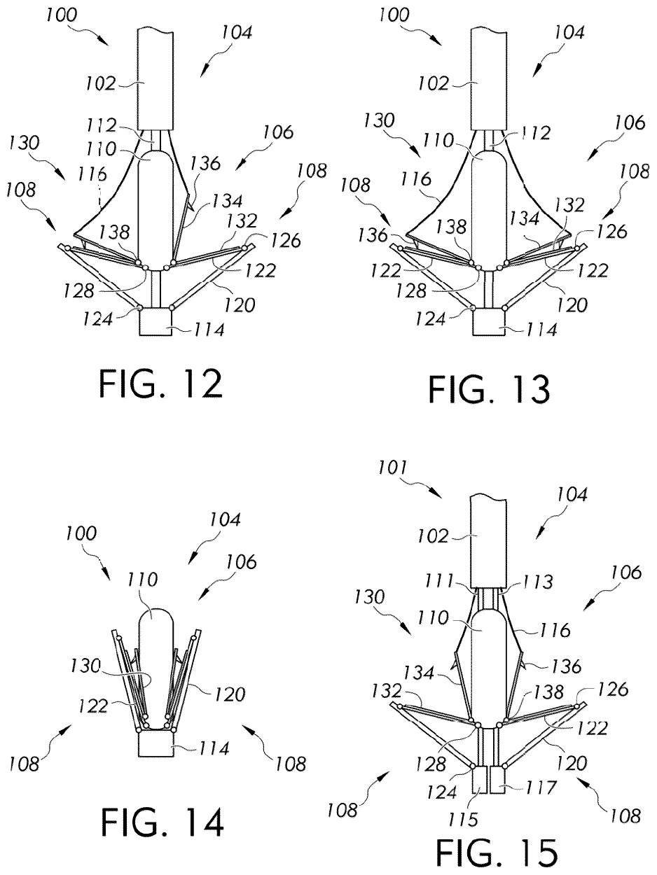

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the coaptation element 110 and the clasps 130 pinched around native leaflets. Similarly, the fixed and moveable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after deployment and/or implantation.

FIG. 15 illustrates an example where the paddles 120, 122 are independently controllable. The device 101 illustrated by FIG. 15 is similar to the device illustrated by FIG. 11, except the device 100 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the spacer or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

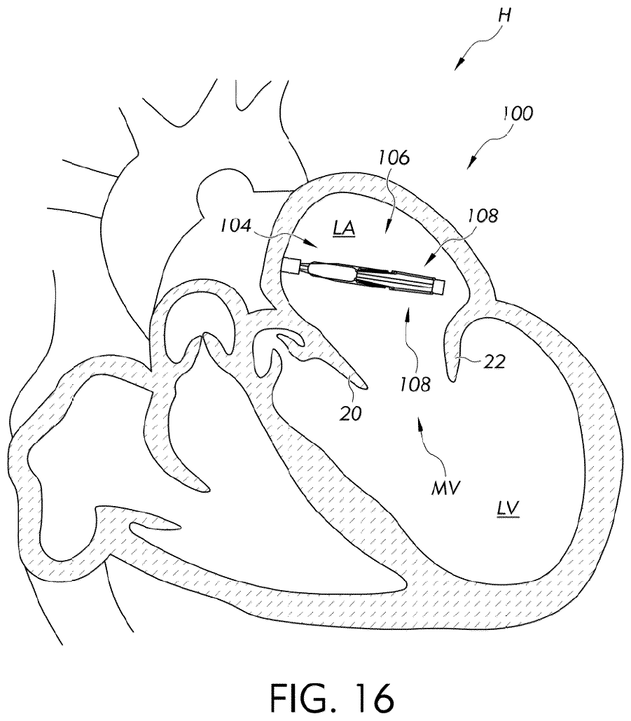

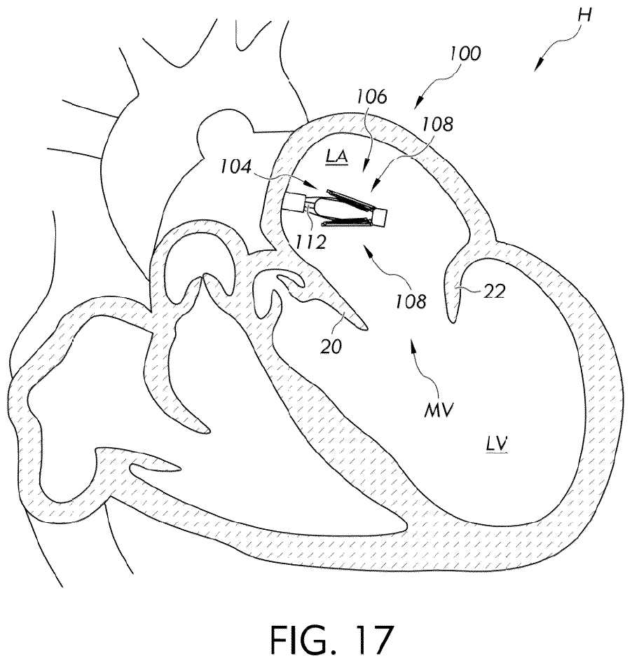

Referring now to FIGS. 16-21, the device 100 of FIGS. 8-14 is shown being delivered and deployed within the native mitral valve MV of the heart H. Referring to FIG. 16, a delivery sheath/catheter is inserted into the left atrium LA through the septum and the device/implant 100 is deployed from the delivery catheter/sheath in the fully open condition as illustrated in FIG. 16. The actuation element 112 is then retracted to move the device/implant into the fully closed condition shown in FIG. 17.

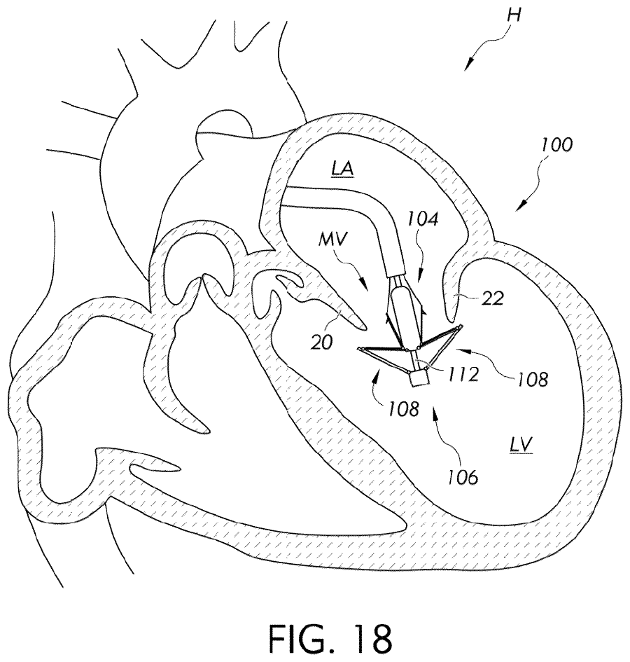

As can be seen in FIG. 18, the device/implant is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The device or implant catheter connected to the device/implant can be advanced from inside the steerable catheter to position the device/implant as illustrated by FIG. 18.

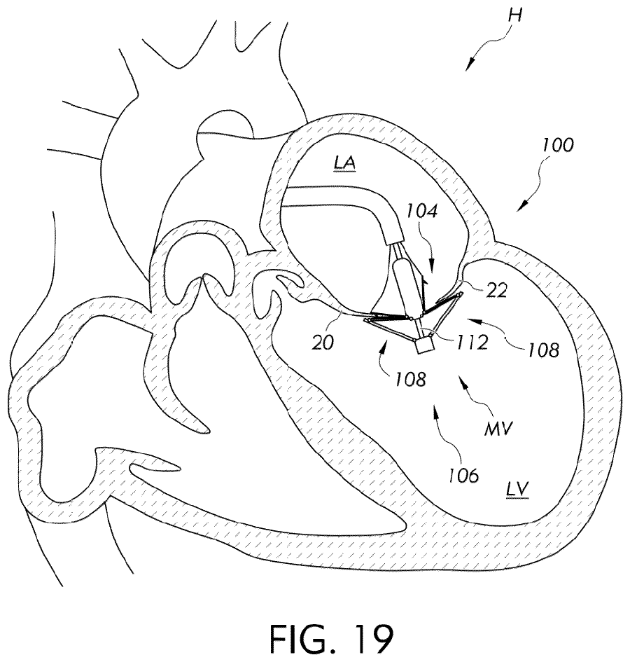

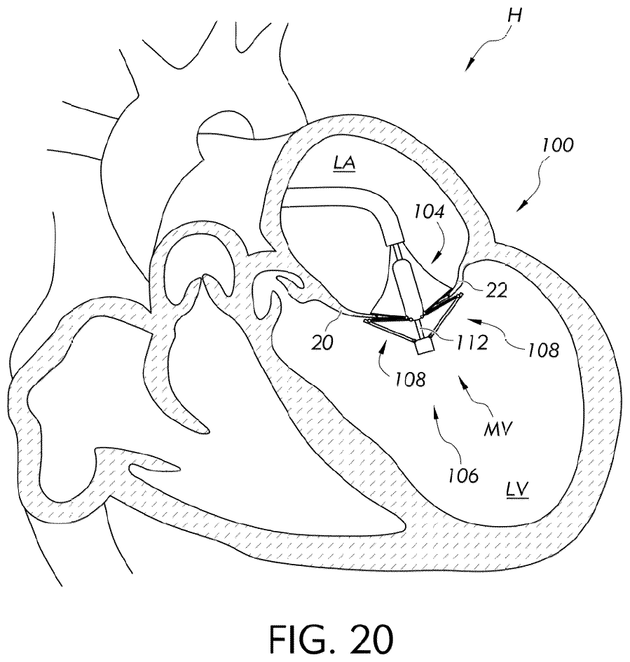

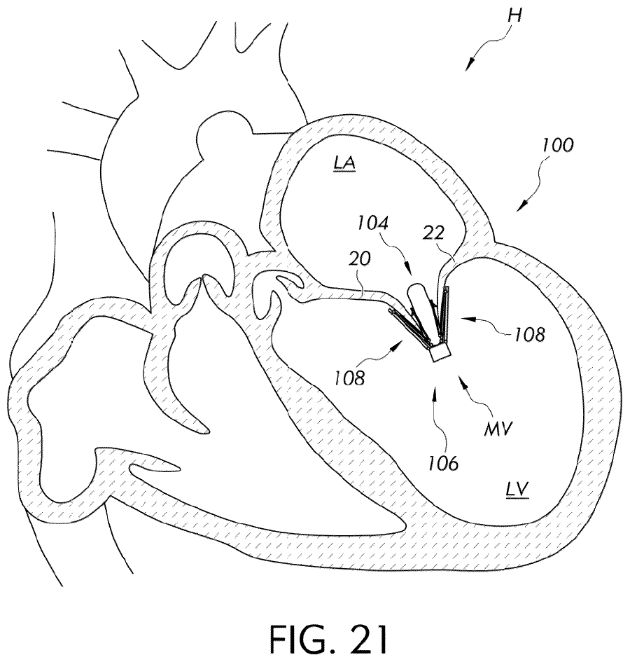

Referring now to FIG. 19, the device catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device or implant 100 is fully closed and deployed in the native mitral valve MV.

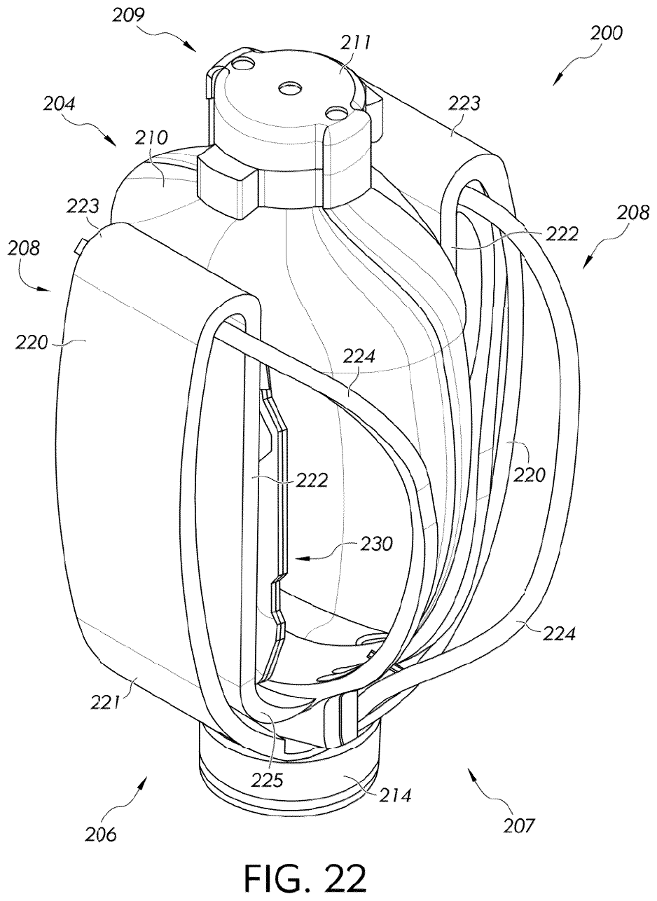

Any of the features disclosed by the present application can be used in a wide variety of different treatment and/or repair devices. FIGS. 22-24 illustrate examples of treatment and/or repair devices that can be modified to include any of the features disclosed by the present application. Any combination or sub-combination of the features disclosed by the present application can be combined with, substituted for, and/or added to any combination or sub-combination of the features of the devices illustrated by FIGS. 8-24.

Referring now to FIG. 22, an example of a device 200 (e.g., treatment device, repair device, implantable device, implant, etc.) is shown. The device 200 is one of the many different configurations that the device 100 that is schematically illustrated in FIGS. 8-14 can take. The device 200 can include any other features for a device or implant discussed in the present application, and the device 200 can be positioned to engage valve tissue 20, 22 as part of any suitable treatment and/or repair system (e.g., any valve repair system and/or treatment system disclosed in the present application). The device/implant 200 can be a prosthetic spacer device, valve repair device, treatment device, or another type of device that attaches to leaflets of a native valve.

In some implementations, the device or implant 200 includes a coaptation portion 204, a proximal or attachment portion 209, an anchor portion 206, and a distal portion 207. In some implementations, the coaptation portion 204 of the device optionally includes a coaptation element 210 (e.g., a spacer, coaption element, plug, membrane, sheet, gap filler, plug, wedge, balloon, etc.) for deployment and/or implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 209 includes a first or proximal collar 211 (or other attachment element) for engaging with a capture mechanism of a delivery system. A delivery system for the device 200 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc. The capture mechanism can be configured in a variety of ways and, in some implementations, can comprise one or more of a clamp, clip, pin, suture, line, lasso, noose, snare, buckle, lock, latch, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for deployment and/or implantation in the human body.

An actuation element (e.g., actuation wire, shaft, tube, hypotube, line, suture, braid, etc.) can extend from a delivery system (not shown) to engage and enable actuation of the device or implant 200. In some implementations, the actuation element extends through the proximal collar 211, and spacer or coaptation element 210 to engage a cap 214 of the distal portion 207. The actuation element can be configured to removably engage the cap 214 with a threaded connection, or the like, so that the actuation element can be disengaged and removed from the device 200 after implantation.

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above and has a tapered shape or cross-section when seen from a front view and a round shape or cross-section when seen from a side view. A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed portion of the clasps 230. The inner paddle 222, the outer paddle 220, and the coaptation element can all be interconnected as described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

The wide configuration of the paddle frames 224 provides increased surface area compared to the inner paddles 222 alone. The increased surface area can distribute the clamping force of the paddles 220 and paddle frames 224 against the native leaflets over a relatively larger surface of the native leaflets in order to further protect the native leaflet tissue.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215). Any combination or sub-combination of the features disclosed by the present application can be combined with any combination or sub-combination of the features disclosed by Patent Cooperation Treaty International Application No. PCT/US2018/028189 (International Publication No. WO 2018/195215). Patent Cooperation Treaty International Application No. PCT/US2018/028189 (International Publication No. WO 2018/195215) is incorporated herein by reference in its entirety.

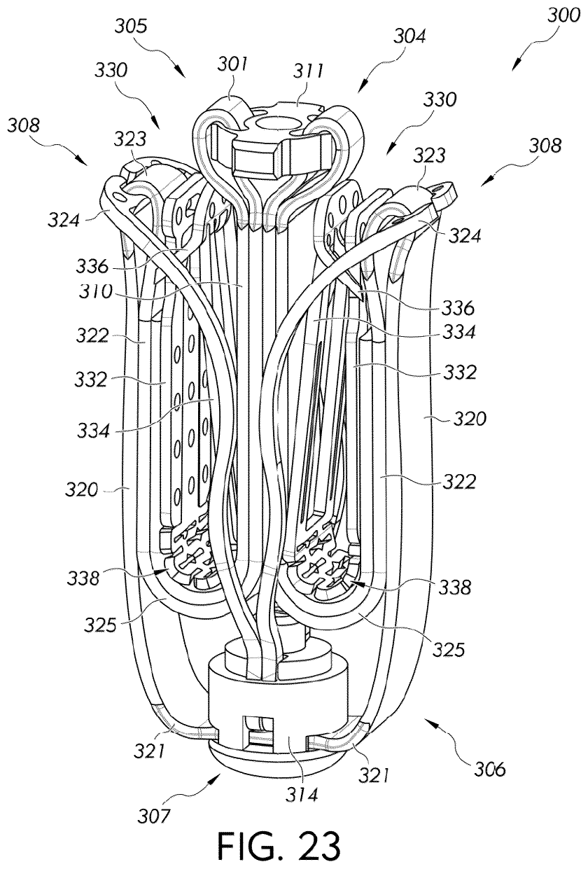

Referring now to FIG. 23, an example of a device or implant 300 is shown. The device 300 is one of the many different configurations that the device 100 that is schematically illustrated in FIGS. 8-14 can take. The device 300 can include any other features for a device or implant discussed in the present application, and the device 300 can be positioned to engage valve tissue 20, 22 as part of any suitable treatment and/or repair system (e.g., any valve repair system and/or treatment system disclosed in the present application).

The device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for deployment and/or implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism of a delivery system.

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In implementations with a coaptation element 310, the coaptation element 310 and the anchors 308 can be coupled together in various ways. As shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip 301.

Like the anchors 208 of the device or implant 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the spacer or coaptation element 310. From the straight configuration, the anchors 308 can be moved to a fully folded configuration (e.g., FIG. 23), e.g., by moving the proximal end and distal end toward each other and/or toward a midpoint or center of the device.

In some implementations, the clasps comprise a moveable arm coupled to an anchor. In some implementations, the clasps 330 include a base or fixed arm 332, a moveable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

The fixed arms 332 are attached to the inner paddles 322 through holes or slots with sutures. The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose the optional barbs 336. The clasps 330 are opened by applying tension to actuation lines attached to the moveable arms 334, thereby causing the moveable arms 334 to articulate, pivot, and/or flex on the joint portions 338.

In short, the device or implant 300 is similar in configuration and operation to the device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 23 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 23, the strip of material 301 begins and ends in the location of the inner paddles 322.

As with the device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898). Any combination or sub-combination of the features disclosed by the present application can be combined with any combination or sub-combination of the features disclosed by Patent Cooperation Treaty International Application No. PCT/US2019/055320 (International Publication No. WO 2020/076898). Patent Cooperation Treaty International Application No. PCT/US2019/055320 (International Publication No. WO 2020/076898) is incorporated herein by reference in its entirety.

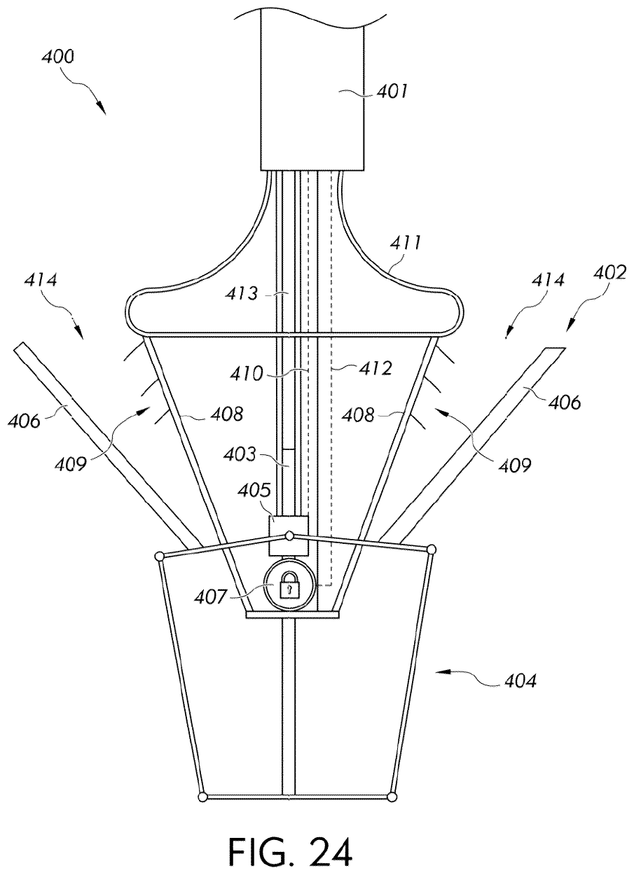

FIG. 24 illustrates another example of one of the many treatment and/or repair systems 400 useable for repairing a native valve of a patient that the concepts of the present application can be applied to. The system 400 includes a delivery device 401 and a repair and/or treatment device 402.

The treatment and/or repair device 402 includes a base assembly 404, a pair of paddles 406, and a pair of gripping members 408 (e.g., clasps, clasp arms, grippers, gripping arms, latches, etc.). In one example, the paddles 406 can be integrally formed with the base assembly. For example, the paddles 406 can be formed as extensions of links of the base assembly. In the illustrated example, the base assembly 404 of the device 402 has a shaft 403, a coupler 405 configured to move along the shaft, and a lock 407 configured to lock the coupler in a stationary position on the shaft. The coupler 405 is mechanically connected to the paddles 406, such that movement of the coupler 405 along the shaft 403 causes the paddles to move between an open position and a closed position. In this way, the coupler 405 serves as a means for mechanically coupling the paddles 406 to the shaft 403 and, when moving along the shaft 403, for causing the paddles 406 to move between their open and closed positions.

In some implementations, the gripping members 408 are pivotally connected to the base assembly 404 (e.g., the gripping members 408 can be pivotally connected to the shaft 403, or any other suitable member of the base assembly), such that the gripping members can be moved to adjust the width of the opening 414 between the paddles 406 and the gripping members 408. The gripping member 408 can include a barbed portion 409 for attaching the gripping members to valve tissue when the device 402 is attached to the valve tissue. When the paddles 406 are in the closed position, the paddles engage the gripping members 408, such that, when valve tissue is attached to the barbed portion 409 of the gripping members, the paddles secure the device 402 to the valve tissue. In some implementations, the gripping members 408 are configured to engage the paddles 406 such that the barbed portion 409 engages the valve tissue member and the paddles 406 to secure the device 402 to the valve tissue member. For example, in certain situations, it can be advantageous to have the paddles 406 maintain an open position and have the gripping members 408 move outward toward the paddles 406 to engage valve tissue and the paddles 406.

While the example shown in FIG. 24 illustrates a pair of paddles 406 and a pair of gripping members 408, it should be understood that the device 402 can include any suitable number of paddles and gripping members.

In some implementations, the system 400 includes a placement shaft 413 that is removably attached to the shaft 403 of the base assembly 404 of the treatment and/or repair device 402. In some implementations, after the treatment and/or repair device 402 is secured to valve tissue, the placement shaft 413 can be removed from the shaft 403 to remove the treatment and/or repair device 402 from the remainder of the system 400, such that the treatment and/or repair device 402 can remain attached to the valve tissue, and the delivery device 401 can be removed from a patient's body.

The treatment and/or repair system 400 can also include a paddle control mechanism 410, a gripper control mechanism 411, and a lock control mechanism 412. The paddle control mechanism 410 is mechanically attached to the coupler 405 to move the coupler along the shaft, which causes the paddles 406 to move between the open and closed positions. The paddle control mechanism 410 can take any suitable form, and can comprise, for example, a shaft, wire, tube, hypotube, rod, suture, line, etc. For example, the paddle control mechanism can comprise a hollow shaft, a catheter tube or a sleeve that fits over the placement shaft 413 and the shaft 403 and is connected to the coupler 405.

The gripper control mechanism 411 is configured to move the gripping members 408 such that the width of the opening 414 between the gripping members and the paddles 406 can be altered. The gripper control mechanism 411 can take any suitable form, such as, for example, a line, a suture or wire, a rod, a catheter, a tube, a hypotube, etc.

The lock control mechanism 412 is configured to lock and unlock the lock. The lock 407 locks the coupler 405 in a stationary position with respect to the shaft 403 and can take a wide variety of different forms and the type of lock control mechanism 412 can be dictated by the type of lock used. In examples in which the lock 407 includes a pivotable plate, the lock control mechanism 412 is configured to engage the pivotable plate to move the plate between the tilted and substantially non-tilted positions. The lock control mechanism 412 can be, for example, a rod, a suture, a wire, or any other member that is capable of moving a pivotable plate of the lock 407 between a tilted and substantially non-tilted position.

The device 402 is movable from an open position to a closed position. The base assembly 404 includes links that are moved by the coupler 405. The coupler 405 is movably attached to the shaft 403. In order to move the device from the open position to the closed position, the coupler 405 is moved along the shaft 403, which moves the links.

The gripper control mechanism 411 moves the gripping members 408 to provide a wider or a narrower gap at the opening 414 between the gripping members and the paddles 406. In the illustrated example, the gripper control mechanism 411 includes a line, such as a suture, a wire, etc. that is connected to an opening in an end of the gripping members 408. When the line(s) is pulled, the gripping members 408 move inward, which causes the opening 414 between the gripping members and the paddles 406 to become wider.

In order to move the device 402 from the open position to the closed position, the lock 407 is moved to an unlocked condition by the lock control mechanism 412. Once the lock 407 is in the unlocked condition, the coupler 405 can be moved along the shaft 403 by the paddle control mechanism 410.

After the paddles 406 are moved to the closed position, the lock 407 is moved to the locked condition by the lock control mechanism 412 to maintain the device 402 in the closed position. After the device 402 is maintained in the locked condition by the lock 407, the device 402 is removed from the delivery device 401 by disconnecting the shaft 403 from the placement shaft 413. In addition, the device 402 is disengaged from the paddle control mechanism 410, the gripper control mechanism 411, and the lock control mechanism 412.

Additional features of the device 402, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904). Patent Cooperation Treaty International Application No. PCT/US2019/012707 (International Publication No. WO 2019139904) is incorporated herein by reference in its entirety.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

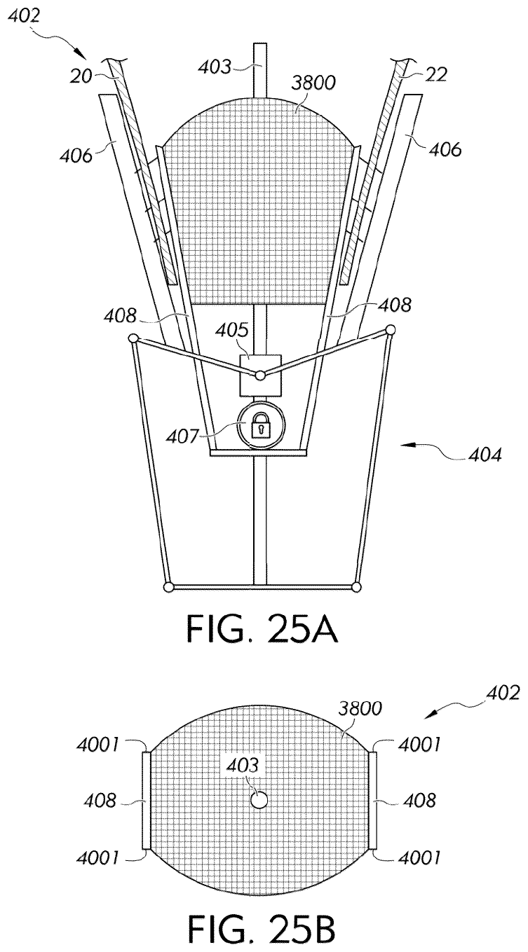

Referring to FIGS. 25A-25B, an example implementation of a treatment and/or repair device 402 has a coaptation element 3800. The device 402 can have the same configuration as the device illustrated by FIG. 24 with the addition of the coaptation element. The coaptation element 3800 can take a wide variety of different forms. The coaptation element 3800 can be compressible and/or expandable. For example, the coaptation element can be compressed to fit inside one or more catheters of a delivery system, can expand when moved out of the one or more catheters, and/or can be compressed by the paddles 406 to adjust the size of the coaptation element. In the example illustrated by FIGS. 25A and 25B, the size of the coaptation element 3800 can be reduced by squeezing the coaptation element with the paddles 406 and can be increased by moving the paddles 406 away from one another. The coaptation element 3800 can extend past outer edges 4001 of the gripping members or clasps 408 as illustrated for providing additional surface area for closing the gap of a mitral valve.

The coaptation element 3800 can be coupled to the device 402 in a variety of different ways. For example, the coaptation element 3800 can be fixed to the shaft 403, can be slidably disposed around the shaft, can be connected to the coupler 405, can be connected to the lock 407, and/or can be connected to a central portion of the clasps or gripping members 408. In some implementations, the coupler 405 can take the form of the coaptation element 3800. That is, a single element can be used as the coupler 405 that causes the paddles 406 to move between the open and closed positions and the coaptation element 3800 that closes the gap between the leaflets 20, 22 when the device 402 is attached to the leaflets.

The coaptation element 3800 can be disposed around one or more of the shafts or other control elements of the system 400. For example, the coaptation element 3800 can be disposed around the shaft 403, the shaft 413, the paddle control mechanism 410, and/or the lock control mechanism 412.

The device 402 can include any other features for a treatment and/or repair device discussed in the present application, and the device 402 can be positioned to engage valve tissue as part of any suitable treatment and/or repair system (e.g., any valve repair system and/or treatment systems disclosed in the present application). Additional features of the device 402, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904).

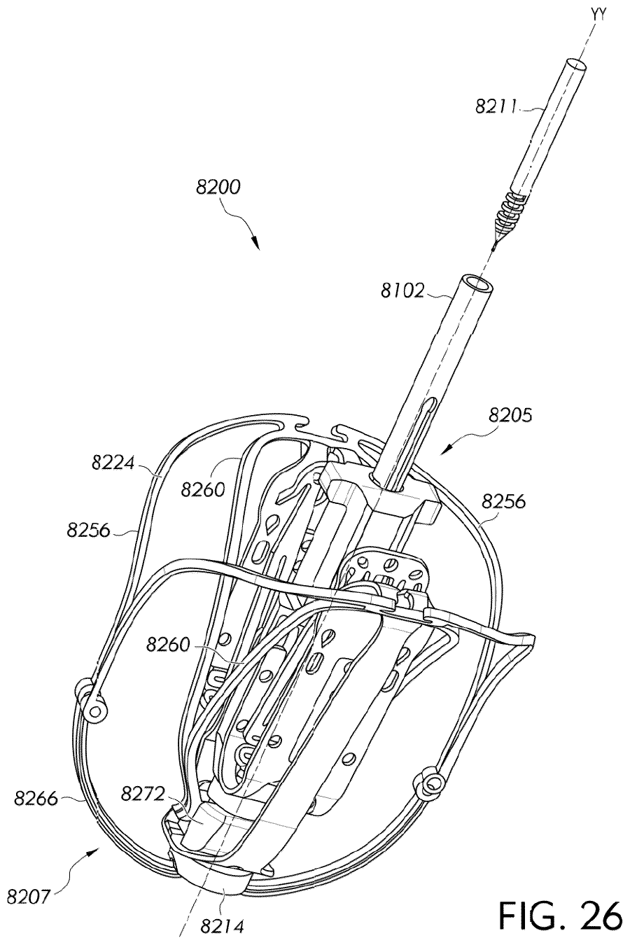

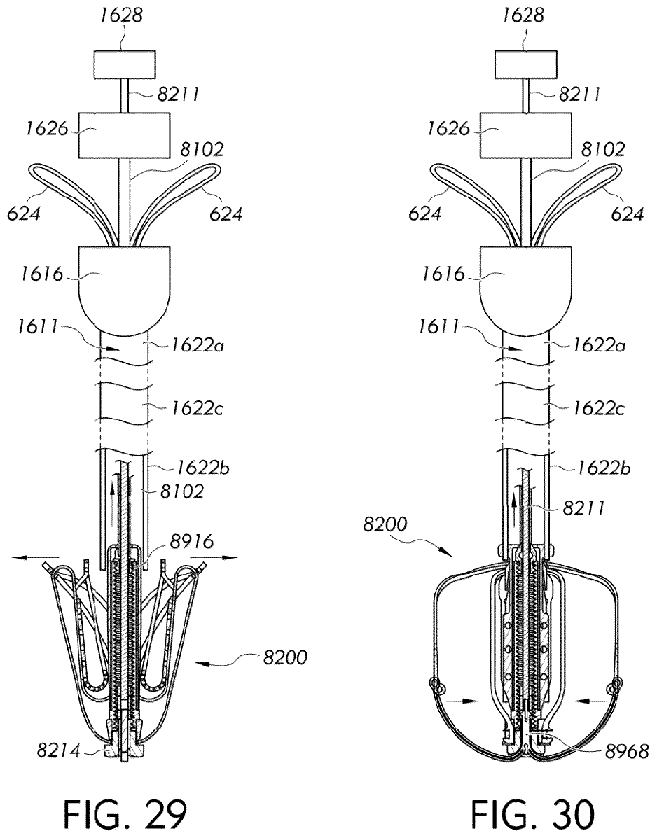

FIGS. 26-30 illustrate an example of one of the many systems for treating and/or repairing a native valve of a patient that the concepts of the present application can be applied to. Referring to FIGS. 29 and 30, the system includes a catheter assembly 1611 (e.g., a device catheter assembly, an implant catheter assembly, treatment catheter assembly, etc.) and a repair or treatment device 8200. Referring to FIGS. 26-28, the device 8200 includes a proximal or attachment portion 8205, paddle frames 8224, and a distal portion 8207. The attachment portion 8205, the distal portion 8207, and the paddle frames 8224 can be configured in a variety of ways.

In the example illustrated in FIG. 26, the paddle frames 8224 can be symmetric along longitudinal axis YY. However, in some implementations, the paddle frames 8224 are not symmetric about the axis YY. Moreover, referring to FIG. 26, the paddle frames 8224 include outer frame portions 8256 and inner frame portions 8260.

In some implementations, the connector 8266 (e.g., shaped metal component, shaped plastic component, tether, wire, strut, line, cord, suture, etc.) attaches to the outer frame portions 8256 at outer ends of the connector 8266 and to a coupler 8972 at an inner end 8968 of the connector 8266 (see FIG. 28). Between the connector 8266 and the attachment portion 8205, the outer frame portions 8256 form a curved shape. For example, in the illustrated example, the shape of the outer frame portions 8256 resembles an apple shape in which the outer frame portions 8256 are wider toward the attachment portion 8205 and narrower toward the distal portion 8207. In some implementations, however, the outer frame portions 8256 can be otherwise shaped.

The inner frame portions 8260 extend from the attachment portion 8205 toward the distal portion 8207. The inner frame portions 8260 then extend inward to form retaining portions 8272 that are attached to the actuation cap 8214. The retaining portions 8272 and the actuation cap 8214 can be configured to attach in any suitable manner.

In some implementations, the inner frame portions 8260 are rigid frame portions, while the outer frame portions 8256 are flexible frame portions. The proximal end of the outer frame portions 8256 connect to the proximal end of the inner frame portions 8260, as illustrated in FIG. 26.

The width adjustment element 8211 (e.g., width adjustment wire, width adjustment shaft, width adjustment tube, width adjustment line, width adjustment cord, width adjustment suture, width adjustment screw or bolt, etc.) is configured to move the outer frame portions 8256 from the expanded position to the narrowed position by pulling the inner end 8968 (FIG. 28) and portions of the connector 8266 into the actuation cap 8214. The actuation element 8102 is configured to move the inner frame portions 8260 to open and close the paddles in accordance with some implementations disclosed herein.

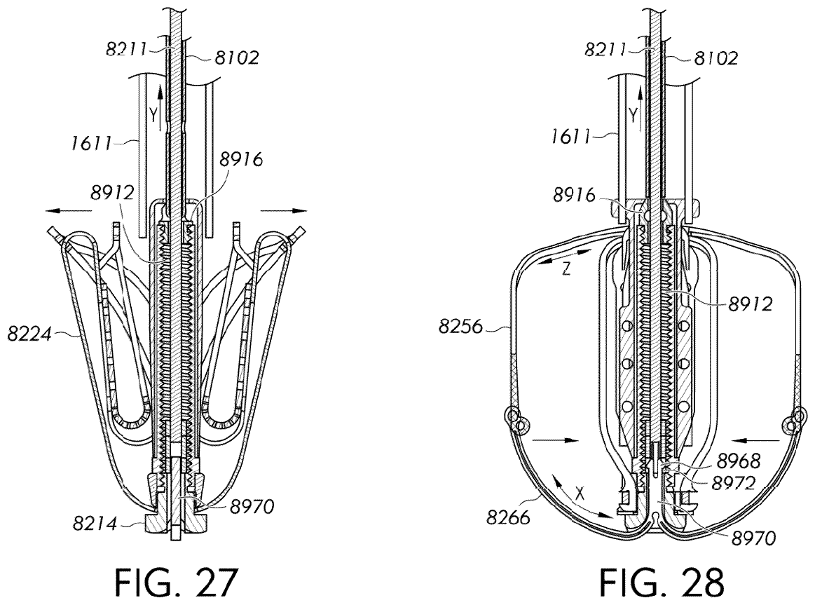

As shown in FIGS. 27 and 28, the connector 8266 has an inner end 8968 that engages with the width adjustment element 8211 such that a user can move the inner end 8968 inside the receiver 8912 (e.g., an internally threaded element, a column, a conduit, a hollow member, a notched receiving portion, a tube, a shaft, a sleeve, a post, a housing, a cylinder, tracks, etc.) to move the outer frame portions 8256 between a narrowed position and an expanded position. In the illustrated example, the inner end 8968 includes a post 8970 that attaches to the outer frame portions 8256 and a coupler 8972 that extends from the post 8970. The coupler 8972 is configured to attach and detach from both the width adjustment element 8211 and the receiver 8912. The coupler 8972 can take a wide variety of different forms. For example, the coupler 8972 can include one or more of a threaded connection, features that mate with threads, detent connections, such as outwardly biased arms, walls or other portions. When the coupler 8972 is attached to the width adjustment element 8211, the coupler is released from the receiver 8912. When the coupler 8972 is detached from the width adjustment element 8211, the coupler is secured to the receiver. The inner end 8968 of the connector can, however, be configured in a variety of ways. Any configuration that can suitably attach the outer frame portions 8256 to the coupler to allow the width adjustment element 8211 to move the outer frame portions 8256 between the narrowed position and the expanded position can be used. The coupler can be configured in a variety of ways as well and can be a separate component or be integral with another portion of the device, e.g., of the connector or inner end of the connector.

The width adjustment element 8211 allows a user to expand or contract the outer frame portions 8256 of the device 8200. In the example illustrated in FIGS. 27 and 28, the width adjustment element 8211 includes an externally threaded end that is threaded into the coupler 8972. The width adjustment element 8211 moves the coupler in the receiver 8912 to adjust the width of the outer frame portions 8256. When the width adjustment element 8211 is unscrewed from the coupler 8972, the coupler engages the inner surface of the receiver 8912 to set the width of the outer frame portions 8256.

In some implementations, the receiver 8912 can be integrally formed with a distal cap 8214. Moving the cap 8214 relative to a body of the attachment portion 8205 opens and closes the paddles. In the illustrated example, the receiver 8912 slides inside the body of the attachment portion. When the coupler 8972 is detached from the width adjustment element 8211, the width of the outer frame portions 8256 is fixed while the actuation element 8102 moves the receiver 8912 and cap 8214 relative to a body of the attachment portion 8205. Movement of the cap can open and close the device in the same manner as some implementations disclosed above.

In the illustrated example, a driver head 8916 is disposed at a proximal end of the actuation element 8102. The driver head 8916 releasably couples the actuation element 8102 to the receiver 8912. In the illustrated example, the width adjustment element 8211 extends through the actuation element 8102. The actuation element is axially advanced in the direction opposite to direction Y to move the distal cap 8214. Movement of the distal cap 8214 relative to the attachment portion 8205 is effective to open and close the paddles, as indicated by the arrows in FIG. 27. That is, movement of the distal cap 8214 in the direction Y closes the device and movement of the distal cap in the direction opposite to direction Y opens the device.

Also illustrated in FIGS. 27 and 28, the width adjustment element 8211 extends through the actuation element 8102, the driver head 8916, and the receiver 8912 to engage the coupler 8972 attached to the inner end 8968. The movement of the outer frame portions 8256 to the narrowed position can allow the device or implant 8200 to maneuver more easily into position for deployment and/or implantation in the heart by reducing the contact and/or friction between the native structures of the heart—e.g., chordae—and the device 8200. The movement of the outer frame portions 8256 to the expanded position provides the anchor portion of the device or implant 8200 with a larger surface area to engage and capture leaflet(s) of a native heart valve.

Referring to FIGS. 29 and 30, an example of a catheter assembly 1611 (e.g., a device catheter assembly, an implant catheter assembly, treatment catheter assembly, etc.) in which clasp actuation lines 624 extend through a handle 1616, the actuation element 8102 is coupled to a paddle actuation control 1626, and the width adjustment element 8211 is coupled to a paddle width control 1628. A proximal end portion 1622a of the shaft or catheter of the catheter assembly 1611 can be coupled to the handle 1616, and a distal end portion 1622b of the shaft or catheter can be coupled to the device 8200. The actuation element 8102 can extend distally from the paddle actuation control 1626, through the handle 1616, through the delivery shaft or catheter of the catheter assembly 1611, and through the proximal end of the device 8200, where it couples with the driver head 8916. The actuation element 8102 can be axially movable relative to the outer shaft of the catheter assembly 1611 and the handle 1616 to open and close the device.

The width adjustment element 8211 can extend distally from the paddle width control 1628, through the paddle actuation control 1626 and through the actuation element 8102 (and, consequently, through the handle 1616, the outer shaft of the catheter assembly 1611, and through the device 8200), where it couples with the movable coupler 8972. The width adjustment element 8211 can be axially movable relative to the actuation element 8102, the outer shaft of the catheter assembly 1611, and the handle 1616. The clasp actuation lines 624 can extend through and be axially movable relative to the handle 1616 and the outer shaft of the catheter assembly 1611. The clasp actuation lines 624 can also be axially movable relative to the actuation element 8102.

Referring to FIGS. 29 and 30, the width adjustment element 8211 can be releasably coupled to the coupler 8972 of the device 8200. Advancing and retracting the width adjustment element 8211 with the paddle width control 1628 widens and narrows the paddles. Advancing and retracting the actuation element 8102 with the paddle actuation control 1626 opens and closes the paddles of the device.

In the examples of FIGS. 29 and 30, the catheter or shaft of the catheter assembly 1611 is an elongate shaft extending axially between the proximal end portion 1622a, which is coupled to the handle 1616, and the distal end portion 1622b, which is coupled to the device 8200. The outer shaft of the catheter assembly 1611 can also include an intermediate portion 1622c disposed between the proximal and distal end portions 1622a, 1622b.

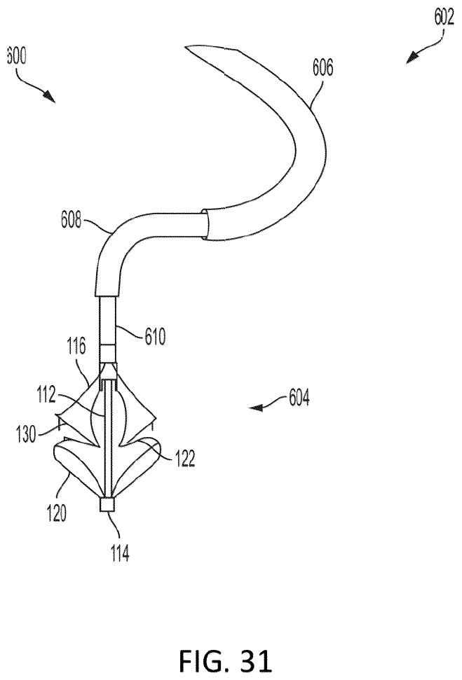

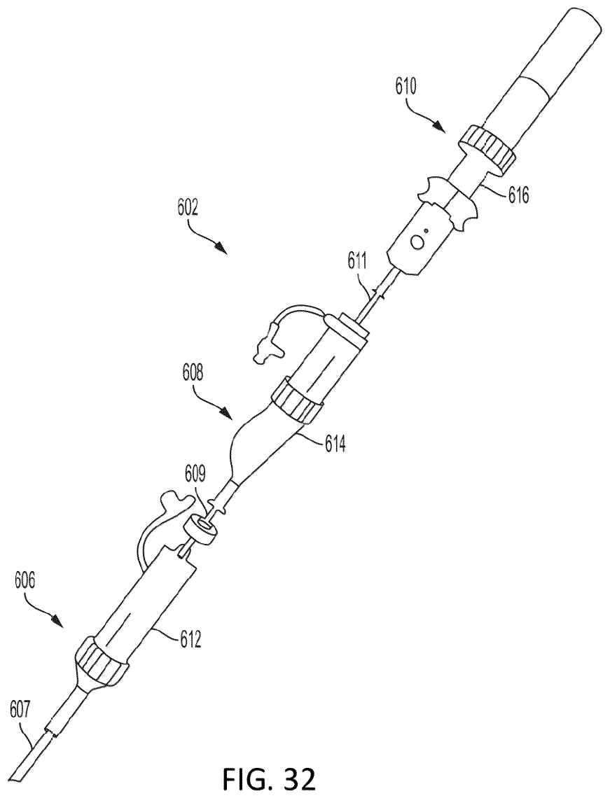

FIGS. 31-32 show an example of a system or assembly 600 (e.g., a valve treatment system or assembly, valve repair system or assembly, valve replacement system or assembly, etc.) and its components. Referring to FIG. 31, the system or assembly 600 can comprise the delivery assembly or delivery system 602 and a device 604 (e.g., a treatment device, a repair device, an implantable device, an implant, etc.). The delivery system 602 can comprise a plurality of catheter assemblies. The delivery system 602 can also comprise one or more optional catheter stabilizers or stabilizing systems/devices (not shown in FIGS. 31 and 32).

In some implementations, as shown in the illustrated example in FIG. 32, the delivery system 602 includes a first catheter assembly 606, a second catheter assembly 608, and a third catheter assembly 610. Though, in some implementation, the delivery system 602 can include fewer or more catheter assemblies than shown. In some implementations, the first catheter assembly 606 is configured as a delivery catheter assembly and will often be referred to as such for illustration herein, though it can also be other types of catheters or catheter assemblies. In some implementations, the second catheter assembly 608 is configured as a steerable catheter assembly and will often be referred to as such for illustration herein, though it can also be other types of catheters or catheter assemblies. In some implementations, the third catheter assembly 610 is configured as a device and/or implant catheter assembly and will often be referred to as such for illustration herein, though it can also be other types of catheters or catheter assemblies.

In some implementations, the second catheter assembly or steerable catheter assembly 608 extends coaxially through the first catheter assembly or delivery catheter assembly 606, and the third catheter assembly or device/implant catheter assembly 610 extends coaxially through the second catheter assembly 608 and the first catheter assembly 606. The device 604 can be releasably coupled to a distal portion of the third catheter assembly or device/implant catheter assembly 610, as further described below. It should be appreciated that the device 604 can be any device described herein.

As shown in FIG. 32, each of the catheter assemblies (e.g., delivery catheter assembly 606, the steerable catheter assembly 608, and the device/implant catheter assembly 610) includes a sheath or shaft 607, 609, 611 extending from a handle 612, 614, 616, respectively. The shaft 609 has a proximal portion 609a and a distal portion 609b The handles 612, 614, 616 are located at a proximal end of each of the corresponding sheaths or shafts, and include one or more control members to enable a user to manipulate the catheter assembly (e.g., bend or rotate the sheath or shaft of the catheter assembly) or control a component coupled to the corresponding catheter assembly (e.g., a control wire extending through the sheath or shaft of the catheter assembly).

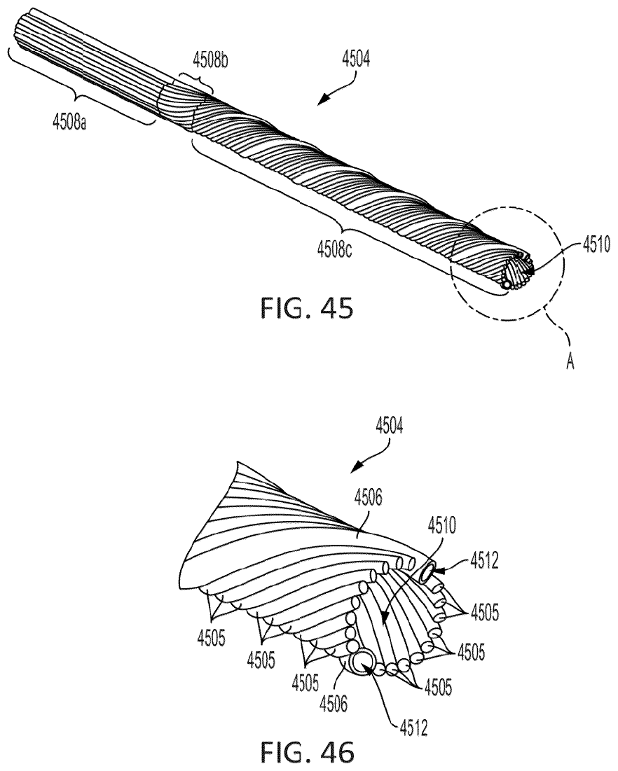

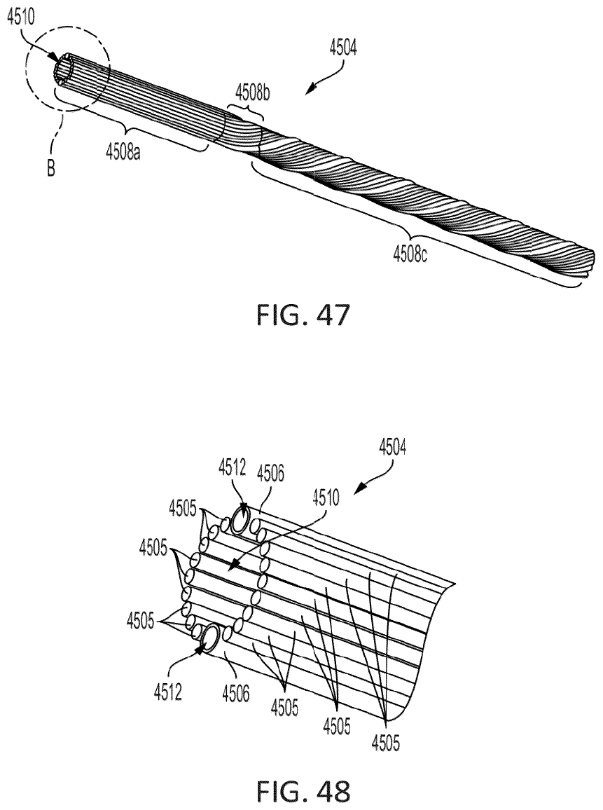

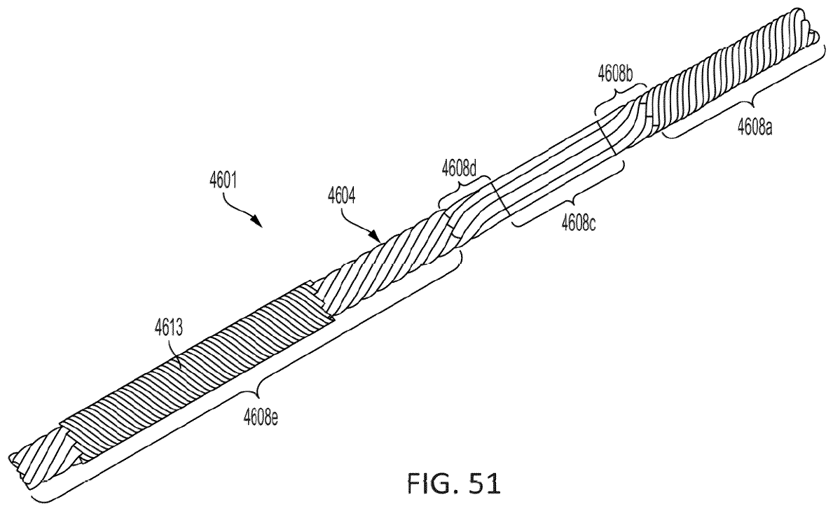

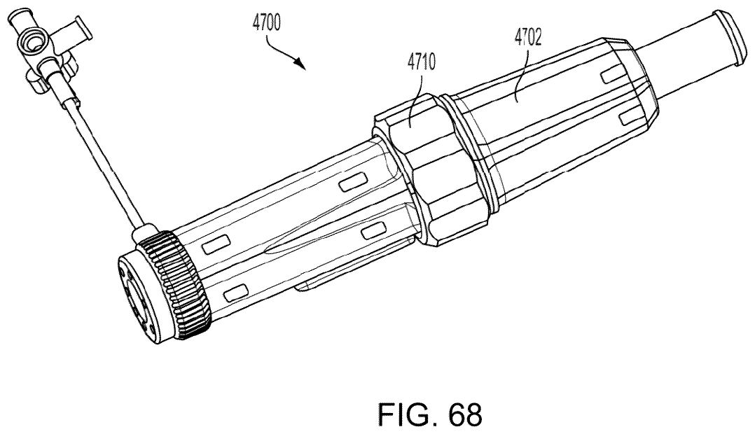

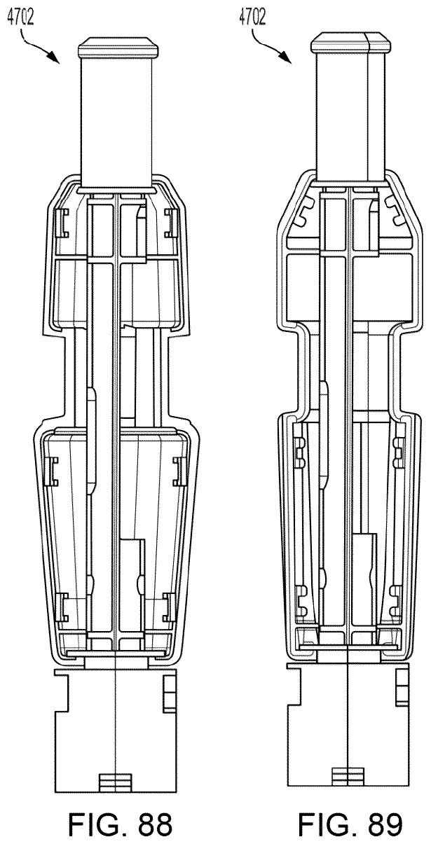

The sheaths or shafts 607, 609, 611 of each of the catheter assemblies 606, 608, 610 can take any suitable form. For example, the sheaths or shafts 607, 609, 611 can include the coil 4504 described with reference to FIGS. 45-50 or the coil 4604 described with reference to FIGS. 51-52. The handles 612, 614, 616 can take any suitable form, such as, for example, the form for the handle assembly 4700 described with reference to FIGS. 68-103.

The delivery catheter assembly 606 and the steerable catheter assembly 608 can be used, for example, to access a deployment and/or implantation location (e.g., a native mitral valve region, tricuspid valve region, or other valve region of a heart) and/or to position the device/implant catheter assembly 610 at the deployment and/or implantation location. Accordingly, in some implementations, the delivery catheter assembly 606 and the steerable catheter assembly 608 are configured to be steerable. The catheter assemblies or features of the catheter assemblies disclosed by U.S. Pat. Nos. 10,653,862 and 10,646,342 can be used as or in the catheter assemblies 606, 608, 610. U.S. Pat. Nos. 10,653,862 and 10,646,342 are hereby incorporated by reference in their entireties.

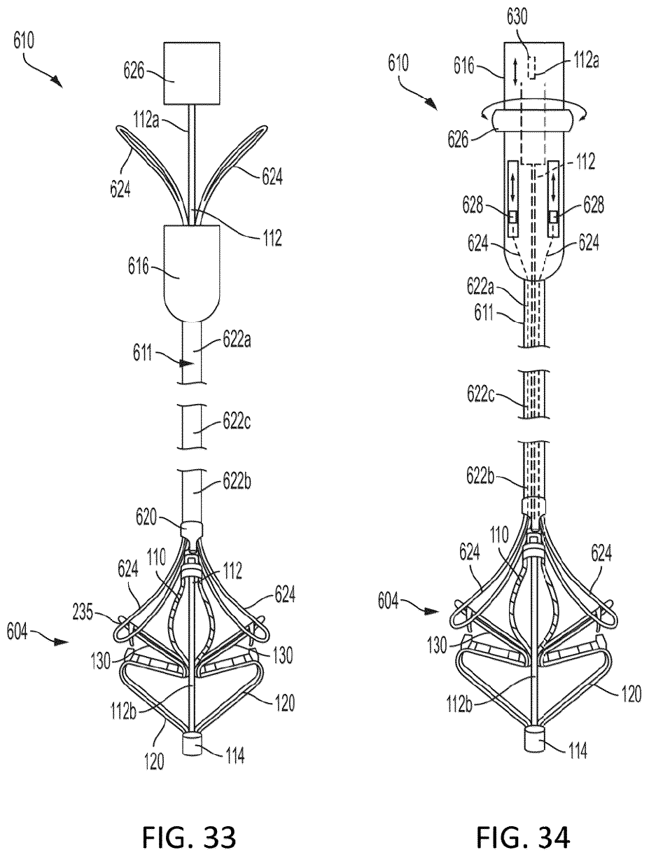

FIGS. 33 and 34 illustrate example catheter assemblies 610 (e.g., implant catheter assemblies, device catheter assemblies, treatment catheter assemblies, etc.). FIG. 33 illustrates a generalized catheter assembly 610 while FIG. 34 is a schematic illustration of an example catheter assembly 610, in which each of the clasp actuation lines 624 is coupled to a clasp control member coupled to the handle 616 and the actuation element 112 is coupled (directly or indirectly) to a control element 626 (e.g., a knob, a button, switch, slider, motor, button that controls a motor, any combination of these, which can be axially movable and/or rotatable, etc.) coupled to the handle.

In some implementations, as shown in each of the examples illustrated by FIGS. 33 and 34, the catheter assembly 610 can comprise the inner or actuation element 112, a coupler 620, an outer shaft 611, a handle 616 (shown schematically), and clasp actuation lines 624. A proximal end portion 622a of the outer shaft 611 can be coupled to extend distally from the handle 616, and a distal end portion 622b of the outer shaft 611 can be coupled to the coupler 620. The actuation element 112 can extend distally from the control element 626 (shown schematically in FIG. 81), through the handle 616, through the outer shaft 611, and through the coupler 620. The actuation element 112 can be movable (e.g., axially and/or rotationally) relative to the outer shaft 611 and the handle 616. The clasp actuation lines 624 can extend through and be axially movable relative to the handle 616 and the outer shaft 611. The clasp actuation lines 624 can also be axially movable relative to the actuation element 112.

In some implementations, the outer shaft 611 of the catheter assembly 610 can be configured to be steerable. For example, although not shown, the catheter assembly 610 can comprise an actuation element, such as a pull wire, and a flexible sleeve (e.g., a helical coil). In some implementations, the outer shaft 611 of the catheter assembly 610 is not configured to be steerable and one or more actuation elements (e.g., clasp control lines, width control element or wire, opening and closing element or wire) can be routed through a flexible sleeve (e.g., a helical coil).

As shown in FIG. 33, the actuation element 112 of the catheter assembly 610 can be releasably coupled to the cap 114 of the device 604. For example, in some implementations, the distal end portion 112b of the actuation element 112 can comprise external threads configured to releasably engage interior threads of the cap 114 of the device 604. As such, rotating the actuation element 112 in a first direction (e.g., clockwise) relative to the cap 114 of the device 604 releasably secures the actuation element 112 to the cap 114, while rotating the actuation element 112 in a second direction (e.g., counterclockwise) relative to the cap 114 of the device 604 releases the actuation element 112 from the cap 114.

In the examples of FIGS. 33 and 34, the outer shaft 611 of the catheter assembly 610 is an elongate shaft extending axially between the proximal end portion 622a, which is coupled to the handle 616, and the distal end portion 622b, which is coupled to the coupler 620. The outer shaft 611 can also include an intermediate portion 622c disposed between the proximal and distal end portions 622a, 622b. The outer shaft 611 can be formed from various materials, including metals and polymers. For example, in some implementations, the proximal end portion 622a can comprise stainless steel and the distal and intermediate portions 622b, 622c can comprise polyether block amide (PEBA). The outer shaft 611 can also comprise an outer covering or coating, such as a polymer that is reflowed over the portions 622a, 622b, and 622c.

As shown in FIGS. 33 and 34, the clasp actuation lines 624 are coupled to the clasps 130 through holes 235 in the clasps 130 or a loops or other intermediate structures attached to the clasps and extend axially through the outer shaft 611 between the clasps 130 and the handle 616. In some implementations, e.g., as illustrated by FIG. 34, at the proximal end of the clasp actuation lines 624, the clasp actuation lines 624 are each operatively and/or physically coupled to a clasp control member 628.

In some implementations, each clasp control member 628 is configured such that actuation thereof can cause axial movement of the clasp actuation line 624 relative to the handle 616, outer shaft 611 and/or the actuation element 112.

As will be described in greater detail, in some implementations, each of the clasp control members 628 can be actuated/operated independently of the other clasp control member such that each clasp actuation line 624 is moved independently relative to the handle 616, outer shaft 611, the actuation element 112, and/or the other clasp actuation line 624. In some implementations, the clasp control members 628 can be operatively or physically fixed (or synchronized) with respect to one another (e.g., locked) such that the clasp actuation lines 624 are axially moved together relative to the outer shaft 611 and the actuation element 112. In some applications, the clasp control members 628 are configured such that they can be toggled by the end user between independently actuatable and actuatable together (e.g., synchronized).

The clasp control members 628 can be configured in a variety of ways. In some implementations, one or more of the clasp control members 628 is an axially-moving control or slider coupled to a corresponding clasp actuation line 624 to axially move the clasp actuation line 624 relative to the outer shaft 611 and the actuation element 112. In some implementations, one or more of the clasp control members 628 comprises a button, switch, latch, gear, etc.

As described above, in some implementations, the actuation element 112 is coupled at a distal end to the cap 114 of the device 604. In some implementations, the actuation element 112 extends axially through the outer shaft 611 to the handle 616 and is coupled at a proximal end portion 112a to the control element 626. The actuation element 112 can be coupled to any type of control element, such as an axially moving slider or a rotational control member that is rotatable about the axis of the handle 616.

In the example illustrated by FIG. 33, the control element 626 is axially moved to axially advance or retract the actuation element, such a as a rod or wire, to open or close the device. In the example illustrated by FIG. 34, as the control element 626 is rotated about the axis of the handle 616, the rotation is translated to axial movement of the actuation element 112 and is effective to axially advance or retract the actuation element to open or close the device. Optionally, the control member can also drive an indicator component 630 between a proximal, or extended, position, and a distal, or retracted, position to indicate the position (e.g., closed, open, and extended positions) In some implementations, the control element can be a button, switch, or the like that causes a motor to rotate a shaft, gear, screw, or other component to cause axial movement of the actuation element 112.

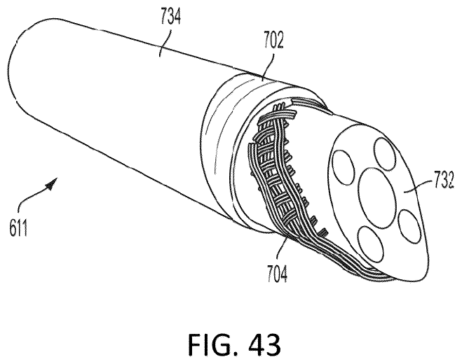

FIG. 43 is another illustration of an example device or implant catheter. As shown in FIG. 43, the catheter 611 includes stiffening material in the form of a braid, mesh, or woven material 704 and a laser-cut hypotube 702 extending over the braid, mesh, or woven material 704. As described above, the laser-cut hypotube 702 can have a variable or a constant cut pattern along a length of the device or implant catheter 611. The braid, mesh, or woven material 704 can extend over a polymer layer 732 (e.g., a PEBA layer) that defines one or more lumens of the catheter 611. In some examples, the braid, mesh, or woven material 704 can take the form of the coil 4504 described with reference to FIGS. 45-50 or the coil 4604 described with reference to FIGS. 51-52. Although illustrated in FIG. 43 as including five lumens, it is contemplated that the polymer layer 732 can include one or more lumens depending on the particular implementation. The catheter illustrated in FIG. 43 also includes a polymer jacket 734, which can impart lubricity, stiffness, or other properties to the catheter 611. One or more additional layers can also be included in some implementations, including but not limited to PTFE liners, polymer layers, adhesive layers, or the like.

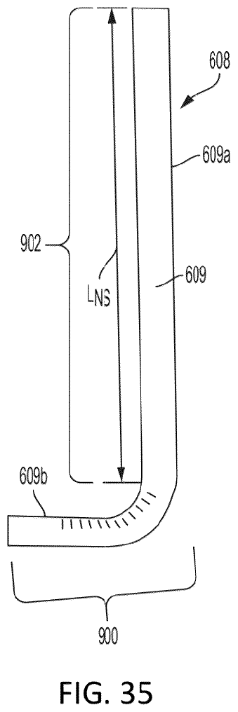

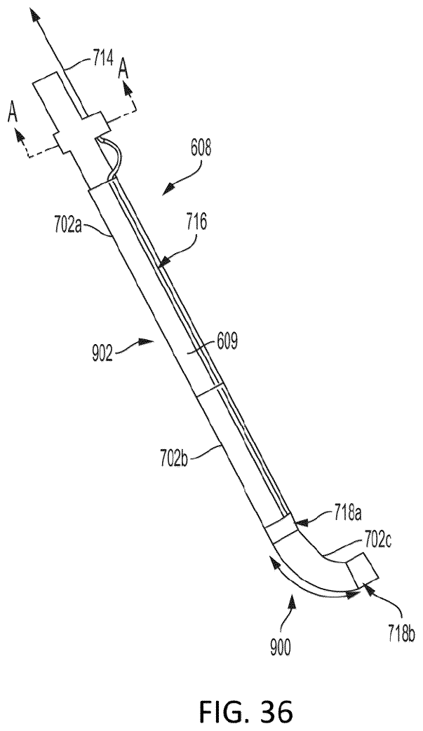

FIGS. 35 and 36 illustrate an example of a steerable catheter assembly 608. In some implementations, the steerable catheter assembly 608 includes a steerable portion 900 and a portion that is not steerable 902. In some implementations, the stiffness of the sheath 609 of the catheter assembly can vary along the length L_{NS} of the sheath. Generally, the non-steerable portion is configured to have a greater stiffness at a proximal end than at a distal end.

The stiffness of the non-steerable portion 902 can be varied in a variety of different ways. The variable stiffness non-steerable portion 902 can be used with any of the implementations disclosed by the present application or can be used in a delivery system that does not include any of the other features disclosed by the present application. The variable stiffness non-steerable portion can be used in conjunction with a decreased outer diameter of the distal portion of the sheath or shaft 611 of the catheter assembly 610 and/or a low-friction coating can be on the decreased outer diameter section of the sheath or shaft 611 of the catheter assembly 610. Further, the sheath 607 of the guide sheath or catheter assembly 606 can include a variable stiffness non-steerable portion in addition to or instead of the variable stiffness non-steerable portion of the sheath or shaft of the steerable catheter assembly 608.

As shown in FIG. 35, in some implementations, when a non-steerable portion 902 of the sheath or shaft 609 of the steerable catheter assembly 608 has a variable stiffness, such as by incorporation of a laser-cut hypotube, the variable stiffness portion can extend any length of the non-steerable portion 902. Accordingly, in some implementations, a variable stiffness length of the sheath 609 of the steerable catheter assembly 608 is less than or equal to a length L_{NS}. In some implementations, this stiffened length can include one or more stiffnesses.

A variable stiffness can be achieved in a variety of different ways. In the example illustrated by FIG. 36, a variable stiffness can be achieved, for example, by using a first laser-cut hypotube 702a along a first length of the sheath 609 of the catheter assembly 608 and a second laser-cut hypotube 702b along a second length of the sheath 609 of the catheter assembly, where the first laser-cut hypotube 702a and the second laser-cut hypotube 702b have different stiffnesses. An example catheter that includes one or more laser-cut hypotubes is disclosed by PCT Application No. PCT/US2019/062194, published as WO2020106705A1, filed on Nov. 19, 2019, which is incorporated herein by reference. Any of the features of the devices and systems disclosed by PCT Application No. PCT/US2019/062194 can be used with any of the implementations of the present application.

In FIG. 36, an actuation element, such as a pull wire 714 extends along the sheath 609 (typically embedded in the material of the sheath) of the steerable catheter assembly 608 and extends through a compression coil 716 (also typically embedded in the material of the sheath). The compression coil 716 (along with the pull wire 714) extend along (either inside or outside) the first laser-cut hypotube 702a and the second laser-cut hypotube 702b. In some implementations, each of the first laser-cut hypotube 702a and the second laser-cut hypotube 702b are formed as jackets that cover the compression coil 716. The compression coil 716 is affixed (e.g., welded or adhered) to a first ring 718a. At the first ring 718a, the pull wire 714 exits the distal end of the compression coil 716 and extends between the first ring 718a and a second ring 718b, and is affixed (e.g., welded or adhered) to the second ring 718b. In some implementations, the first ring 718a and the second ring 718b are at opposite ends of a third laser-cut hypotube 702c having a flex pattern cut into it to enable the sheath 609 of the steerable catheter assembly 608 to flex along the length of the third laser-cut hypotube 702c. Accordingly, to steer the sheath of the steerable catheter assembly 608, the pull wire 714 is pulled in a proximal direction, which causes the second ring 718b to move relative to the first ring 718a.

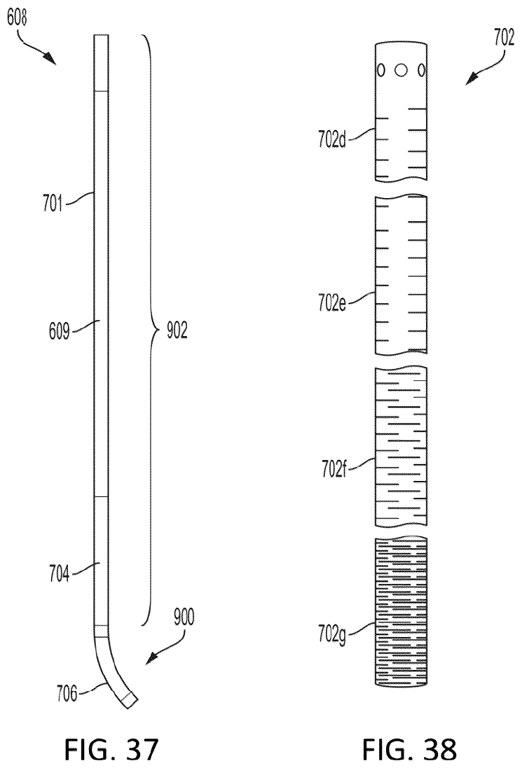

In some implementations, the sheath 609 of the steerable catheter assembly 608 illustrated in FIG. 37 or other catheter assemblies can also have a variable stiffness. In some implementations, proximal portion 701 of the sheath of the catheter assembly can comprise a laser-cut hypotube positioned over a braid (or other mesh or woven material) which surrounds the tubing or polymer material of the sheath 609 of the catheter assembly 608. In some implementations, the sheath 609 of the steerable catheter assembly 608 also includes a portion 704 in which no laser-cut hypotube is present, but the braid surrounds the tubing and a portion 706 in which the tubing is not covered by the laser-cut hypotube or the braid. Accordingly, the sheath 609 of the steerable catheter assembly 608 shown in FIG. 37 has at least three stiffnesses along its length, where each of the hypotube and the braid add to the stiffness of the tubing 706.

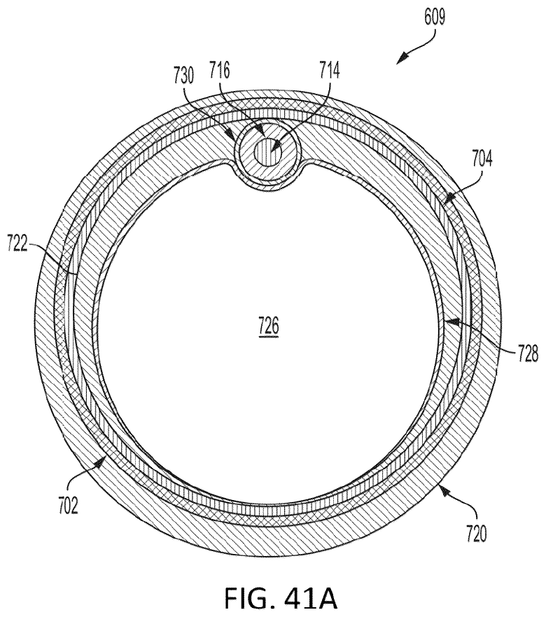

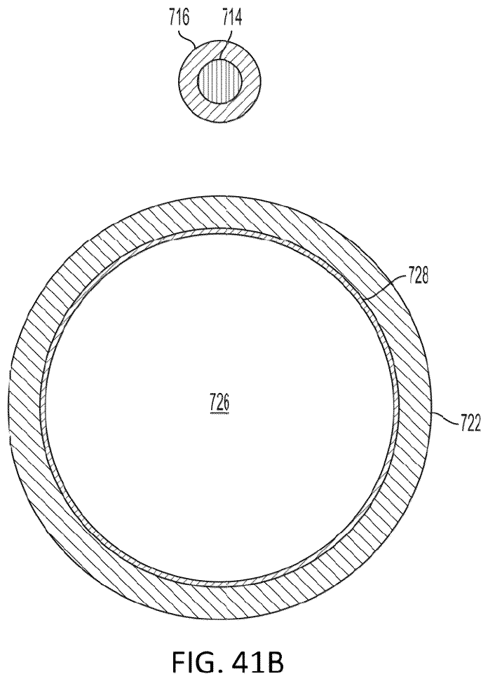

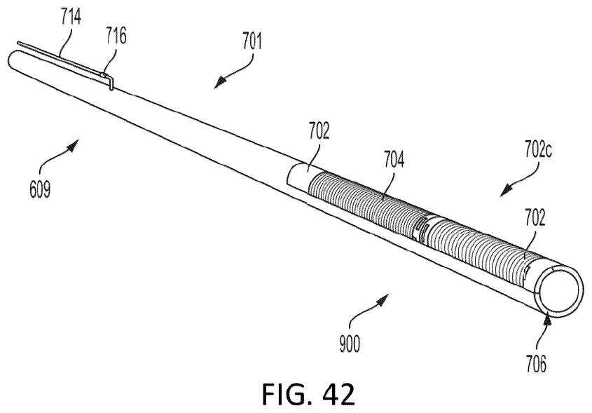

Referring to FIG. 41A and FIG. 42, in some implementations, the sheath 609 includes an elongated middle portion that comprises a braid, mesh, or woven material 704 positioned within a laser-cut hypotube 702. In some implementations, the braid, mesh or woven material 704 can take the form of the coil 4504 described with reference to FIGS. 45-50 or the coil 4604 described with reference to FIGS. 51-52. In some implementations, the braid, mesh, or woven material 704 and the laser-cut hypotube 702 are embedded in fused tubing or polymer material 706 of the sheath 609. In some implementations, as in the example illustrated by FIG. 42, the sheath 609 also includes a steerable portion 900 that comprises another laser-cut hypotube 702c. The braid, mesh, or woven material 704 and the laser-cut hypotube 702 are optionally not included in the steerable portion 900. In some implementations, the laser-cut hypotube 702c has a flex pattern cut into it to enable the sheath 609 to flex along the length of the third laser-cut hypotube 702c. Accordingly, to steer the sheath 609, the pull wire 714 is pulled in a proximal direction. Accordingly, in some implementations, the sheath 609 can include a plurality of layers that are selected to provide various properties (e.g., stiffness, lubricity, etc.) to the sheath 609 (or portions thereof) and which can extend along varying lengths of the sheath 609.

Referring to FIG. 38, in some implementations, a variable stiffness portion can be formed by a single laser-cut hypotube 702 having a variable stiffness along its length. FIG. 38 illustrates four segments (702d, 702e, 702f, 702g) of a laser-cut hypotube, each having a different cut pattern that results in different stiffness in each section. In some implementations, as in the illustrated example, the stiffness of the first segment 702d is greater than the stiffness of the second segment 702e, the stiffness of the second segment 702e is greater than the stiffness of the third segment 702f, and the stiffness of the third segment 702f is greater than the stiffness of the fourth segment 702g. In the FIG. 38 example, the uncut area of the first segment 702d is greater than the uncut area of the second segment 702e, the uncut area of the second segment 702e is greater than the uncut area of the third segment 702f, and the uncut area of the third segment 702f is greater than the uncut area of the fourth segment 702g.

The cut patterns of laser cut hypotubes can take a wide variety of different forms. In the example illustrated by FIG. 38, the first segment 702d has a pitch of about 1.27 mm (0.05 inches), 40° cut, and 51° uncut. The second segment 702e has a pitch that transitions from about 1.27 mm (0.05 inches) to about 0.5 mm (0.02 inches), the degrees cut transitions from 40° to 74° cut, and the degrees uncut transitions from 51° to 28.85° uncut. The third segment 702f has a pitch of about 0.5 mm (0.02 inches), 74° cut, and 28.85° uncut. The fourth segment 702g has a pitch that transitions from about 0.5 mm (0.02 inches) to about 0.1 mm (0.004 inches), the degrees cut transitions from 74° to 94° cut, and the degrees uncut transitions from 28.85° to 8.85° uncut. It should be understood that although four segments are illustrated in FIG. 38, the laser-cut hypotube can have any suitable number of segments having different stiffnesses. Additionally, it is contemplated that, in some implementations, the stiffness of the hypotube can decrease gradually in a distal direction (as shown in the second segment 702e and the fourth segment 702g) without defined segments.

The stiffness of each laser-cut hypotube 702 (or a segment of a hypotube) can be selected, for example, based on a material from which the hypotube is formed, a pitch of cuts, degrees of the circumference of the laser-cut hypotube that are cut, degrees of the circumference of the laser-cut hypotube that are uncut, and a kerf width of each cut. In some implementations, the hypotube is formed from and/or otherwise comprises stainless steel or nitinol, although other materials are suitable and contemplated.

FIG. 39 illustrates a hypotube 702 having an interrupted spiral cut and shown as a rectangular sheet. In FIG. 39, the circumference of the hypotube is represented by the letter C and the pitch is represented by the letter P. The degrees cut 708 is a length of each cut, and the degrees uncut 710 is a distance between one cut and an adjacent cut in the direction of the circumference C. The kerf width 712 of each cut refers to the width of the space between two adjacent laser cuts at a point at which the sides of the cut are parallel to one another.

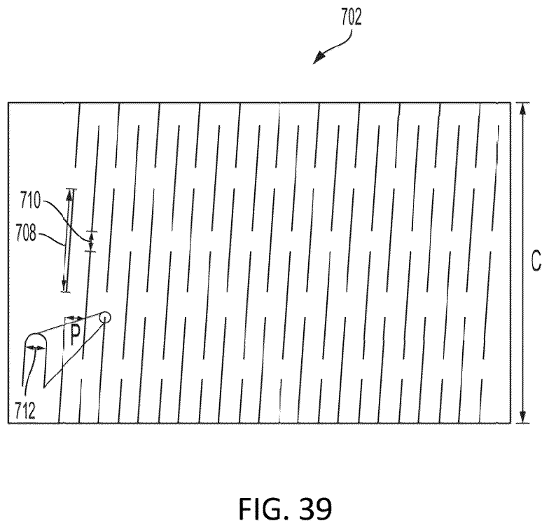

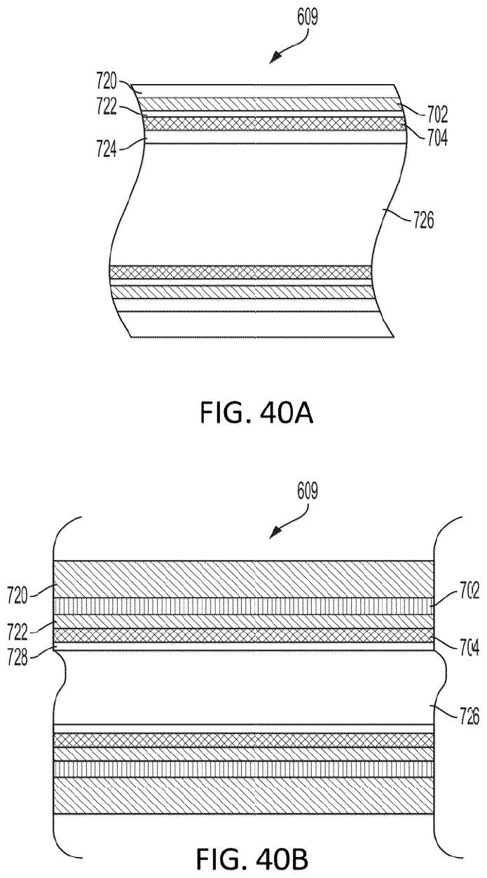

As described above, in some implementations, stiffening materials can be incorporated in the form of a jacket of the sheath 609 of the steerable catheter assembly 608, or the stiffening materials can be incorporated into the structure of the sheath as layers of a multi-layer sheath. Example multi-layer sheaths are shown in FIGS. 40A, 40B, 41A, and 41B. The number and type of layers can vary from what is shown. The layers can be extruded, molded, or otherwise formed and combined in a variety of ways.

FIG. 40A is a longitudinal cross-section of an example multi-layer sheath that includes (from the outer surface of the multi-layer sheath toward the lumen) a first polymer layer 720, a laser-cut hypotube 702, a second polymer layer 722, a braid, mesh, or woven material 704, a third polymer layer 724 defining the lumen 726. Similarly, the multi-layer sheath in FIG. 40B includes (from the outer surface of the multi-layer sheath toward the lumen) the first polymer layer 720, the laser-cut hypotube 702, the second polymer layer 722, the braid, mesh, or woven material 704, and the lumen 726.

In some implementations, as in the example illustrated in FIG. 40B, a polytetrafluoroethylene (PTFE) liner 728 defines the lumen 726. As previously described, the use of a PTFE liner 728 can be used to reduce friction along the internal diameter of the lumen 726. The polymer layers 720, 722, 724 can provide torque resistance based on the durometer of the polymer used, and the laser-cut hypotube 702 and the braid, mesh, or woven material 704 can further provide torque resistance to the multi-layer sheath. It is contemplated that a cross-section of the multi-layer sheath can vary along the length of the sheath. For example, as described herein, the laser-cut hypotube 702, the braid 704, or both can be present in proximal portions of the sheath, but not in distal portions of the sheath to provide a variable stiffness of the sheath.

FIG. 41A illustrates a radial cross-section of an example multi-layer sheath. For example, the cross-section illustrated by FIG. 41A can correspond to one or more portions of the sheath (e.g., to a middle portion of the sheath, etc.) illustrated by FIG. 42. The multi-layer sheath example shown in FIG. 41A includes two lumens. For example, when the multi-layer sheath is used as a sheath 609 of a steerable catheter assembly 608, the shaft 611 of the catheter assembly 610 can pass through the lumen 726, and the control element, such as the illustrated pull wire 714, and compression coil 716 can extend through a second lumen, as described above with respect to FIG. 36. In the example shown in FIG. 41A, the lumen 726 is defined by a PTFE liner 728, which is positioned within the second polymer layer 722.

In some implementations, a braid, mesh, or woven material 704 is positioned around the second polymer layer 722 and is positioned within the laser-cut hypotube 702. In some implementations, the first layer of polymer 720 surrounds the laser-cut hypotube 702. Within the second polymer layer 722 and adjacent to the PTFE liner 728, a second liner 730 defines the second lumen, through which the control element 714 and compression coil 716 extend. It should be appreciated that other layers and other lumens can be present in some implementations, and the layers can be provided in alternative orders while providing the torque resistance described herein.

FIG. 41B illustrates a radial cross-section of an example multi-layer sheath in which the control element 714 and coil are on the outside of the proximal portion of the sheath. The cross section can correspond to various locations along the length of the sheath. For example, in some implementations, the cross-section illustrated by FIG. 41B corresponds to a proximal portion of the sheath illustrated by FIG. 42. In some implementations, the second lumen for accommodating the control element runs less than the full length of the multi-layer sheath.

In some implementations, the lumen of the multi-layer sheath varies along the length of the multi-layer sheath and, for example, can transition from a circular cross-section to a non-circular cross-section along the length of the multi-layer sheath. The cross-section illustrated in FIG. 41B can correspond to a proximal portion of the multi-layer sheath relative to the radial cross-section illustrated in FIG. 41A, such as along the line A-A in FIG. 36. As shown in FIGS. 41B and 42, the control element 714 is external to the multi-layer sheath, and the lumen 726 is defined by a PTFE liner 728, which is positioned within the second polymer layer 722.

In some implementations, the braid 704 (or other mesh or woven material), the laser-cut hypotube 702, and the first layer of polymer 720 that surrounds the laser-cut hypotube 702 do not extend along the proximal length of the multi-layer sheath, enabling the control element 714 to enter the second lumen of the multi-layer sheath as described above. The lumen 726 has a substantially circular cross-section. In some implementations, a groove in the mandrel used to support the PTFE liner 728 and the second polymer layer 722 is filled at a position corresponding to the proximal end of the multi-layer sheath to enable the lumen 726 illustrated in FIG. 41B to be formed. In some of these implementations, reflow of the PTFE and/or the polymer used to form the second polymer layer can create a gradual transition between a circular cross-section and the non-circular cross-section illustrated in FIG. 41A. It should be appreciated that other layers and other lumens can be present in some implementations, and the layers can be provided in alternative orders while providing the torque resistance described herein.

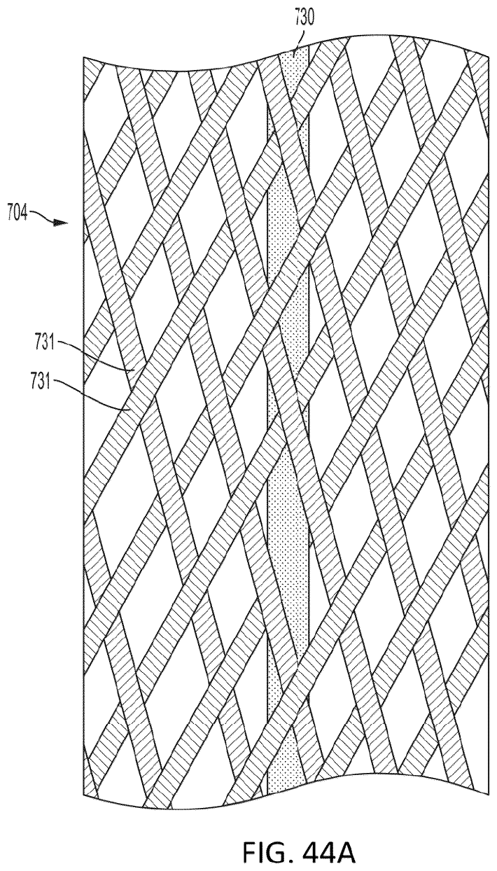

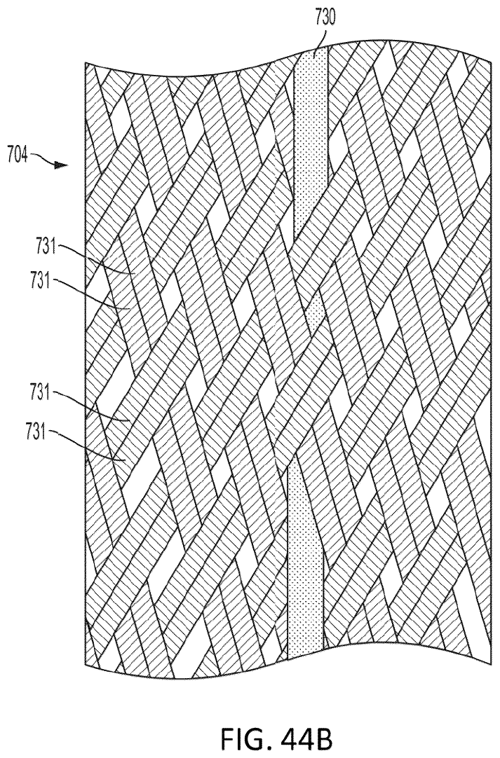

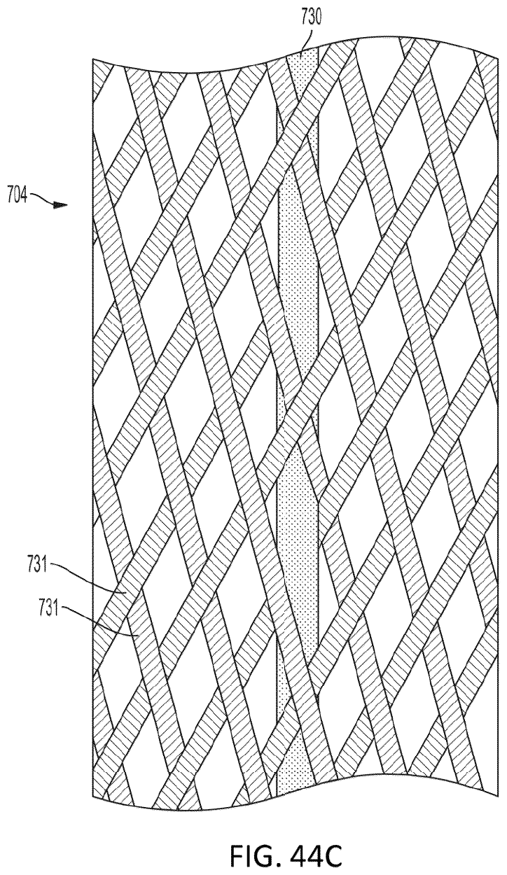

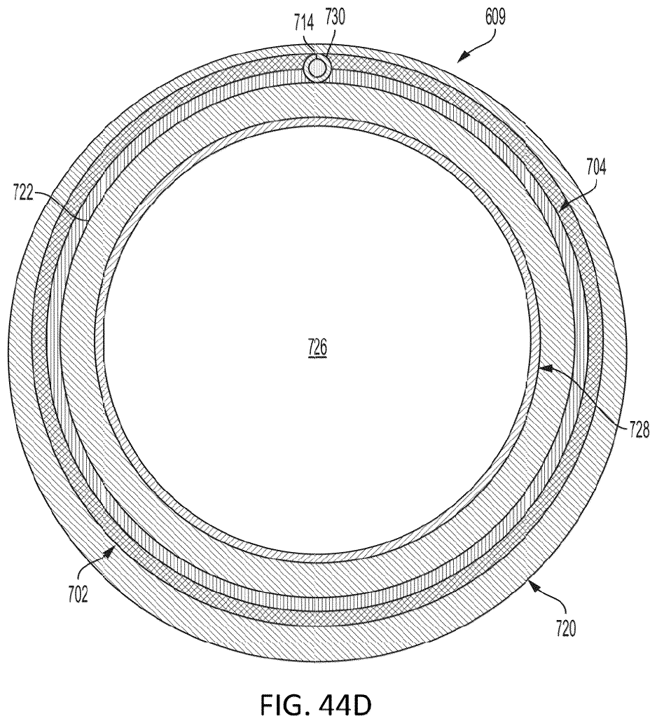

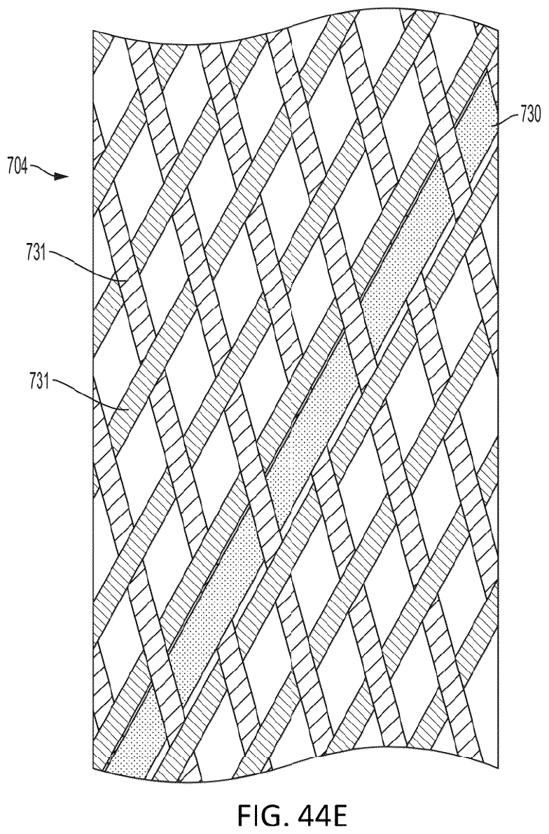

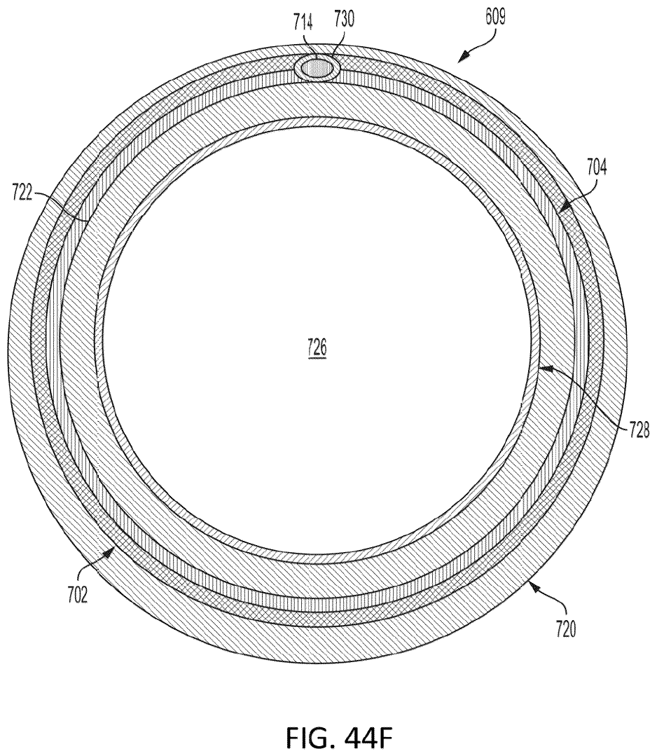

In some implementations, the braid of the multi-layer sheath can be used to facilitate a passage for a control element, such as a steering element (e.g., wire), clasp control line, paddle control element (for opening and closing the paddles), a width control element (for adjusting the width of the paddles), etc. The incorporation of the passage into the braid can enable the separate lumen (lumen 726) under the braid, and, in some implementations, the compression coil 716, to be removed, which can in turn reduce the outer diameter profile of the multi-layer catheter (implant catheter, device catheter, steerable catheter, guide sheath, etc.), allow for a more uniform (e.g., circular, etc.) profile, allow for a larger inner profile, and/or reduce manufacturing complexity. Various braid patterns incorporating a control element passage are illustrated in FIGS. 44A-44C and 44E.

In FIGS. 44A-44C, a lumen 730 is woven into the braid 704, with the lumen 730 extending longitudinally or along the direction of the length of the sheath. The lumen 730 can extend along the length of the sheath in a variety of different ways. For example, the lumen 731 can extend longitudinally and wires 731 of the braid are disposed at two opposing angles relative to the lumen. In some implementations, the lumen 730 and the other wires form a triaxial braid. In some implementations, the lumen 730 and the wires 731 of the braid form sixty-degree angles or about sixty-degree angles relative to one another.

In some implementations, the lumen 730 can be a tube, such as a thin-walled tube, made from any suitable material. In some implementations, the lumen 730 can be a stainless steel or nitinol hypotube. In some implementations, the lumen 730 can be a polymeric tube, such as a tube made from polyamide, PEEK, or other polymers known and used in the art. The lumen can be made from any material. In the implementations illustrated in FIGS. 44A-44C, the lumen 730 is incorporated into the braid structure and runs longitudinally along the length of the braid 704. While providing the passage for the control element, e.g., pull wire 714, the lumen 730 can further reinforce the braid and strength of the multi-layer sheath.

In some implementations, the lumen 730 is braided in and out of every other crossing or pick of the braid 704, although other patterns are contemplated and possible. As illustrated in FIGS. 44A-44C, the lumen 730 can be incorporated into any one of a variety of braid patterns, such as tri-axial braid patterns or any pattern where the lumen extends longitudinally. For example, FIG. 44A illustrates the longitudinally extending lumen 730 incorporated in a full braid pattern, FIG. 44B illustrates the longitudinally extending lumen 730 incorporated in a diamond braid pattern, and FIG. 44C illustrates the longitudinally extending lumen 730 incorporated in a half diamond braid pattern.

FIG. 44D illustrates a radial cross-section of an example multi-layer sheath in which the control element, such as the illustrated pull wire 714, or a clasp control element, a paddle control element, width control element, etc., passes through a longitudinally extending lumen 730 that is woven into the braid 704. The control element 714 and the lumen 730 can be used in the same manner as the control element and the compression coil 716 described above to flex or steer the sheath or steerable catheter and/or control one or more of the moving parts of a treatment and/or repair device. For example, application of tension to the control element 714 in the lumen 730 causes a portion of the sheath to flex. The cross-section can correspond to various locations along the length of the sheath. For example, in some implementations, the cross-section illustrated by FIG. 44D corresponds to a proximal portion of the sheath illustrated by FIG. 42. In some implementations, the lumen 730 for accommodating the control element runs less than the full length of the multi-layer sheath.

In some implementations, the braid 704 (or other mesh or woven material), the laser-cut hypotube 702, and the first layer of polymer 720 that surrounds the laser-cut hypotube 702 do not extend along the proximal length of the multi-layer sheath, enabling the control element 714 to enter the lumen 730 of the multi-layer sheath as described above. The lumen 730 can have a substantially circular cross-section or a non-circular cross-section. It should be appreciated that other layers and other lumens can be present in some implementations, and the layers can be provided in alternative orders while providing the torque resistance described herein.

In the example shown in FIG. 44E, the lumen 730 is woven into the braid 704 such that the lumen does not extend only longitudinally or only in the direction of the length of the sheath. For example, the lumen 730 can wrap or twist about the sheath as the lumen extends along the length of the sheath (e.g., in a helical configuration). In some braids, chase wires that extend along or parallel with another wire of the braid are included. In the implementation illustrated by FIG. 44E, the lumen 730 replaces a chase wire in the braid.

As above, the lumen 730 can be a tube, such as a thin-walled tube, made from any suitable material. In some implementations, the lumen 730 can be a stainless steel or nitinol hypotube. In some implementations, the lumen 730 can be a polymeric tube, such as a tube made from polyamide, PEEK, or other polymers known and used in the art. The lumen 730 can be made from any material. In the implementations illustrated in FIG. 44E, the lumen 730 is braided into the braid 704 next to another wire of the braid. While providing the passage for the control element, such as the illustrated pull wire, the lumen 730 can further reinforce the braid and strength of the multi-layer sheath. As a chase wire-type feature, the lumen 730 can reduce bowing of the multi-layer sheath during application of tension to the control element, as it spirals around the multi-layer sheath. In implementations, the lumen 730 is incorporated as a chase wire feature in a half diamond braid pattern. However, the lumen 730 can have any spiral or helical configuration and can be incorporated into any braid pattern.

FIG. 44F illustrates a radial cross-section of an example multi-layer sheath in which the control element 714 passes through a spiraled, helical, or otherwise winding lumen 730 that is woven into the braid 704. The cross sections of the lumen 730 and control element 714 are non-circular (e.g., elliptical or otherwise shaped), since the section extends through the spiraled, helical, or otherwise winding control element, such as the illustrated pull wire 714, and lumen 730. The cross section can correspond to various locations along the length of the sheath. For example, in some implementations, the cross-section illustrated by FIG. 44F corresponds to a proximal portion of the sheath illustrated by FIG. 42. In some implementations, the lumen 730 for accommodating the control element runs less than the full length of the multi-layer sheath.

In some implementations, the braid 704 (or other mesh or woven material), the laser-cut hypotube 702, and the first layer of polymer 720 that surrounds the laser-cut hypotube 702 do not extend along the proximal length of the multi-layer sheath, enabling the control element 714 to enter the lumen 730 of the multi-layer sheath as described above. In contrast to the lumen illustrated in FIG. 44D, the lumen 730 illustrated in FIG. 44F has a substantially elliptical cross-section due to its coiled configuration (e.g., not longitudinally extending) within the braid 704. It should be appreciated that other layers and other lumens can be present in some implementations, and the layers can be provided in alternative orders while providing the torque resistance described herein.

FIGS. 45-50 illustrate an example of a coil 4504 for a multi-layer sheath 4501 of a catheter assembly. The coil 4504 can be used with a sheath of any suitable catheter assembly of a system for delivering a device, such as with a catheter assembly of any system for delivering a device described in the present application. For example, the coil 4504 can be used with any delivery catheter assembly, steerable catheter assembly, and/or device/implant catheter assembly described in the present application.

Referring to FIGS. 46 and 48, the coil 4504 can include one or more supporting elements 4505 (e.g., wires, fibers, strands, extrusions, etc.) and one or more tubes 4506, wherein the supporting elements 4505 and tubes 4506 are coiled together to create the composite coil 4504 that defines a main lumen 4510. Each of the tubes 4506 can include a lumen 4512 such that the coil 4504 has one or more lumens 4512 that surround the main lumen 4510. In the illustrated example, the coil 4504 includes eighteen supporting elements 4505 and two tubes 4506. However, it should be understood that the coil can include any suitable number of supporting elements 4505 and any suitable number of tubes 4506. For example, the composite coil 4504 can include one or more lumens, two or more lumens, three or more lumens, four or more lumens, five or more lumens, six or more lumens, seven or more lumens, eight or more lumens, nine or more lumens, ten or more lumens, etc. The number of supporting elements 4505 and/or tubes 4506 can be based on the desired size of the main lumen 4510.

The tube(s) 4506 can be any suitable tube member, such as a thin-walled tube, made from any suitable material. In some implementations, the tube(s) 4506 can be a stainless steel or nitinol hypotube. In some implementations, the tube(s) 4506 can be a polymeric tube, such as a tube made from polyamide, PEEK, or other polymers known and used in the art. The tube(s) 4506 can be made from any suitable material.

In some implementations, the coil 4504 can include multiple segments such that each segment has different characteristics. In the illustrated example, the coil 4504 includes three segments (4508a, 4508b, 4508c). In some implementations, the first segment 4508a is a straight portion (i.e., not coiled, only extending longitudinally) that is configured to provide tensile strength and bendability to the coil 4504. The first segment 4508a can be disposed at a proximal end of the coil 4504. In some implementations, the third segment 4508c can be a curved portion that includes a pitch angle of between 2 degrees and 80 degrees, such as between 10 and 70 degrees, such as between 20 and 60 degrees, such as between 25 and 50 degrees. The curved portion of the third segment 4508c can be configured to have compressive strength (e.g., the pitch angle can be low, such as between 2 degrees and 20 degrees.

In some implementations, the supporting elements 4505 and tubes 4506 of can be coiled in a clockwise or counterclockwise direction in the curved region, which allows for a unidirectional torque resistance in the direction of coiling. The third segment 4508c can be disposed at a distal end of the coil 4504. The second segment 4508b is a transition portion as the tube(s) 4506 and/support elements 4505 move from the straight portion to the curved portion. While the coil 4504 is shown as having three segments, it should be understood that the coil 4504 can have any suitable number of segments. It should also be understood that these segments can be straight portions or curved portions based on the desired characteristics of the segment.

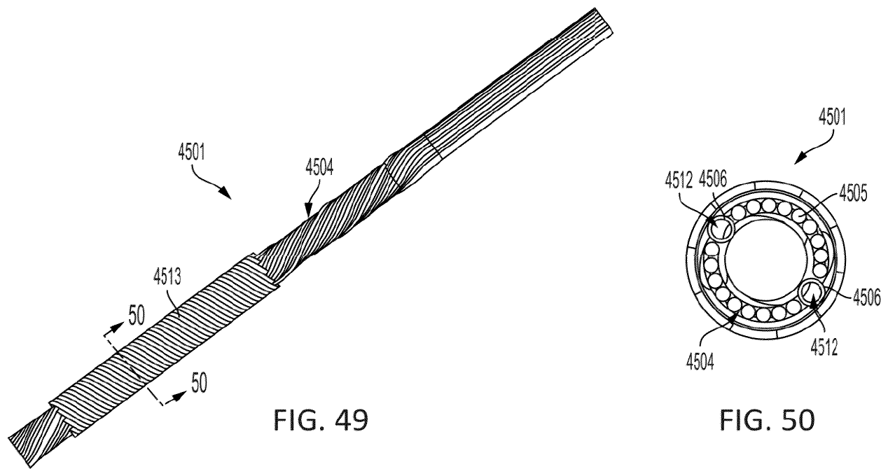

Referring to FIGS. 49-50, the multi-layer sheath 4501 can include an outer coil layer 4513 that is disposed over the coil 4504. The outer coil layer 4513 can include a plurality of support members (e.g., wires, fibers, strands, laser cut hypotube portions, etc.) that are coiled in a clockwise or counterclockwise direction, which allows for a unidirectional torque resistance in the direction of coiling. In some implementations, the direction of coiling of the outer coil layer 4513 is opposite to the direction of coiling for the third segment 4508c of the coil 4504 such that the multi-layer sheath 4501 has torque resistance in each direction. The outer coil layer 4513 can have a pitch angle of between 2 degrees and 80 degrees, such as between 10 and 70 degrees, such as between 20 and 60 degrees, such as between 25 and 50 degrees. The outer coil layer 4513 can have a pitch angle that allows for the outer coil layer 4513 to be compression and kink resistant.

FIGS. 51-52 illustrate an example of a coil 4604 for a multi-layer sheath 4601 of a catheter assembly. The coil 4604 can be used with a sheath of any suitable catheter assembly of a system for delivering a device (e.g., treatment device, repair device, implantable device, implant, etc.), such as with a catheter assembly of any system for delivering a device or implant described in the present application. For example, the coil 4604 can be used with any delivery catheter assembly, steerable catheter assembly, and/or device/implant catheter assembly described in the present application.

In some implementations, the coil 4604 can include a plurality of tubes 4606 that are coiled together to define a main lumen 4610. In some implementations, each of the tubes 4606 can include a lumen 4612 such that the coil 4604 has one or more lumens 4612 that surround the main lumen 4610. In the illustrated example, the coil 4604 includes eight tubes 4606. However, it should be understood that the coil can include any suitable number of tubes 4606. For example, the coil 4604 can include three or more lumens, four or more lumens, five or more lumens, six or more lumens, seven or more lumens, eight or more lumens, nine or more lumens, ten or more lumens, etc. The number of tubes 4606 can be based on the desired size of the main lumen 4610. While the illustrated example does not show the coil 4604 having any supporting elements (e.g., the supporting elements 4505 shown in FIGS. 45-50), it should be understood that the coil 4604 can include supporting element(s) that are coiled together with the plurality of tubes 4606 to create the main lumen 4610.

The tube(s) 4606 can be any suitable tube member, such as a thin-walled tube, made from any suitable material. In some implementations, the tube(s) 4606 can be a stainless steel or nitinol hypotube. In some implementations, the tube(s) 4606 can be a polymeric tube, such as a tube made from polyamide, PEEK, or other polymers known and used in the art. The tube(s) 4606 can be made from any suitable material.

In some implementations, the coil 4604 can include multiple segments such that each segment has different characteristics. In the illustrated example, the coil 4604 includes five segments (4608a, 4608b, 4608c, 4608d, 4608e). In some implementations, the first segment 4608a is a curved portion that is configured to provide compression strength to the coil 4604 due to a high pitch angle. The first segment 4608a can be coiled in a clockwise or counterclockwise direction, which allows for a unidirectional torque resistance in the direction of coiling. In some implementations, the pitch angle of the first segment 4608a can be between 2 degrees and 80 degrees, such as between 10 and 70 degrees, such as between 20 and 60 degrees, such as between 25 and 50 degrees. The first segment 4608a can be disposed at a proximal end of the coil 4604. The third segment 4608c can be a straight portion that is configured for tensile strength and bendability to the coil 4604. The fifth segment 4608e is a curved portion having a pitch angle of between 2 degrees and 80 degrees, such as between 10 and 70 degrees, such as between 20 and 60 degrees, such as between 25 and 50 degrees. The curved portion of the fifth segment 4608e can be coiled in the clockwise or counterclockwise direction. The fifth segment 4608e can be coiled in a clockwise or counterclockwise direction, which allows for a unidirectional torque resistance in the direction of coiling.

In some implementations, the second segment 4608b is a transition portion as the tube(s) 4506 move from the first segment 4608a to the third segment 4608c, and the fourth segment 4608d is a transition portion as the tube(s) 4506 move from the third segment 4608c to the fifth segment 4608e. While the coil 4604 is shown as having five segments, it should be understood that the coil 464 can have any suitable number of segments. It should also be understood that these segments can be straight portions or curved portions based on the desired characteristics of the segment.

In some implementations, the multi-layer sheath 4601 can include an optional outer coil layer 4613 that is disposed over the coil 4604. In some implementations, the outer coil layer 4613 can include a plurality of support members (e.g., wires, strands, fibers, tubes) that are coiled in a clockwise or counterclockwise direction, which allows for a unidirectional torque resistance in the direction of coiling. In some implementations, the direction of coiling of the outer coil layer 4613 is opposite to the direction of coiling for at least one of the first segment 4608a and the fifth segment 4608e of the coil 4604 such that the multi-layer sheath 4601 has torque resistance in each direction. In some implementations, the outer coil layer 4613 can have a pitch angle of between 2 degrees and 80 degrees, such as between 10 and 70 degrees, such as between 20 and 60 degrees, such as between 25 and 50 degrees. In some implementations, the outer coil layer 4613 can have a pitch angle that allows for the outer coil layer 4613 to be compression and kink resistant.

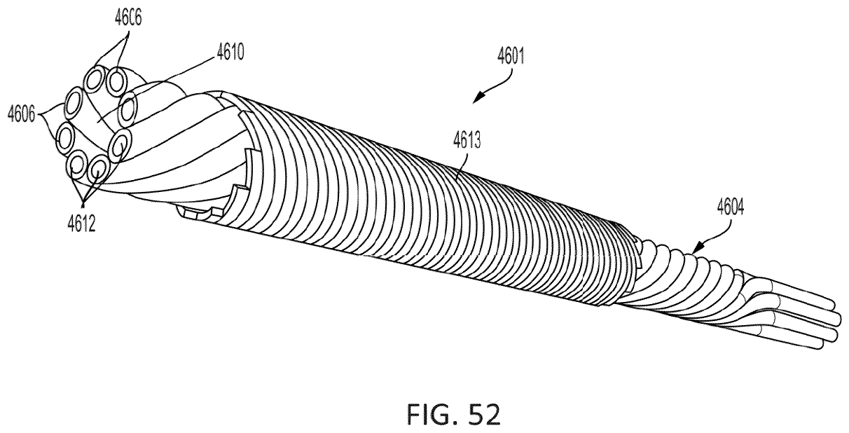

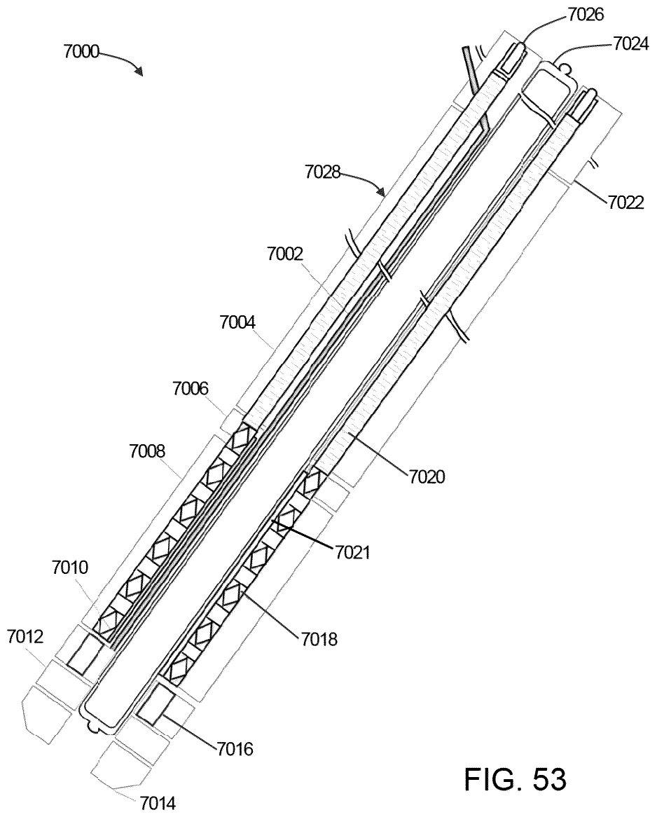

FIGS. 53-67 illustrate examples of a multi-layer sheath or catheter systems having axial, flexural and/or torque response. Axial response is provided by column strength in the sheath to aid in, for example, insertion and pushability (e.g., for septal puncture). Flexibility response is provided to navigate a patient's anatomy and also aid in septum puncture. Torsional response is provided to support the implant delivery and manipulation. In the various implementations, a multi-layer sheath or catheter system having a tube-based design, which can be a hypotube, is provided. This can reduce the sheath profile size compared to braided and/or coiled structures. It also simplifies manufacturing because less durometers are needed in order to obtain the flexural properties of the sheath because a hypotube can be tuned. Manufacturing improvements are also provided in that hypotube designs are less complex to manufacture and assemble compared to braided and/or coiled structures. This can result in higher manufacturing yields.

In some implementations, a multi-lumen sheath or catheter system in provided that includes, for example, a patterned hypotube, a multi-lumen inner liner, and one or more polymer outer liners (or jackets). The patterned hypotube can include a plurality of patterns including for example, a first pattern in a proximal portion of the tube and a second pattern in a distal portion of the tube. The first pattern can include a spiral cut pattern (including an interrupted spiral cut pattern(s)) to provide, for example, torque and/or compression resistance to the tube. The second pattern can include one or more cuts forming flex windows or cuts that provide the tube with flexure, while for example minimizing impact on the sheath column profile or straightness. The patterns can also include reflow ports or windows that provide openings for polymer (or similar material) insertion that can form, for example, various durometer portions along the sheath. The pattern of flex windows can also be arranged to prevent/minimize tube inside diameter constriction during flex. Each pattern can include one or more additional patterns (or sub-patterns) for providing flexure control, reflow, and/or connectivity/attachment. Additional patterns can be provided in the hypotube to provide, for example, transitions in flexure or stiffness between two or more portions of the hypotube (e.g., between the proximal portion and distal portion). By patterning the hypotube appropriately, desire flex profiles of the sheath can be obtained while also limiting undesirable flex to achieve clinically relevant articulation of the sheath/catheter without causing harm to the patient.

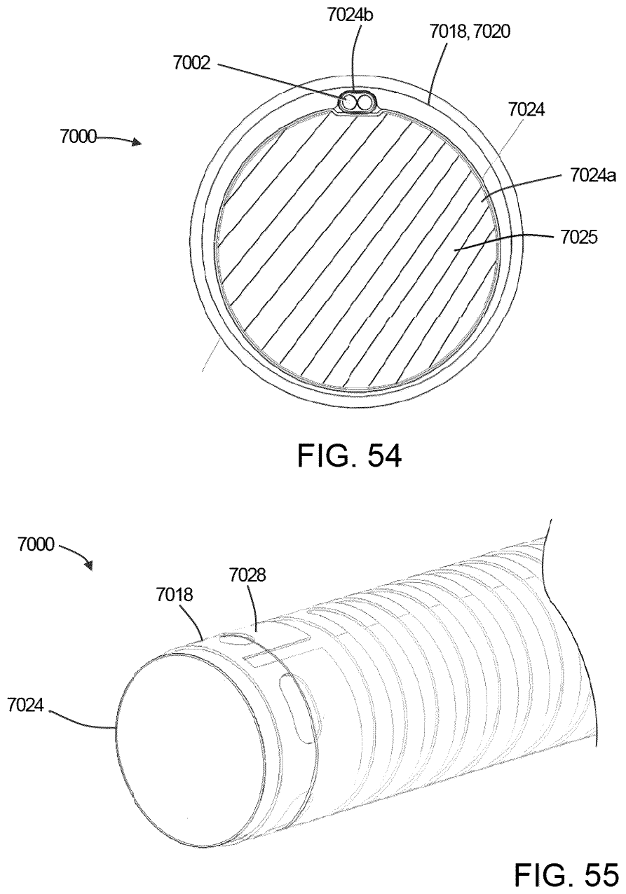

FIG. 53 illustrates an example of a multi-layer sheath system 7000. System 7000 includes, for example, a tube that can be a hypotube having a distal tube portion 7018 and a proximal tube portion 7020. As described above, each of these tube portions can be patterned and have different structural properties including, for example, axial, flexural and torque response. An inner liner portion 7024 (with an optional removable mandrel 7025 (FIG. 54) used during manufacture) is provided inside the tube that includes one or more lumens. One of the lumens can carry within it one or more wire(s) or actuation element(s) 7002. System 7000 further includes a pull ring 7016 at the distal end portion of the tube and one or more ears, fingers or other projections 7026 at the proximal end portion of the tube.

The multi-layer sheath system 7000 includes an outer jacket 7028 or layer. The outer jacket 7028 can include different durometer portions, including first durometer portion 7004, second durometer portion 7006, third durometer portion 7008, fourth durometer portion 7010, fifth durometer portion 7012, a tip or sixth durometer portion 7014, and seventh durometer portion 7022. Each of these durometer portions can have one or more differing hardness or softness values. For example, first durometer portion 7004 can have a value of 55 D. Second durometer portion 7006 can have a value of 45 D. Third durometer portion 7008 can have a value of 35 D. Fourth durometer portion 7010 can have a value of 35 D. Fifth durometer portion 7012 can have a value of 35 D. Tip or sixth durometer portion 7014 can have a value of 35 D or less. And, seventh durometer portion 7022 can have a value of 55 D. Other durometer values can also be used, including those varying +/−1-50%, 2-20%, 3-10%, 4-5%, or any sub range of these ranges from the durometers stated above for each portion. While example durometer values have been provided, other values can be used based on the desired hardness or softness and the overall hypotube design structural characteristics. The various durometer portions of the outer jacket 7028 can be made from, for example, a thermoplastic elastomer made of polyamide (rigid) and/or ether blocks (elastomeric). Other materials can also be used.

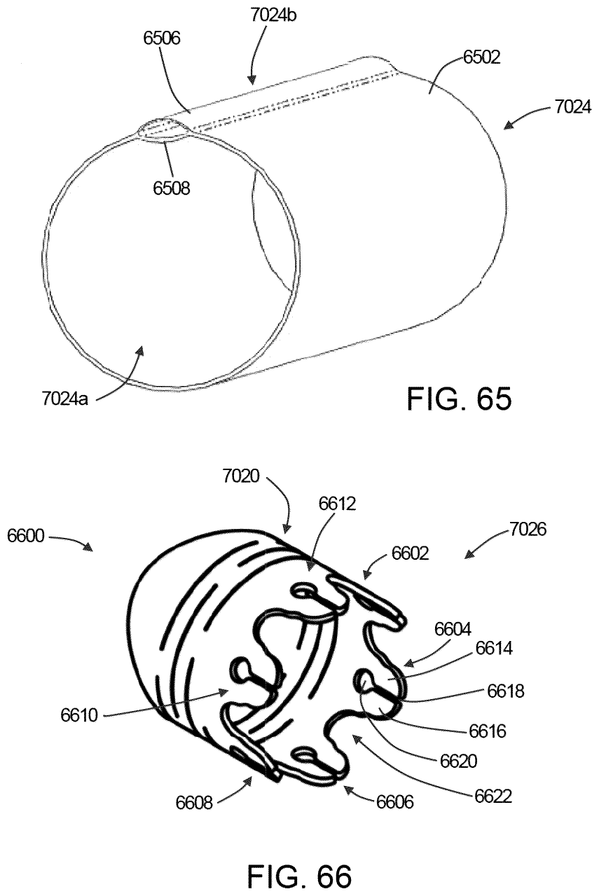

FIG. 54 is a cross-sectional schematic view of components of the multi-lumen sheath system shown in FIG. 53 and a manufacturing mandrel 7024a. Inner liner portion 7024 can have a multi-lumen arrangement that includes, for example, central lumen 7024a and perimeter lumen 7024b. See also FIG. 65 showing a partial perspective view of inner liner 7024 having liner body 6502 having first portion 6506 forming main lumen 7024a and second portion 6508 forming perimeter lumen 7024b. Lumen 7024b can include one or more wires or actuation element(s) 7002. Inner liner portion 7024 is positioned inside the proximal tube portion 7020 and distal tube portion 7018.

Referring to FIGS. 53 and 55, an outer jacket or layer 7028 is provided, which can include one or more durometer portions (including those previously described). Inner liner portion 7024 can be separated from and/or bonded to the tube portions by one or more tie layers 7021 or portions. The tie layer 7021 can take a variety of different forms. In the example illustrated by FIG. 53, the tie layer 7021 extends only along the portion 7018 of the hypotube. In other implementations, the tie layer 7021 can extend along the entire tube, along the portion 7020, along a portion of the portion 7018, along a portion of the portion 7020, or along portions of both the portion 7018 and the portion 7020. Also, inner liner portion 7024 can be formed to have one or more durometers. Further, while two lumens are shown (e.g., 7024a and 7024b), additional lumens can be provided. For example, more than one lumen can be provided around the perimeter of the central lumen 7024a. Lumens can be provided at the 12, 3, 6, and 9 o'clock locations on the perimeter of central lumen 7024a. Other positions can also be used.

FIG. 55 is a partial perspective view of distal end portions of components of the of system 7000 with outer layer 7028 shown as transparent. As previously described, outer liner layer 7028 can be in the form of a reflowed material, but other materials, such as sleeves can be used. Distal tube portion 7018 includes flex windows and ports that can act as inlets to allow the reflow of material to bond or attach the outer layer 7028 to the tube.

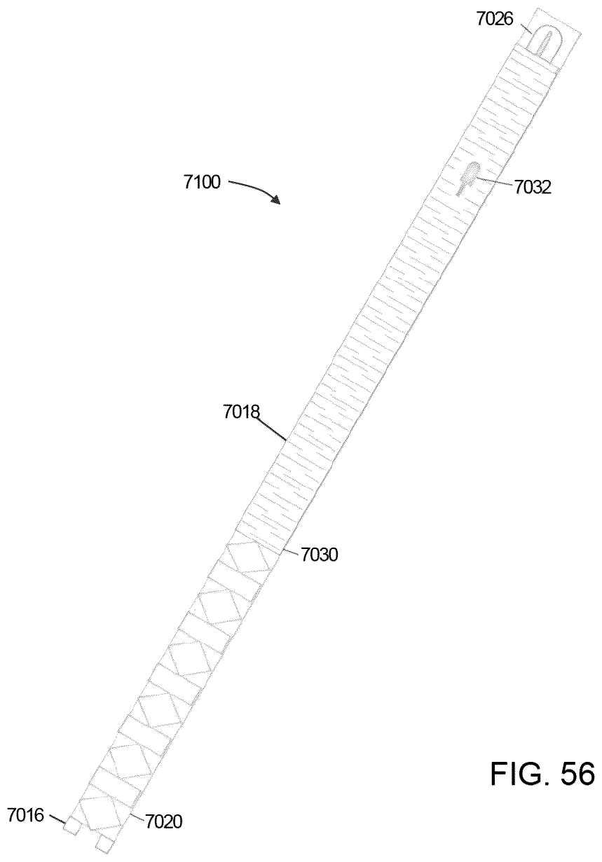

FIG. 56 illustrates a schematic view of one implementation of a patterned hypotube 7100. The hypotube includes patterned proximal tube portion 7020 and distal tube portion 7018. As previously described, one or more patterns can be formed or cut into the tube portions to provide, adjust or fine-tune the axial, flexural, and torque response/characteristic of each tube portion. The patterns can be laser-cut or formed by any other appropriate cutting or forming means. The tube can be made of nitinol, stainless steel, polymers, plastics, or any other suitable material. An opening 7032 can be provided in proximal tube portion 7020 for passage of one or more wires or control elements 7002. While one opening is shown, multiple openings can be provided in the tube portions for passage of other components including actuation/control elements, sensors, etc.

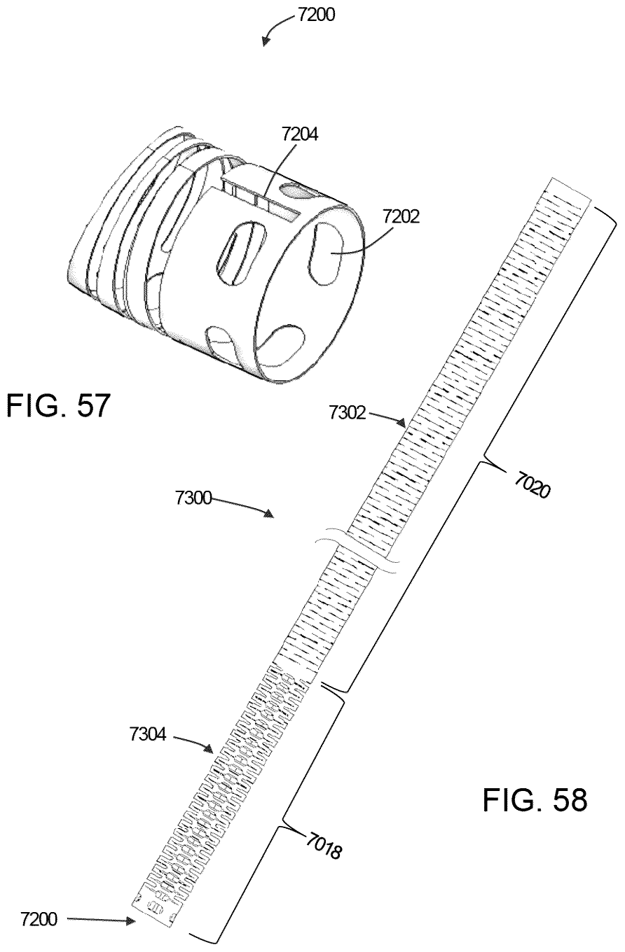

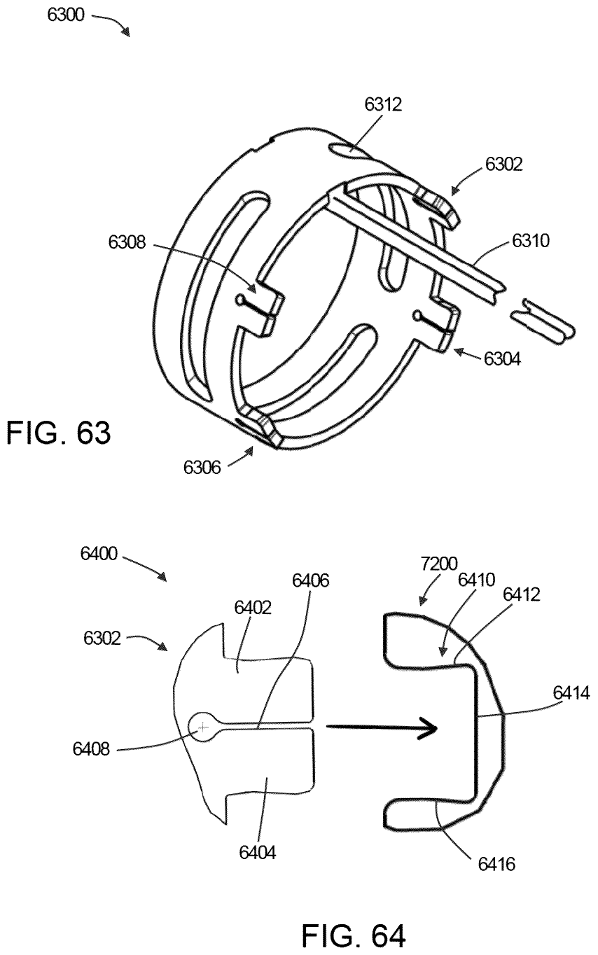

FIG. 57 illustrates one implementation of a patterned hypotube distal end in the form of distal end portion 7200. Distal end portion 7200 can be connected to a pull ring device or element (e.g., 7016) and includes a plurality of openings 7202. Openings 7202 can be used as ports for the reflow of outer layer 7028 material around the distal end portion 7200. Distal end portion 7200 also includes a slot 7204 for positioning and/or interfacing with a pull ring. Slot 7204 is arranged to mate with a projection of the pull ring or to provide clearance for one or more wires or actuation elements to connect to the pull ring (e.g., see FIG. 63). While one slot 7204 is shown, multiple slots can be provided at various locations on distal end portion 7200.

FIG. 58 illustrates another implementation of a patterned hypotube in the form of hypotube 7300. Hypotube 7300 includes a plurality of patterned portions, which include proximal tube portion 7020 and distal tube portion 7018. Proximal tube portion 7020 can be provided with a first pattern 7302. As previously described, first pattern 7302 can be, for example, a spiral cut pattern (including an interrupted spiral cut pattern). Other patterns can also be used. A second pattern 7304 can be provided on distal tube portion 7018. Second pattern 7304 can include one or more patterns of flex windows and ports cut into the wall distal tube portion 7018, which will be described in more detail below. Also, one or more transition portions can be provided in hypotube 7300 between, for example, proximal tube portion 7020 and distal tube portion 7018.

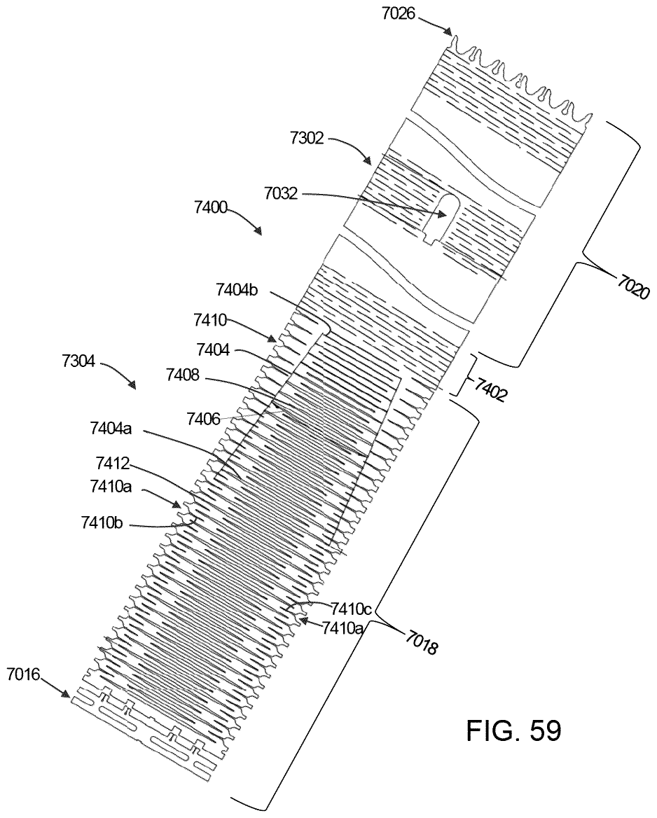

FIG. 59 illustrates another implementation of a patterned hypotube in the form of hypotube 7400. Hypotube 7400 is shown in a flat, unrolled state to illustrate the various cut patterns therein. All the patterns can be (laser) cut into a flat or round hypotube (e.g., either seam welded or seamless). In the case of a flat sheet of material, the material can be curled into its final tubular configuration with the longitudinal side end portions joined together by any suitable means such as welding, soldering, adhering, etc. Also, pull ring 7016 can be a separately cut component manufactured from the same material in the same or similar way to hypotube 7400 and subsequently connected thereto.

Still referring to FIG. 59, a first pattern 7302 is cut into proximal tube portion 7020. In one implementation, first pattern 7302 includes a spiral cut pattern, which can be an interrupted spiral cut pattern. The spiral cut pattern includes a plurality of cuts, windows and/or ports for the reflow of jacket, liner, and/or durometer material and for axial, flexural and torsional response and strength. In one implementation, the first pattern 7302 provides proximal tube portion 7020 with a less flexible response than distal tube portion 7018 to thereby provide pushability. A suitable interrupted spiral cut pattern can be defined by a pitch distance, kerf width, cut length or degree length, and uncut length or degree length. As previously described, each of these parameters can be varied to form one or more cut patterns, which impact the flexibility and strength of proximal tube portion 7020. In one example, pattern 7302 can have a pitch distance of 0.030 to 0.050, kerf width of 0.0015+/−0.0004 mm, a cut length of 0.216+/−0.04 mm, uncut length of approximately 0.0441+/−0.004 mm or any sub range of these ranges. Hypotube 7400 wall thickness can range from approximately 0.007 to 0.010 mm. Other values can be used based on the hypotube wall thickness and desired response/characteristics. Also as previously described, first pattern 7302 can include a plurality of patterns or sub-patterns having different characteristics.

Distal tube portion 7018 includes second pattern 7304, which in one implementation can include a plurality of cuts or openings for various flex window and port patterns. Example flex window cuts or patterns are shown at first flex windows 7408 and second flex windows 7412. In some implementation, the pattern 7304 alternates/repeats first flex window 7408 and second flex window. These can extend along either the entire or partial length of distal tube portion 7018 as shown in FIG. 59. In one implementation, the flex window cuts or openings have a width of 0.0166 mm+/−10% and have a spacing between window cuts or openings of 0.0166 mm+/−10%. Also, in one implementation, all the flex windows have the same length and/or width. In another implementation, as shown in FIG. 59, the flex windows 7408, 7412 can have different lengths (and/or widths). In FIG. 59, the flex window pattern includes a flex window pattern 7404 with flex windows 7412 that transition from a starting or first size or length 7404a to an ending or second size or length 7404b and the flex windows 7408 have a constant width. However, in some implementations, both flex windows 7408, 7412 can transition from a first length to a second length. In the implementation shown, the flex windows gradually or progressively transition or change size along the length or distance of flex window pattern 7404. This change in size or length of the flex window pattern 7404 provides additional control over the flexural response of distal tube portion 7018. In the implementation shown, the gradual or progressive reduction in flex window length progressively lessens the flexural response characteristic of the hypotube in that portion. Conversely, the gradual or progressive increase in flex window length progressively increases the flexural response characteristic of the hypotube in that portion. In one implementation, the flex window pattern 7404 can extend for approximately half the length of the distal tube portion 7018. In other implementations, it can extend for longer or shorter than that length. Flex window pattern 7404 can also act as a transition region or portion 7030 between two sections of the hypotube (i.e., a transition between two or more different cut patterns or two or more different flexural response/characteristics).

Thus, a plurality of flex window patterns can be provided in one or more portions of the hypotube with each flex pattern contributing to the flexural response characteristics of that portion of the tube. In the implementation shown, two flex window patterns are shown. A first or distal flex window pattern, which is constant in size, shape and spacing, and a second or more proximal flex window pattern, which is progressively different in flex window size for a portion of the hypotube. Other combinations are possible to include more or less than one pattern of flex windows (e.g., size, shape, spacing) to control the flexural response characteristics of the hypotube.

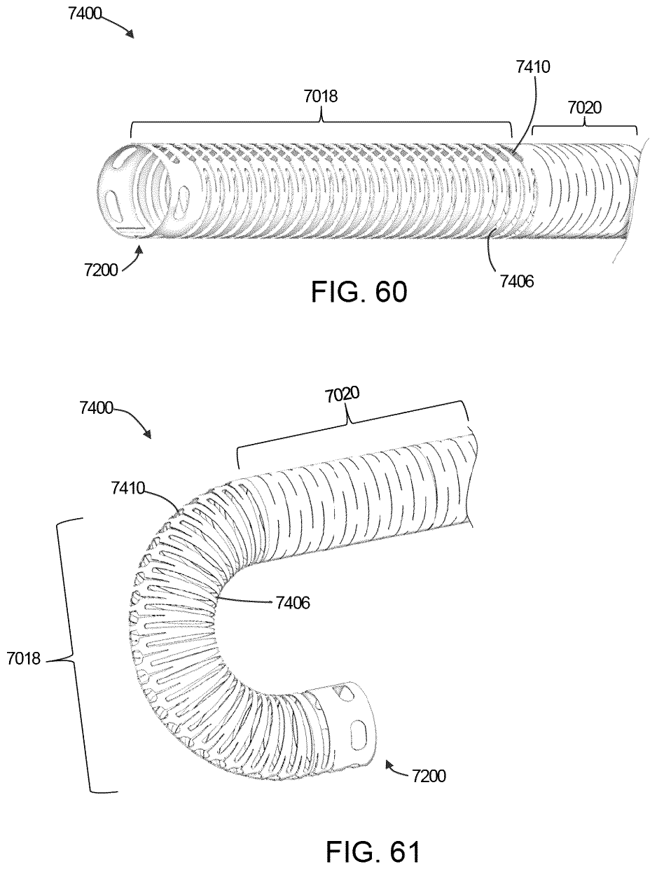

Distal tube portion 7018 can also include one or more reflow port patterns 7410. Reflow port pattern 7410 includes one or more cuts, windows and/or openings that allow for the reflow jacket, liner or durometer material (as previously described) to be included in the multi-lumen sheath system. In one implementation, reflow port pattern 7410 can extend along the entire length or select lengths of distal tube portion 7018. In the implementation shown in FIG. 59, which is a flat or sheet material cut with the various patterns, reflow port pattern 7410 is shown implemented along the longitudinal sides thereof. Thus, half the pattern is cut into one longitudinal side and the other half is cut into the other longitudinal side. As previously described, the flat sheet material is rolled to form a tube and thus joining the two halves of the reflow port pattern 7410 (e.g., see FIGS. 60 and 61). In other implementations, reflow port pattern 7410 need not be made along the longitudinal sides of the flat hypotube material, but can be made in whole or singly within the material (i.e., not split in halves). For example, the patterns can be cut directly in the tube.

In the implementation shown, reflow port pattern 7410 includes a central opening 7410a and optional side cuts 7410b and 7410c. The pattern can be uniform along the length of distal tube portion 7018 or can be different comprising a plurality of different patterns or sub-patterns. Also, in the implementation shown, reflow port pattern 7410 can be disposed on the hypotube at a location that is approximately opposite the flex windows. Some overlap can also be implemented as shown (e.g., see port side cuts 7410b and 7410c and flex windows 7408). In other implementations, the overlap can be omitted or varied. Reflow port pattern 7410, as well as the flex window patterns (e.g., 7404 and 7408) and spiral cut patterns (e.g., 7302), can be used as reflow ports for the introduction of reflow jacket, liner, spacer, etc. material (as previously described).

FIG. 60 is a partial perspective view of hypotube 7400 in an unbent or unflexed state or configuration. This view also illustrates hypotube 7400 after it has been curved or rolled from its initial flat state shown in FIG. 59 or the hypotube can be cut from a preformed tube. This view also illustrates an implementation of hypotube 7400 with a uniform flex window pattern 7406 along the length of distal tube portion 7018. As previously described, distal tube portion 7018 can have one or more flex window cut patterns. This view also illustrates reflow port pattern 7410 in its complete or finalized state (e.g., see FIG. 59 showing reflow port pattern 7410 implemented in halves along the longitudinal sides of the flat hypotube material prior to curving or rolling of the material to form a tube.)

FIG. 61 is a partial perspective view of hypotube 7400 with distal tube portion 7018 in a bent or flexed state or configuration. Distal tube portion 7018 can include a range of flex including from approximately 0 to 180 degrees. In some implementations, this flexure is with minimal impact on straightness (e.g., flexure) of the proximal tube portion 7020. The flex window patterns 7404 and/or 7406 allow distal tube portion 7018 to flex or bend (e.g., a radiused flex) to form substantially a “J” shape (e.g., 180 degrees bend). The flex window patterns 7404 and/or 7406 allow distal tube portion 7018 to have reduced compression forces during flexure due to their size, shape and arrangement on distal tube portion 7018. Flex window pattern 7404 (see FIG. 59) adds or provides for a better or more radiused flex and the reduction and/or prevention of inner tube diameter constriction (e.g., ridging) during flexure. This reduces the amount of insertion and retraction forces necessary within the sheath/catheter/lumen due to reduced ridging/constriction within the tube.

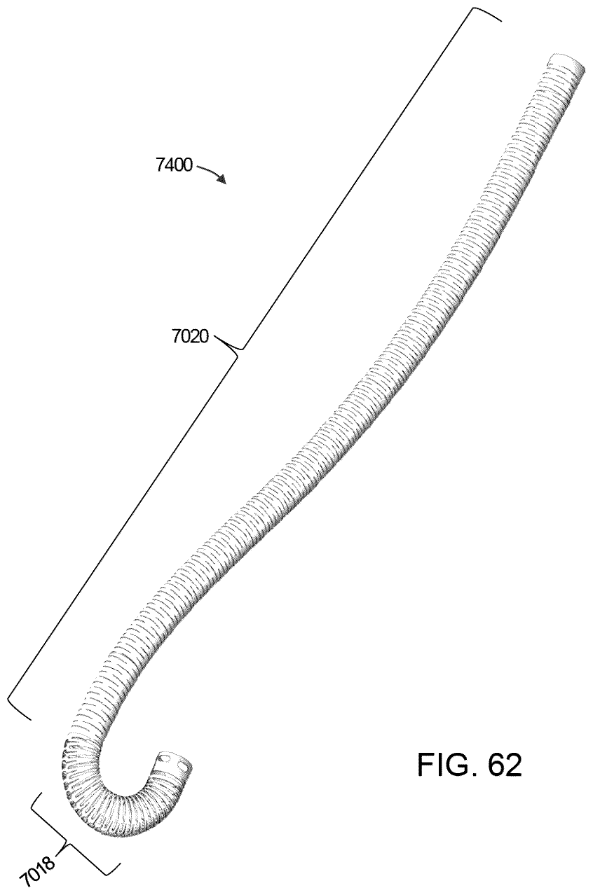

FIG. 62 is a partial perspective view of hypotube 7400 in second bent or flexed state or configuration. In this state, proximal tube portion 7020 is shown having some flexure or partial flexure, but not as much as distal tube portion 7018. Thus, each portion of the hypotube can have a flexure response/characteristic and the flexure response/characteristic can vary from portion to portion. In the embodiment shown, it can be desirable to allow proximal tube portion 7020 to have some flexure in order to facilitate passage through the patient's anatomy. As previously described, the amount of flexure can be controlled by the cut pattern in the proximal tube portion 7020, which can be an interrupted spiral cut pattern in one implementation or example.

FIG. 63 is a perspective view of one implementation of a pull ring element 6300. FIG. 63 shows pull ring element 6300 in its curved or rolled final state and FIG. 59 shows the pull ring element (shown as pull ring 7016) in its flat state. The pull ring 6300 can be cut from a preformed tube. Referring to FIG. 63, pull ring element 6300 includes a body having one or more connection crenulations, extensions, or projections 6302, 6304, 6306, and 6308. In one implementation, these are used to connect pull ring element 6300 to hypotube 7400. The connection can be made by various means including, for example, snap-fit, interference fit, suture, adhesive, welding, soldering, and/or other connecting arrangements. While four such connection projections have been shown, more or less than four can be used and their location on pull ring element 6300 can also be varied. Pull ring element 6300 can further include one or more cut patterns having windows or ports 6312 for allowing reflow of jacket, liner, and/or tip material, as previously described. One or more pull wires or actuation elements 6310 are connected to pull ring element 6300 in order to cause control (including flexure) of the distal tube portion 7018. For example, the actuation elements 6310 can be pulled to flex the distal portion as illustrated by FIG. 61 and can be relaxed to move and/or allow the distal portion to move to the straight condition illustrated by FIG. 60. The wire or actuation element connection can be made by any suitable means including suture, adhesive, welding, soldering, etc.

FIG. 64 is a magnified partial view of one implementation of an arrangement 6400 for connecting pull ring element 6300 to a hypotube. Arrangement 6400 includes a mating connection such as, for example, a snap-fit or interference fit connection. In this regard, one or more of the connection crenulations, extensions, or projections, such as 6302, can include first extension 6402, second extension 6404, gap 6406, and flex window 6408. Projections 6302, etc. are received in a mating recess 6410 of the hypotube. Mating recess 6410 includes side walls 6412 and 6416, and end wall 6414 arranged to matingly receive projections 6302, etc. Mating recess 6410 can be sized slightly smaller than projection 6302 to form an interference fit connection. So formed, extensions 6402 and 6404 would flex towards each other via the space provided by gap 6406 (which would reduce in size). Flex window 6408 reduces the amount of resistance to flexure of the extensions 6402 and 6404 as they are inserted to mating recess 6410. Beads, bumps, hooks, etc., and mating cuts or recesses can be provided in the arrangement to further facilitate the snap and/or interference fit connection. In other implementations, connection arrangements other than snap or interference fit can be used including, for example, sutures, welds, adhesives, soldering, etc. Furthermore, the exact size and shape of the mating components is not critical so long as they allow for connection of the pull ring element to the hypotube.

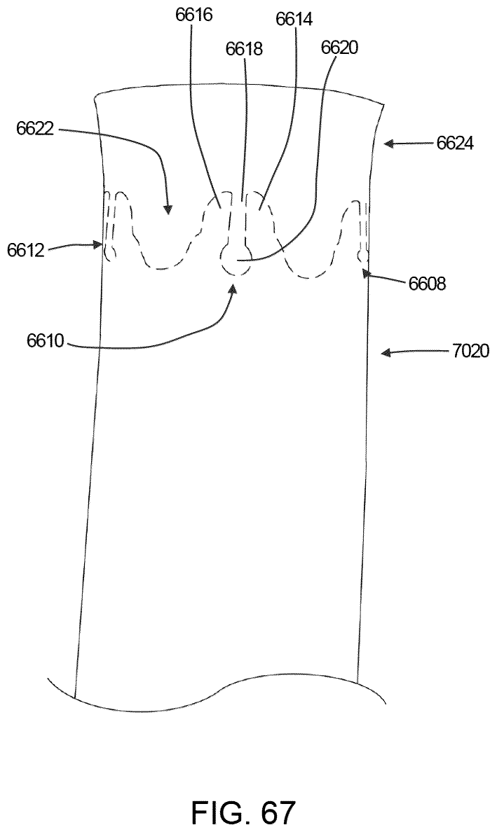

Referring now to FIGS. 66 and 67, various perspective views of one implementation of a proximal end portion 6600 of a hypotube, such as the hypotube described above, of a multi-layer sheath system is shown (see also FIG. 59 showing proximal end portion 6600 in its flat state as shown at 7026.) FIG. 66 shows proximal end portion 6600 of the tube and FIG. 67 shows proximal end portion 6600 of the hypotube with a portion of the outer jacket 7028 forming an interface 6624 for connection to a handle and/or control device. Proximal end portion 6600 can include in one implementation a plurality of extensions or fingers 6602, 6604, 6606, 6608, 6610, and 6612. The exact number of extensions/fingers is not critical and more or less than those shown can be used. In one implementation, each extension/finger 6602, etc., can have tabs 6614 and 6616, gap 6618, and flex opening 6620. A space 6622 can be provided between each extension/finger. The extensions/fingers 6602, etc. are arranged to allow the interface 6624 to be flared outward. The number of extensions or fingers can vary (including more or less than that shown). The size, length, and/or width of each extension or finger can also vary from that shown. Thinner extensions or fingers flex more easily than thicker or wider extensions or fingers. Also, the size of the openings 6620 can be larger or smaller with larger openings contributing to easy of flexing and smaller openings contributing to less ease of flexing. Hence, variations from implementations shown and described herein are contemplated.

Referring to FIG. 67, extensions/fingers 6602, etc., and their tabs 6614 and 6616 are arranged to spread or expand under insertion force or pressure at the interface 6624. In other implementations, a spreader device can be used to spread extensions/fingers 6602, etc., and their tabs 6614 and 6616 to the appropriate size for connection to a handle or other device. The exact size, shape and arrangement of extensions/fingers 6602 is not critical so long as they allow for the required connection of the sheath to the other catheter components of the system.

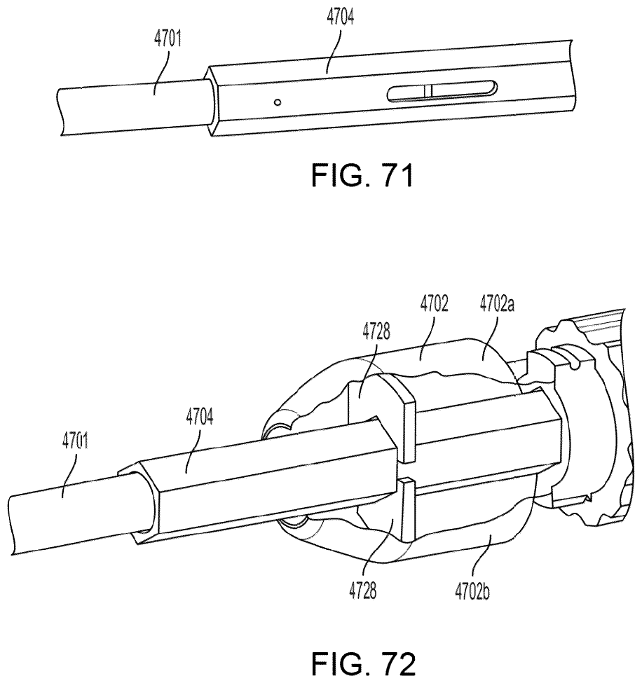

FIGS. 68-74 illustrate an example handle assembly 4700 that can be used with a steerable catheter 4701 (FIGS. 71-72). The steerable catheter 4701 can take any suitable form, such as, for example, the form of any steerable catheter described in the present application. The handle assembly 4700 can, however, be used with any other suitable steerable catheter, such as, for example, any steerable catheter disclosed herein.

In some implementations, the handle assembly 4700 can include a housing 4702, a guide member 4704 or rail, a follower 4706, a driver 4708, and an actuation member 4710. The housing 4702 can be made of any suitable material, such as, for example, plastic, fiberglass reinforced plastic, etc. In some implementations, as in the illustrated implementation, the housing 4702 includes a first housing portion 4702a and a second housing portion 4702b that are configured to connect to create the housing 4702 that houses at least a portion of the guide member 4704 or rail and the follower 4706. The first and second housing portions 4702a, 4702b can connect by any suitable means, such as, for example, a snap-fit connection, an adhesive connection, a welding connection, or any other suitable connection. In some implementations, the housing 4702 can be a single component or have more than two components that connect together.

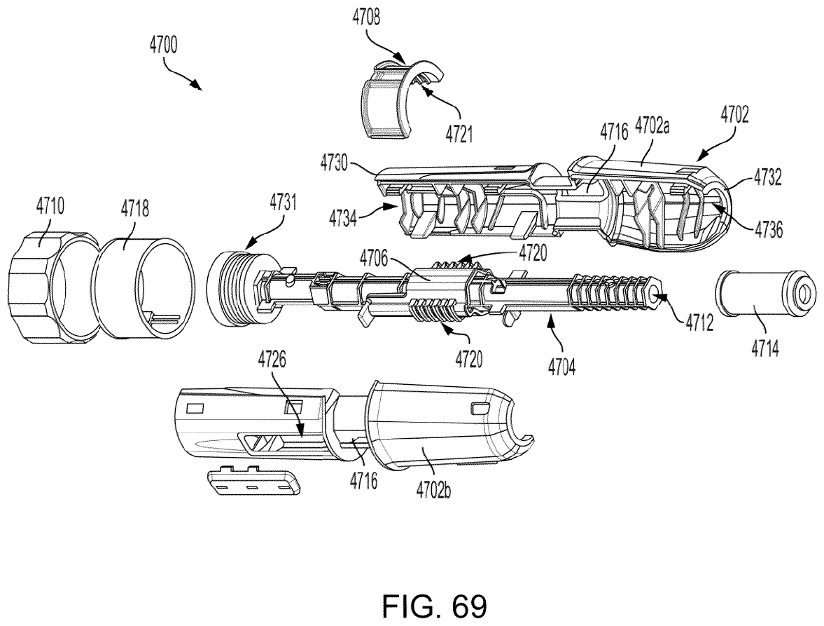

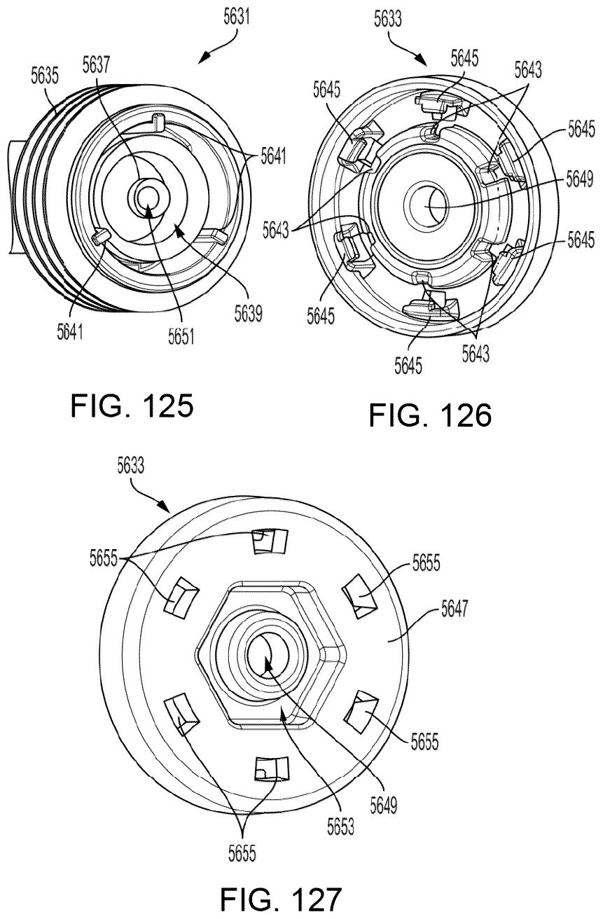

In some implementations, the handle assembly 4700 can include a seal assembly 4731 (FIGS. 69, 79, and 125-127) disposed at a proximal end of the guide member 4704 or rail. The seal assembly 4731 can take any suitable form, such as, for example, the form of the seal assembly 5631 shown in FIGS. 125-127, or any other suitable form for a seal assembly that is capable of working with a handle assembly of a steerable catheter assembly.

In some implementations, the guide member 4704 or rail includes a lumen 4712 and/or channel that is configured to receive a steerable catheter (e.g., any steerable catheter described in the present application or any other suitable steerable catheter). The follower 4706 is slidably attached to the guide member 4704 or rail such that the follower 4706 can move in both the proximal and distal directions relative to the guide member 4704 or rail.

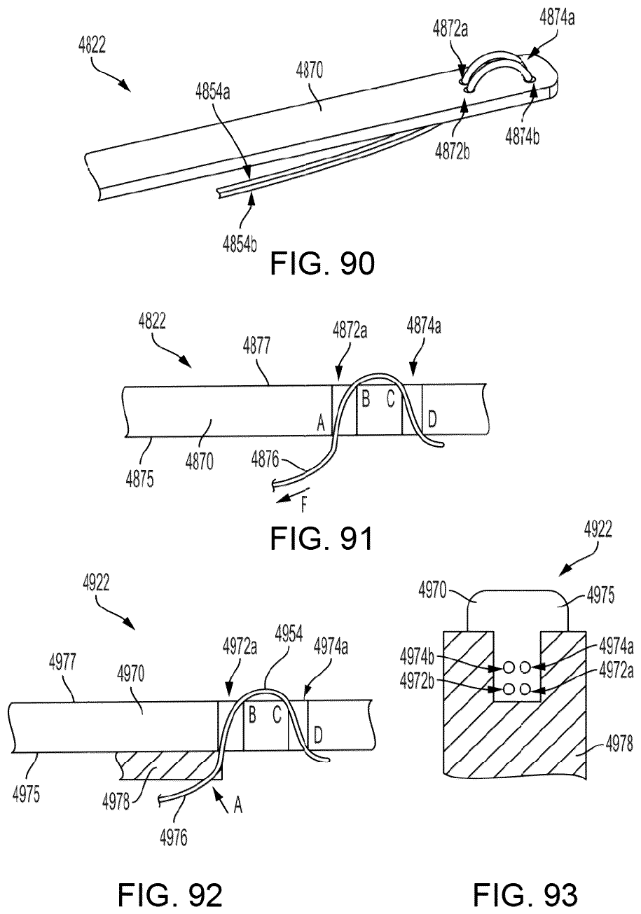

In some implementations, an actuation element (e.g., a wire, lie, shaft, tube, etc.) can be attached to the follower 4706 and the steerable catheter such that movement of the follower relative to guide member 4704 or rail causes the actuation element to engage and move the steerable catheter between various positions. In some implementations, the actuation element can be attached to the follower 4706 by a connector 4722 (FIGS. 79-81). The connector 4722 can take the form of any of the connectors shown in FIGS. 90-100 or any other suitable type of connector for attaching an actuation element to the follower 4706. A distal housing component 4714 can be positioned over and/or be coupled to a distal end of the guide member 4704 or rail.

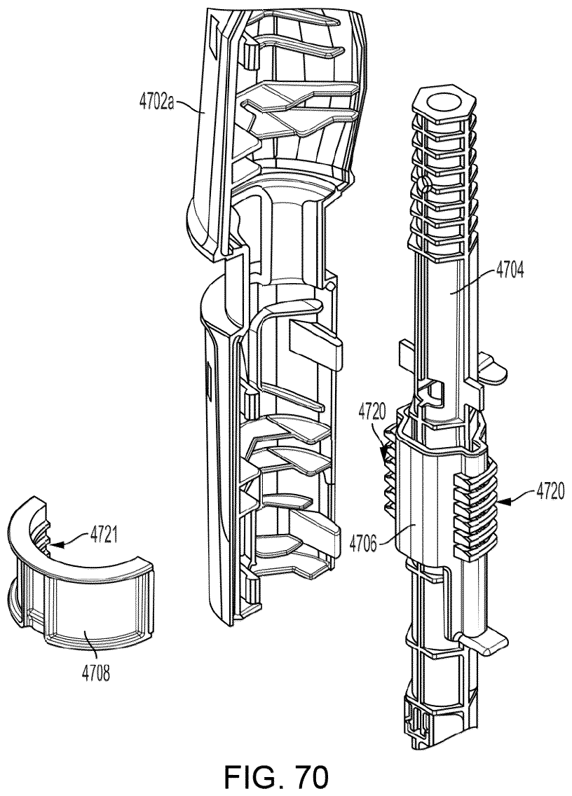

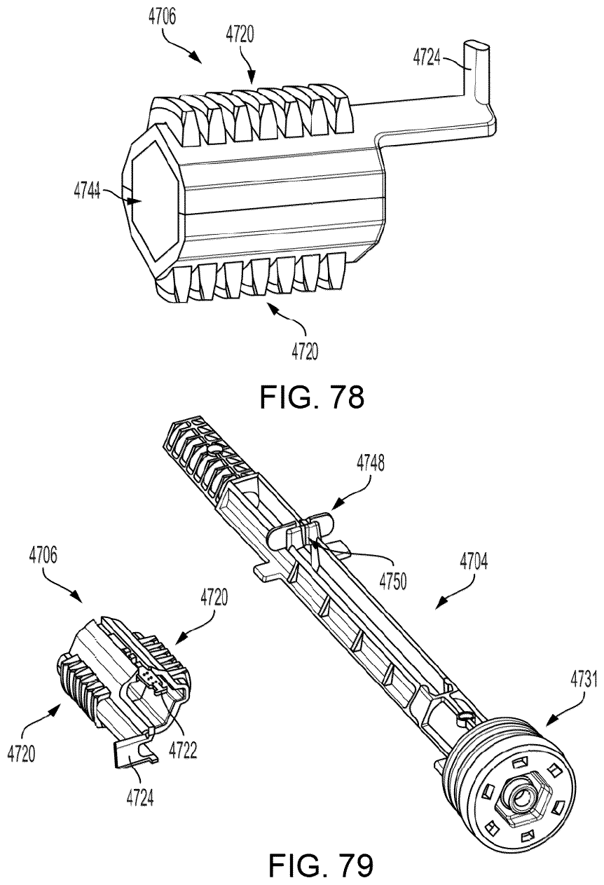

Referring to FIGS. 69 and 70, the follower 4706 can include external threads, and the driver 4708 can include internal threads 4721 for engaging the external threads 4720 (FIG. 78) of the follower 4706. In the illustrated implementation, the housing includes one or more windows 4716 that allow for the external threads of the follower 4706 to extend through the housing 4702 for engagement by the driver 4708. Rotation of the driver 4708 causes the follower 4706 to move relative to the driver 4708, which causes the follower 4706 to move relative to the guide member 4704 or rail. For example, rotation of the driver 4708 in a clockwise direction can cause the follower 4706 to move in a distal direction relative to the guide member 4704 or rail, and rotation of the driver 4708 in a counterclockwise direction can cause the follower 4706 to move in a proximal direction relative to the guide member 4704 or rail, or vice versa. In the illustrated example, only a half of the driver 4708 is illustrated, but it should be understood that the driver 4708 includes another half that is a mirror image thereof that connects to the illustrated portion to create the driver 4708. In some implementations, the driver 4708 can be a single component rather than a component that includes multiple portions that are connected together.

Referring to FIGS. 68-70 and 76-77, in some implementations, an optional actuation member 4710, such as the illustrated knob, or a lever, slider, etc. can be attached to the driver 4708 such that a user can engage the actuation member 4710 to rotate the driver 4708 and move the follower 4706 relative to the guide member 4704 or rail. In some implementations, the driver 4708 can be engaged directly by the user. An optional ring 4718 can be positioned between the driver 4708 and actuation member 4710. The actuation member 4710 can take any suitable form that is configured for allowing a user to engage the actuation member 4710 to rotate the driver 4708. While the illustrated example shows the driver 4708 and actuation member 4710 being separate components, it should be understood that the driver 4708 and actuation member 4710 can be a single component. For example, the actuation member 4710 can have internal threads that are integral to the actuation member 4710 for engaging the follower 4706, rather than having a separate driver.

Referring to FIGS. 71-72, the housing 4702 and guide member 4704 or rail are configured to prevent rotation of the guide member 4704 or rail relative to the housing 4702. In the illustrated example, the guide member 4704 or rail has a hexagonal shape, and the housing 4702 includes walls 4728 that extend from an interior of the housing 4702 and are shaped to receive the hexagonal shape of the guide member 4704 or rail such that the walls 4728 prevent rotation of the guide member 4704 or rail relative to the housing 4702. While the illustrated example shows the guide member 4704 or rail having a hexagonal shape and the walls 4728 having a corresponding shape, it should be understood that the guide member 4704 or rail and walls 4728 can have any other suitable shape that prevents rotation of the guide member 4704 or rail relative to the housing 4702. For example, the guide member 4704 or rail can have any other type of polygonal shape or any other shape that is capable of engaging with the housing to prevent rotation of the guide member 4704 or rail relative to the housing 4702.

In the illustrated example, both the first portion 4702a and the second portion 4702b of the housing 4702 include walls 4728. However, it should be understood that the walls 4728 can be disposed on one or more portions of the housing 4702 as long as the walls 4728 are capable of engaging the guide member 4704 or rail to prevent rotation of the guide member 4704 or rail relative to the housing 4702.

The housing 4702 can include any suitable number of walls 4728 positioned between a proximal end 4730 (FIG. 69) and a distal end 4732 (FIG. 69). For example, the housing 4702 can include one or more walls, two or more walls, three or more walls, four or more walls, five or more walls, six or more walls, seven or more walls, eight or more walls, nine or more walls, ten or more walls, etc. In some implementations, one or both of a proximal opening 4734 (FIG. 69) and a distal opening 4736 of the housing can be shaped to engage the guide member 4704 or rail and prevent rotation of the guide member 4704 or rail relative to the housing 4702. In some implementations, the housing may not include any walls 4728 extending between the proximal end 4730 and the distal end 4732, but the openings alone can be shaped to engage the guide member 4704 or rail and prevent rotation of the guide member 4704 or rail relative to the housing 4702.

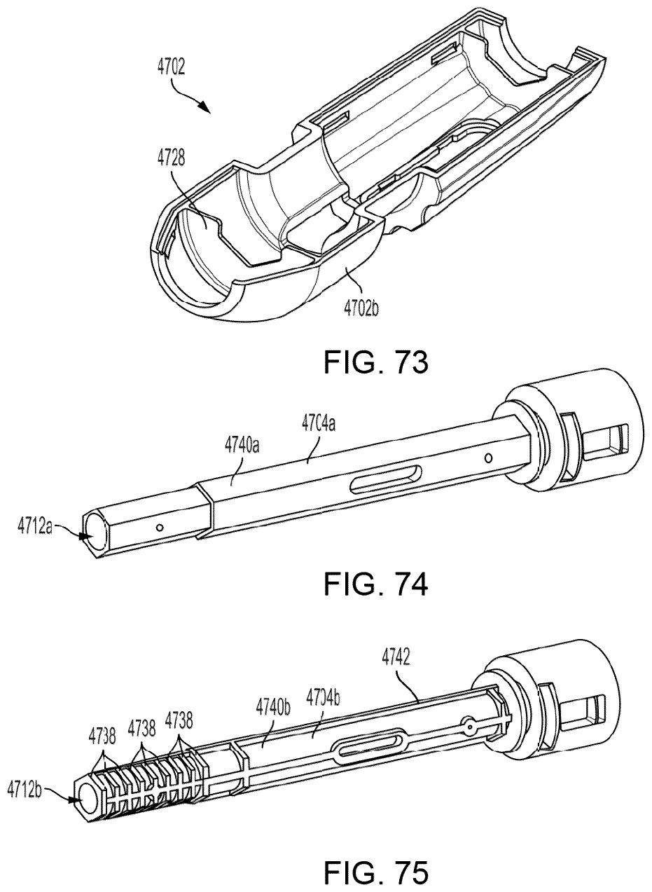

Referring to FIGS. 73-75, the housing 4702 can be configured to interact with guide members 4704a, 4704b or rails having different sized lumens 4712a, 4712b while still preventing the guide members 4704a, 4704b from rotating relative to the housing 4702. That is, the size of the lumen 4712a, 4712b depends on the size of the steerable catheter being attached to the guide member 4704a, 4704b, and the housing 4702 can be configured to engage with a guide member regardless of the size of the guide member 4704a, 4704b and prevent rotation of the guide member 4704a, 4704b relative to the housing 4702. FIG. 74 illustrates a guide member 4704a or rail having a lumen 4712a that is sized for a larger sized steerable catheter, and FIG. 75 illustrates a guide member 4704b or rail having a lumen 4712b that is sized for a smaller sized steerable catheter. In some implementations, the exterior perimeter 4740a of the guide member 4704a or rail can be larger than the exterior perimeter 4740b of the guide member 4704b or rail because of the larger sized lumen 4712a. The housing 4702 can be configured to engage with the guide member 4704a or rail that has a larger exterior perimeter and a guide member 4704b or rail that has a smaller exterior perimeter.

In some implementations, the housing 4702 can include one or more walls 4728 that extend from an interior surface of the housing 4702 for engagement with the guide members 4704a, 4704b. The walls 4728 can be shaped and sized for engagement with the larger guide member 4704a or rail, and the smaller guide member 4704b or rail can include one or more ribs 4738 that extend from its exterior perimeter 4740b that are configured to engage with the walls 4728 of the housing 4702. The distance that the ribs 4738 extend from the exterior perimeter 4740b of the smaller guide member 4704b or rail depends on the size of the exterior perimeter 4740b of the guide member 4704b or rail relative to the size of the exterior perimeter 4740a of the larger guide member 4704a or rail for which the housing 4702 is sized to accommodate. The use of ribs 4738 allows for any guide member having an exterior perimeter that is smaller than the larger guide member 4704a or rail to be used with the same housing 4702 as the larger guide member 4704b or rail.

In some implementations, in addition to or in replacement of the ribs 4738, the smaller guide member 4704a or rail can have one or more ribs 4742 that extend along a length of the guide member 4704b or rail, where the ribs 4742 are configured to engage the walls 4728 of the housing to prevent rotation of the guide member 4704b or rail relative to the housing 4702. That is, the ribs 4742 make the outer profile of the guide member 4704b the same size as the outer profile of the guide member 4704a. As such, the guide members 4704a, 4704b are sized such that the same walls 4728 or other same complimentary surfaces of the housing 4702a, 4702b engage and prevent rotation of both guide members 4704a, 4704b.

In some implementations, rather than having the ribs 4742, the guide members 4704a, 4704b are substantially the same, except the passages 4712a, 4712b are different to accept different sized catheters. The guide members 4704a, 4704b can be configured in any manner that allows the guide members 4704a, 4704b to accept differently sized catheters and be constrained and/or used with the same housing 4702a, 4702b.

FIGS. 78-79 illustrate an example follower 4706 that can be used with a guide member 4704 and/or rail of the handle assembly 4700. The follower 4706 is configured to be disposed on and move relative to the guide member 4704 or rail. The follower 4706 can include an optional indicator 4724 that is configured to extend through and/or be visible through an indicator window 4726 of the housing 4702.

In some implementations, a lumen 4744 of the follower 4706 can be shaped to correspond to the shape of the guide member 4704 or rail, and the shape of the lumen 4744 and the guide member 4704 or rail can be configured to prevent rotation of the follower 4706 relative to the guide member 4704 or rail. In the illustrated example, the lumen 4744 has a hexagonal shape that corresponds to the hexagonal shape of the guide member 4704 or rail. However, it should be understood that the lumen 4744 can have any other suitable shape that allows the follower 4706 to move along the length of the guide member or rail while also preventing rotation of the follower 4706 relative to the guide member 4704 or rail. For example, the lumen 4744 can have any other type of polygonal shape or any other suitable shape the prevents the follower 4706 from rotating relative to the guide member 4704 or rail.

Referring to FIG. 79, the follower 4706 can be configured to attach to a connector 4722 that attaches the actuation element to the steerable catheter such that movement of the follower 4706 along the guide member 4704 or rail causes the connector 4722 to move and the connected actuation element to engage the steerable catheter to move the steerable catheter between various positions. The connector 4722 can attach to the follower 4706 by any suitable means, such as, for example, being slidably disposed in a slot in the follower, one or more fasteners, adhesive, welding, friction fit, etc. The connector 4722 can take any suitable form, such as, for example, any form for a connector that connects an actuation element to the follower 4706 described in the present application.

Referring to FIG. 79, the guide member 4704 or rail can include a guide member 4748 for the actuation element. The guide member 4748 is configured to engage the actuation element such that the actuation element maintains a desired position between the follower 4706 and the steerable catheter. In the illustrated implementation, the guide member 4748 includes a slot 4750 for receiving an actuation element that includes a wire, where the slot 4750 maintains the wire along a desired route between the follower 4706 and the steerable catheter.

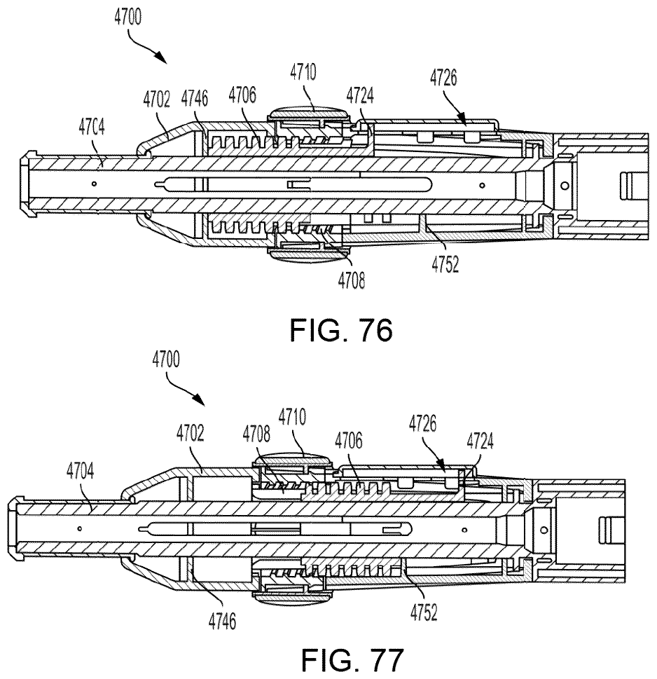

Referring to FIG. 76, when the follower 4706 is positioned at a distal position relative to the housing 4702, the indicator 4724 of the follower 4706 is positioned at a distal end of the window 4726. Referring to FIG. 77, when the follower 4706 is positioned at a proximal position relative to the housing 4702, the indicator 4724 of the follower 4706 is positioned at a proximal end of the window 4726. The positioning of the indicator 4724 within the window 4726 allows a user to determine the position of the follower 4706 relative to the guide member 4704 or rail and, consequently, the position and/or degree of flex of the steerable catheter because of the connection between the follower 4706 and the steerable catheter via an actuation element and connector 4722.

Referring to FIGS. 76 and 77, the housing 4702 can have optional distal stop member(s) 4746 and/or optional proximal stop member(s) 4752 that prevent or inhibit movement of the follower 4706 relative to the housing 4702 beyond desired boundaries. In addition to, or in replace of, the stop members 4746, 4752 of the housing 4702, the guide member 4704 or rail can include stop members that restrict movement of the follower 4706 relative the housing 4702 and guide member 4704 or rail.

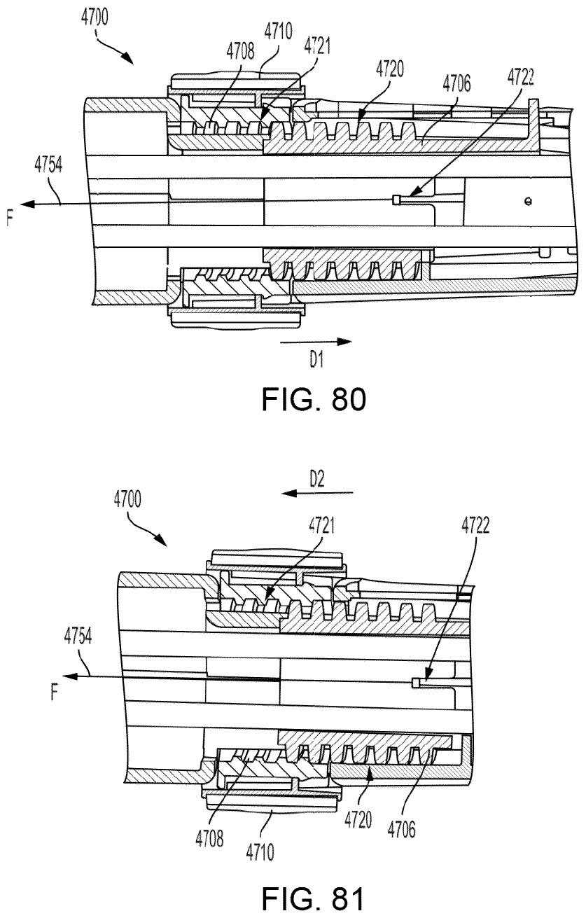

Referring to FIGS. 80-81, in some implementations, the exterior threads 4720 of the follower 4706 and the interior threads 4721 of the driver 4708 are configured (e.g., the longitudinal lengths of the exterior threads 4720 and the interior threads 4721 are selected) to limit the flex of the catheter and provide a clutch. That is, the coupling between the exterior threads 4720 and the interior threads 4721 ends when the catheter reaches a predetermined maximum flex. Further rotation of the driver 4708 does not further flex the catheter. Rather, the driver 4708 rotates without moving the follower 4706.

In some implementations, the handle assembly 4700 can optionally be configured to prevent disengagement between the exterior threads 4720 of the follower 4706 and the interior threads 4721 of the driver 4708 as the follower 4706 is being moved to a proximal position relative to the housing 4702. For example, the connection between the follower 4706 and a steerable catheter via an actuation element 4754 and the connector 4722 (e.g., the connector 4722 shown in FIG. 79) can create a force F on the follower 4706 that causes the threads 4720 of the follower 4706 to maintain contact with the threads 4721 of the driver 4708 (i.e., a proximal end of the threads 4721 stays in contact with a distal end of the threads 4720). Referring to FIG. 80, movement of the follower 4706 in the proximal direction D1 can cause the threads 4720 of the follower 4706 to move beyond the threads 4721 of the driver 4708. When this happens, the force F caused by the actuation element 4754 on the follower 4706 maintains the engagement between the follower 4706 and driver 4708.

Referring to FIG. 81, maintaining engagement between the threads 4720 of the follower 4706 and the threads 4721 of the driver 4708 allow a user to engage the actuation member 4710 to rotate the driver 4708 and move the follower 4706 in a distal direction D2 relative to the housing 4702, even after the driver 4708 has been rotated further than the point at which the catheter is fully flexed. In some implementations, a biasing member, such as a spring can be coupled to (e.g., press against) the follower 4706 to supplement the force F applied by the actuation element 4754.

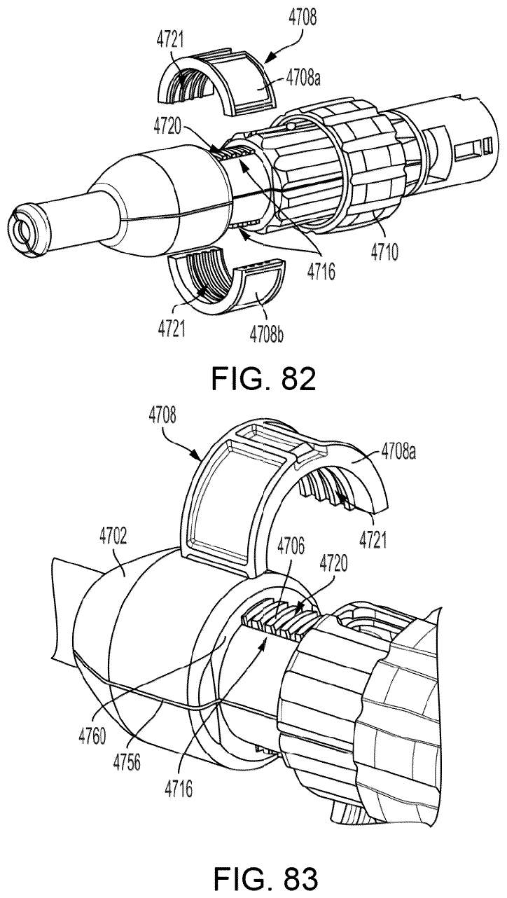

FIGS. 82-85 illustrate an example engagement between the actuation member 4710 and the follower 4706, as well as an example portion of the housing 4702 that allows for smooth rotation of the actuation member 4710 relative to the housing 4702. Referring to FIGS. 82-83, threads 4720 of the follower 4706 are shown extending through the window 4716 of the housing 4702. The driver 4708 is shown as having a first driver portion 4708a and a second driver portion 4708b that includes threads 4721 that are connected such that the threads 4721 of each portion 4708a, 4708b are in engagement with the threads 4720 of the follower 4706. The first and second portions 4708a, 4708b can connect by any suitable means, such as, for example, a snap-fit connection, an adhesive connection, a welding connection, etc., or any other suitable connection. In some implementations, the driver 4708 can be a single component that includes threads 4721 for engaging the threads of the follower 4706.

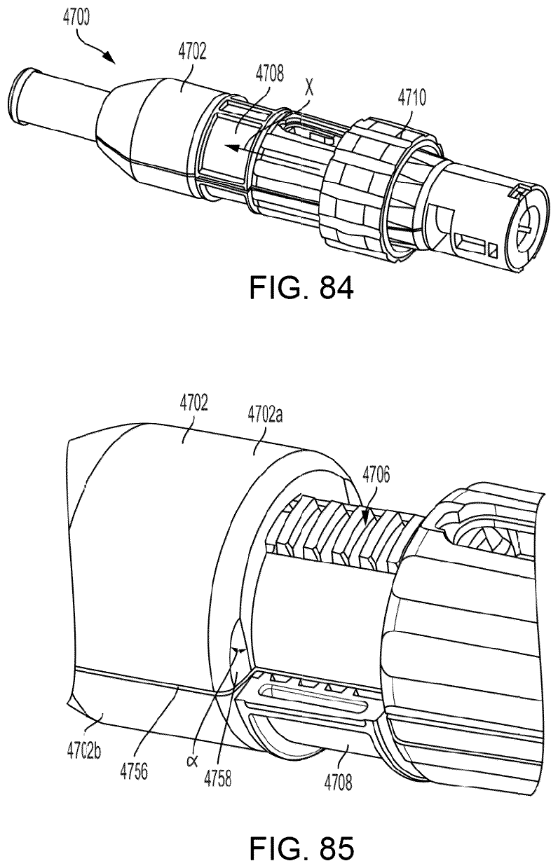

Referring to FIG. 84, after the driver 4708 is attached to the housing 4702 and engaged with the follower 4706, the actuation member 4710 can be attached to the driver 4708 such that the actuation member 4710 can be engaged by a user to rotate the driver 4708. In the illustrated example, the actuation member 4710 is shown being slid over the housing 4702 in a direction X to connect the actuation member 4710 to the driver 4708. The actuation member 4710 can be attached to the driver 4708 by any suitable means, such as, for example, a snap-fit connection, an adhesive connection, a welding connection, etc.

Referring to FIGS. 83 and 85, in the illustrated example, the housing 4702 has a first housing portion 4702a and a second housing portion 4702b that are connected to create the housing 4702. An interface 4756 between the two housing portions 4702a, 4702b can create edges that can allow for the actuation member 4710 to be caught on during rotation of the actuation member 4710 relative to the housing 4702. In some implementations, the housing 4702 can include one or more angled portions 4758 at a position where the actuation member 4710 and interface 4756 meet to prevent the actuation member 4710 from catching on the edges of the housing portions 4702a, 4702b. For example, the angled portions 4758 can include an angle α such that the angled portions 4758 extend away from a wall 4760 where the actuation member 4710 engages the housing 4702. The angle α can be between about 1 degree and about 20 degrees.

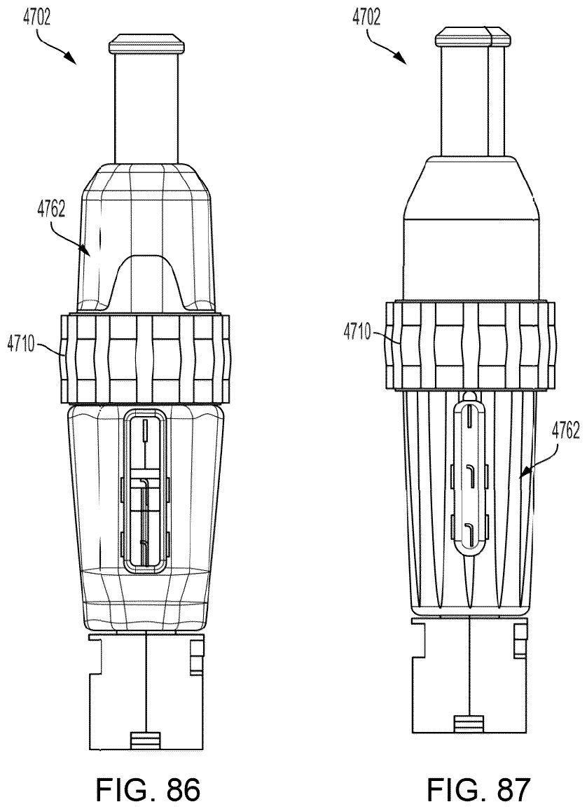

Referring to FIGS. 86-89, the housing 4702 of the handle assembly 4700 can be configured such that the exterior 4762 of the housing 4702 can have various appearances, but the internal features and/or components of the housing can remain unchanged. For example, the handle assemblies can be used to replace existing handles for a variety of applications where the handles being replaced, and other handles of the application have different looks and feels. That is, handles for implanting a treatment device, a valve repair device, a replacement valve, a docking station, and/or handles made by different manufacturers can look different from one another. By configuring the housing 4702 of the handle assembly 4700 such that the exterior 4762 of the housing 4702 can have various appearances with the internal features and/or components of the handles being the same, handles for different applications and/or from different manufacturers having different shapes, looks, and/or feels can be replaced by the handles disclosed by the present application without substantially changing the mechanical design and/or operation of the handle.

Referring to FIGS. 90-91, an example connector 4822 is shown that can be used with a handle assembly of a steerable catheter, such as, for example, the handle assembly 4700 shown in FIGS. 68-89. The connector 4822 can be configured to attach to a follower (e.g., follower 4706 of the handle assembly 4700 described in the present application) such that actuation element(s) 4854a, 4854b (e.g., wire(s), line(s), shaft(s), tube(s), rod(s), etc.) can attach to the follower such that movement of the follower causes the actuation element to engage the steerable catheter and flex the steerable catheter between various positions.

In some implementations, the connector 4822 can have a body 4870, one or more distal openings 4872a, 4872b extending through the body 4870, and one or more proximal openings 4874a, 4874b extending through the body 4870. The body 4870 can be made from a variety of different materials, such as metals, such as, for example, steel, aluminum, nitinol, etc., and/or or non-metals, such as plastic, etc.

Each actuation element 4854a, 4854b can be connected to the body 4870 of the connector 4822 by extending through both a corresponding distal opening 4872a, 4872b and a corresponding proximal opening 4874a, 4874b. In some implementations, as in the illustrated example, two actuation elements 4854a, 4854b are shown being attached to the connector 4822, with the first actuation element 4854a being extended through first distal and proximal openings 4872a, 4872b, and with the second actuation element 4854b being extended through second distal and proximal openings 4872b, 4874b. It should be understood, however, the connector 4822 can have any suitable number of distal and proximal openings for connecting any suitable number of actuation elements to the connector 4822. Referring to FIG. 90, In some implementations, as in the illustrated example, the actuation elements 4854a, 4854b are shown in a parallel configuration as the actuation elements move through the corresponding distal and proximal openings. In some implementations, the actuation elements 4854a, 4854b can be in a crossed configuration. For example, the actuation element 4854a can extend through the distal opening 4872a and the proximal opening 4874b, and the actuation element 4854b can extend through the distal opening 4872b and the proximal opening 4874a.

Referring to FIG. 91, the first actuation element 4854a is shown being attached to the body 4870 of the connector 4822. While the connection between the first actuation element 4854a and the connector 4822 is being described with reference to FIG. 91, it should be understood that the second actuation element 4854b can be attached to the connector 4822 in a similar manner. In some implementations, a portion 4876 of the actuation element extends from the steerable catheter (not shown), through the distal opening 4872a from a bottom surface 4875 to a top surface 4877 of the body 4870, and through the proximal opening 4874a from the top surface 4877 to the bottom surface 4875 to create a stitch connection of the actuation element 4854a to the connector 4822. In some implementations, the actuation element 4854a can be attached to the connector 4822 by a stitch connection that includes the actuation element 4854a extending through the distal opening 4872a from the top surface 4877 to the bottom surface 4875 and through the proximal opening 4874a from the bottom surface 4875 to the top surface 4877. Still referring to FIG. 91, the stitch connection between actuation element 4854a and the connector 4822 creates points of contact (A, B, C, D) between the actuation element 4854a and the connector 4822. In some implementations, the stitch connection allows for the forces on the actuation element from the connection with the connector 4822 to be distributed across these points of contact (A, B, C, D) to prevent slippage and/or breakage of the actuation element as a Force F is provided to the actuation element.

Referring to FIGS. 92-93, another example connector 4922 is shown that can be used with a handle assembly of a steerable catheter, such as, for example, the handle assembly 4700 shown in FIGS. 68-89. In some implementations, the connector 4922 can be configured to attach to a follower (e.g., follower 4706 of the handle assembly 4700 described in the present application) such that one or more actuation elements 4954 (e.g., wire(s), line(s), shaft(s), tube(s), rod(s), etc.) can attach to the follower such that movement of the follower causes the actuation element 4954 to engage the steerable catheter and flex the steerable catheter between various positions.

In some implementations, the connector 4922 can have a body 4970, one or more distal openings 4972a, 4972b extending through the body 4970, one or more proximal openings 4974a, 4974b extending through the body 4970, and a support member 4978 attached to a bottom surface 4975 of the body 4970. The body 4970 can be made of a metal material, and/or a non-metal material. The support member 4978 can be made of a plastic material. In other examples, the support member 4978 can be made of any other suitable non-plastic material. The support member 4978 can be attached to the main body 4970 by a sliding connection, welding, an adhesive connection, and/or friction fit, or any other suitable connection.

In some implementations, the actuation element(s) 4954 can be connected to the body 4970 of the connector 4922 by extending through both a corresponding distal opening 4972a, 4972b and a corresponding proximal opening 4974a, 4974b. In the illustrated example, the connector includes two proximal openings 4972a, 4972b and two distal openings 4974a, 4974b for connecting two actuation elements to the connector 4922. It should be understood, however, the connector 4922 can have any suitable number of distal and proximal openings for connecting any suitable number of actuation elements to the connector 4922. When more than one actuation element 4954 are attached to the connector 4922, the actuation elements can be attached in a parallel configuration or a crossed configuration.

Referring to FIG. 92, the actuation element 4954 is shown being attached to the body 4970 of the connector 4922. While the connection between the first actuation element 4954a and the connector 4922 is being described with reference to FIG. 92, it should be understood that one or more additional actuation elements can be attached to the connector 4822 in a similar manner. In some implementations, a portion 4976 of the actuation element 4954 extends from the steerable catheter (not shown), through the distal opening 4972a from a bottom surface 4975 to a top surface 4977 of the body 4970, and through the proximal opening 4974a from the top surface 4977 to the bottom surface 4975 to create a stitch connection of the actuation element 4954 to the connector 4922.

Still referring to FIG. 92, the support member 4978 is attached to the body 4970 such that the support member 4978 is proximate the distal opening 4972a on the bottom surface 4975 of the body 4970, which allows for the most distal contact point (at contact point A) between the actuation element 4954 and the connector 4922 to be at the support member 4978 instead of the body 4970. In the illustrated example, the stitch connection between actuation element 4954 and the connector 4922 can create points of contact (A, B, C, D) between the actuation element 4854a and the connector 4822. In some implementations, the material of the support member 4978 (relative to the material of the body 4970) is configured to provide relief at contact point A to the actuation element to prevent breakage of the actuation element as a force F is provided to the actuation element. The stitch connection also allows for the forces on the actuation element 4954 from the connection with the connector 4922 to be distributed across the other three points of contact (B, C, D) to further prevent breakage of the actuation element as the Force F is provided to the actuation element.

In other examples, the actuation element 4854a can be attached to the connector 4822 by a stitch connection that includes the actuation element 4854a extending through the distal opening 4872a from the top surface 4877 to the bottom surface 4875 and through the proximal opening 4874a from the bottom surface 4875 to the top surface 4877. In these examples, the support member 4978 can be attached to the top surface 4977 of the body 4970 such that the support member 4978 is proximate the distal opening 4972a on the top surface 4977 of the body 4970, which allows for the most distal contact point between the actuation element 4954 and the connector 4922 to be at the support member 4978 instead of the body 4970.

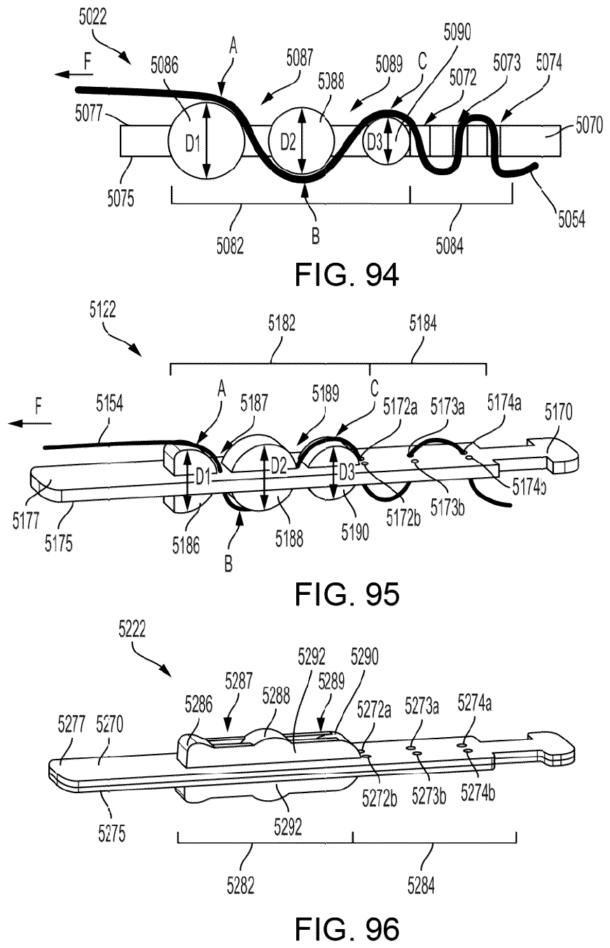

Referring to FIG. 94, another example connector 5022 is shown that can be used with a handle assembly of a steerable catheter, such as, for example, the handle assembly 4700 shown in FIGS. 68-89. In some implementations, the connector 5022 can be configured to attach to a follower (e.g., follower 4706 of the handle assembly 4700 described in the present application) such that one or more actuation elements 5054 attach to the follower such that movement of the follower causes the actuation element 5054 to flex the steerable catheter and move the steerable catheter between various positions.

In some implementations, the connector 5022 can have a body 5070 that includes a strain relief portion 5082 and a connection portion 5084. The body 5070 can be made of a metal material and/or a non-metal material, such as, for example, plastic. In some implementations, the strain relief portion 5082 is configured to reduce the stresses provided to the actuation element 5054 when a force F is provided to the actuation element 5054, and the connection portion 5084 is configured to attach the actuation element 5054 to the body 5070.

In some implementations, as in the illustrated example, the strain relief portion 5082 includes a first strain relief member 5086, a second strain relief member 5088, and a third strain relief member 5090. A first strain relief opening 5087 can be positioned between the first and second strain relief members 5086, 5088, and a second strain relief opening 5089 can be positioned between the second and third strain relief members 5088, 5090. In some implementations, the strain relief members 5086, 5088, 5090 are configured to be engaged by the actuation element 5054 as the actuation element 5054 extends between the steerable catheter (not shown) and the connection portion 5084 of the connector 5022.

In some implementations, the strain relief members 5086, 5088, 5090 can have a rounded or curved portions at the locations A, B, C where the actuation element 5054 engages the strain relief members. The curved portions reduce the amount of strain provided to the actuation element 5054 as a force F is provided to the actuation element 5054. In the illustrated example, each of the strain relief members 5086, 5088, 5090 has a circular shape. In some implementations, the most distally positioned strain relief member (i.e., the strain relief member 5086 shown in FIG. 94) can a diameter that is greater than or equal to the size of the other strain relief members. In the illustrated example, the diameter D1 of the first strain relief member 5086 is larger than the diameter D2 of the second strain relief member 5088, and the diameter D2 of the second strain relief member 5088 is larger than the diameter of the third strain relief member 5090. The strain relief members 5086, 5088, 5090 can have any other suitable shape having a curved portion for engagement with the actuation element 5054, such as, for example, an oval shape, a semicircular shape, a semi-curved shape, or any other suitable shape that includes a curved portion.

In some implementations, the actuation element 5054 can be connected to the body 5070 of the connector 5022 by a stitch connection by extending the actuation element 5054 through three corresponding openings 5072, 5073, 5074 of the connection portion 5084 of the connector 5022. The illustrated example shows a single actuation element 5054 extending through three corresponding openings 5072, 5073, 5074. However, it should be understood that the connector can have additional groupings of openings for receiving additional actuation elements.

In some implementations, the actuation element 5054 can extend from the strain relief portion 5082 and through the opening 5072 from the top surface 5077 of the body 5070 to the bottom surface 5075 of the body 5070. In some implementations, the actuation element 5054 can then extend through the opening 5073 from the bottom surface 5075 to the top surface 5077 and can extend through the opening 5074 from the top surface 5077 to the bottom surface 5075. In some implementations, the actuation element 5054 can contact various portions of the body 5070 as it extends through the openings 5072, 5073, 5074 such that a strain is placed on the actuation element 5054, but the amount of strain is reduced due to the engagement between the actuation element and the strain relief portion 5082. The engagement of the actuation element 5054 with the strain relief portion 5082 is configured to prevent breakage of the actuation element 5054 as a force F is provided to the actuation element due to the distribution of forces across the strain relief members 5086, 5088, 5090.

Referring to FIG. 95, another example connector 5122 is shown that can be used with a handle assembly of a steerable catheter, such as, for example, the handle assembly 4700 shown in FIGS. 68-89. In some implementations, the connector 5122 can be configured to attach to a follower (e.g., follower 4706 of the handle assembly 4700 described in the present application) such that one or more actuation elements 5054 can attach the follower to a steerable catheter such that movement of the follower causes the actuation element 5154 to engage the steerable catheter and move the steerable catheter between various positions.

In some implementations, the connector 5122 can have a body 5170 that includes a strain relief portion 5182 and a connection portion 5184. The body 5170 can be made of a metal material and/or a non-metal material, such as, for example, plastic. The strain relief portion 5182 is configured to reduce the stresses provided to the actuation element 5154 when a force F is provided to the actuation element 5154, and the connection portion 5184 is configured to attach the actuation element 5154 to the body 5170.

In some implementations, as shown in the illustrated example, the strain relief portion 5182 includes a first strain relief member 5186, a second strain relief member 5188, and a third strain relief member 5190. A first strain relief opening 5187 can be positioned between the first and second strain relief members 5186, 5188, and a second strain relief opening 5189 can be positioned between the second and third strain relief members 5188, 5190. The strain relief members 5186, 5188, 5190 are configured to be engaged by the actuation element 5154 as the actuation element 5154 extends between the steerable catheter (not shown) and the connection portion 5184 of the connector 5122.

The strain relief members 5186, 5188, 5190 can have a rounded or curved portions at the locations A, B, C where the actuation element 5154 engages the strain relief members. The curved portions reduce the amount of strain provided to the actuation element 5154 as a force F is provided to the actuation element 5154. In some implementations, as shown in the illustrated example, the first strain relief member 5086 has a semi-circular shape, and the second and third strain relief members 5188, 5190 each have a circular shape. In some implementations, the most distally positioned strain relief member (i.e., the strain relief member 5186 shown in FIG. 95) can have a diameter that is greater than or equal to the size of the other strain relief members. In some implementations, as shown in the illustrated example, the diameter D1 of the first strain relief member 5186 and the diameter D2 of the second strain relief member 5188 are each larger than the diameter D3 of the third strain relief member 5190. The strain relief members 5186, 5188, 5190 can have any other suitable shape having a curved portion for engagement with the actuation element 5154, such as, for example, an oval shape, a semicircular shape, a semi-curved shape, or any other suitable shape that includes a curved portion.

In some implementations, the actuation element 5154 can be connected to the body 5170 of the connector 5122 by a stitch connection by extending the actuation element 5154 through three corresponding openings 5172a, 5173a, 5174a of the connection portion 5184 of the connector 5122. The illustrated example shows a single actuation element 5154 extending through three corresponding openings 5172a, 5173a, 5174a. However, it should be understood that the connector can have additional groupings of openings (e.g., openings 5172b, 5173b, 5174b) for receiving additional actuation elements. If more than one actuation element is used, the actuation elements can have a parallel configuration or a crossed configuration as the actuation elements extend through the strain relief portion 5182.

In some implementations, the actuation element 5154 can extend from the strain relief portion 5182 and through the opening 5172a from the top surface 5177 of the body 5170 to the bottom surface 5175 of the body 5170. In some implementations, the actuation element 5154 can then extend through the opening 5173a from the bottom surface 5175 to the top surface 5177 and can extend through the opening 5174a from the top surface 5177 to the bottom surface 5175.

In some implementations, the actuation element 5154 can contact various portions of the body 5170 as it extends through the openings 5172a, 5173a, 5174a such that a strain is placed on the actuation element 5154, but the amount of strain is reduced due to the engagement between the actuation element and the strain relief portion 5182. In some implementations, the engagement of the actuation element 5154 with the strain relief portion 5082 is configured to prevent breakage of the actuation element 5154 as a force F is provided to the actuation element due to the distribution of forces across the strain relief members 5186, 5188, 5190.

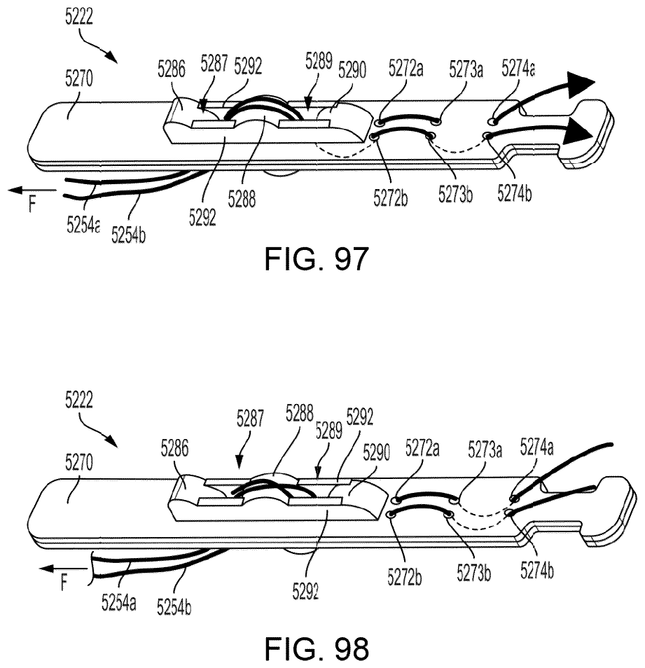

Referring to FIGS. 96-98, another example connector 5222 is shown that can be used with a handle assembly of a steerable catheter, such as, for example, the handle assembly 4700 shown in FIGS. 68-89. In some implementations, the connector 5222 can be configured to attach to a follower (e.g., follower 4706 of the handle assembly 4700 described in the present application) such that one or more actuation elements 5054 attach to the follower such that movement of the follower causes the actuation element(s) 5254a, 5254b (FIGS. 97-98) to engage the steerable catheter and move the steerable catheter between various positions.

In some implementations, the connector 5222 can have a body 5270 that includes a strain relief portion 5282 and a connection portion 5284. The body 5270 can optionally have a first half 5275 and a second half 5277 that can be a mirror image of the first half. The body 5270 can be made of a metal material or a non-metal material, such as, for example, plastic. The strain relief portion 5282 is configured to reduce the stresses provided to the actuation element 5254a, 5254b when a force F (FIGS. 97-98) is provided to the actuation elements 5254a, 5254b, and the connection portion 5284 is configured to attach the actuation elements 5254a, 5254b to the body 5270.

In some implementations, as in the illustrated example, the strain relief portion 5282 includes a first strain relief member 5286, a second strain relief member 5288, and a third strain relief member 5290. A first strain relief opening 5287 can be positioned between the first and second strain relief members 5286, 5288, and a second strain relief opening 5289 can be positioned between the second and third strain relief members 5288, 5290. The strain relief members 5286, 5288, 5290 are configured to be engaged by the actuation element 5254a as the actuation element 5254a extends between the steerable catheter (not shown) and the connection portion 5284 of the connector 5222.

The strain relief members 5286, 5288, 5290 can be configured to engage the actuation elements 5254a, 5254b in any suitable manner, such as, for example, a similar manner to the engagement between the actuation element 5154 and the strain relief members 5186, 5188, 5190 shown in FIG. 95, or by any other suitable manner. The strain relief members 5286, 5288, 5290 can take any suitable form, such as, for example, the form of the strain relief members 5186, 5188, 5190 shown in FIG. 95, or any other suitable form.

In some implementations, the actuation elements 5254a, 5254b can be connected to the body 5270 of the connector 5222 by a stitch connection. For example, referring to FIGS. 97-98, the actuation element 5254a can be connected to the body 5270 by extending the actuation element 5254a through three corresponding openings 5272a, 5273a, 5274a, and the actuation element 5254b can be connected to the body 5270 by extending the actuation element 5254b through three corresponding openings 5272b, 5273b, 5274b. The illustrated example shows corresponding openings for two actuation elements 5254a, 5254b being connected to the connector 5222. However, it should be understood that the connector can have additional groupings of openings for receiving additional actuation elements.

The actuation elements 5254a, 5254b can extend from the strain relief portion 5282 and through the corresponding openings of the connection portion 5284 by any suitable manner, such as, for example, the same manner described for the 5154 and the connector 5122 shown in FIG. 95. In some implementations, as in the illustrated example, the strain relief portion 5282 includes alignment walls 5292 that are configured to maintain the actuation elements 5254a, 5254b within the strain relief portion 5282. As the actuation elements 5254a, 5254b extend through the strain relief portion, the actuation elements 5254a, 5254b can have a parallel configuration (as shown in FIG. 97) or a crossed configuration (as shown in FIG. 98).

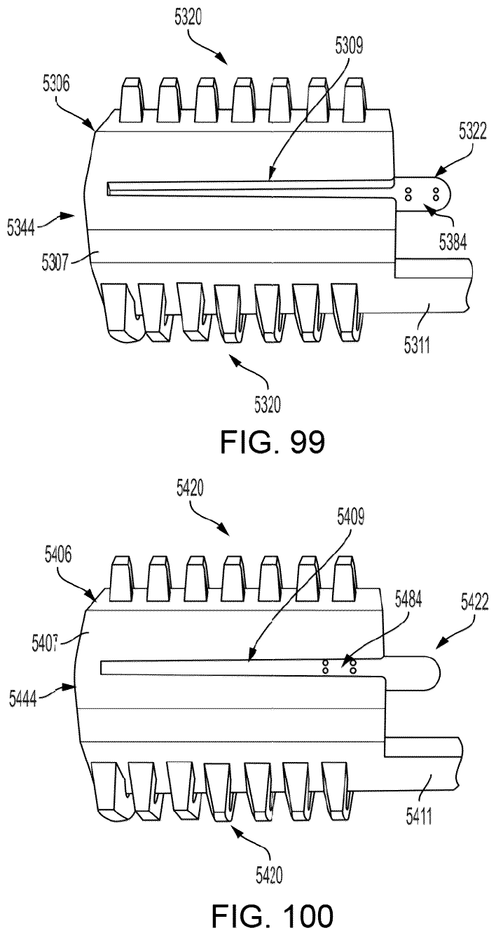

FIG. 99 illustrates an example connection between a follower 5306 and a connector 5322 for a handle assembly used with a steerable catheter (e.g., the handle assembly 4700 shown in FIGS. 68-89). The follower 5306 can take any suitable form, such as, for example, the form of the follower 4706 described with reference to FIGS. 68-89. The connector 5322 can take any suitable form, such as, for example, the form of any connector described in the present application for attaching a follower to a steerable catheter via an actuation element.

In the illustrated example, the follower 5306 has a body 5307 that defines a lumen 5344 and an optional opening 5309 that extends along a length of the body 5307. In some implementations, the follower 5306 can include external threads 5320 for being engaged by a driver (e.g., any driver for a handle assembly described in the present application) of the handle assembly. In some implementations, an extension member 5311 can extend from the body 5307 such that an indicator (e.g., similar to the indicator 4724 of the follower 4706 described in the present application) can extend from the extension member 5311 and be visible by a user during use of the handle assembly.

In some implementations, the connector 5322 can include a connection portion 5384 for attaching to an actuation element. The connection portion 5384 can be configured to receive the actuation element such that the actuation element is attached to the connector 5322 by a stitched connection. For example, the connector 5322 can include any of the features of the connectors shown in FIGS. 75-83 of the present application that allow for an actuation element to be attached to the connector 5322 by a stitched connection. In some implementations, the connection portion 5384 can include any other suitable type of connection features that allow for an actuation element to be attached to the connector 5322.

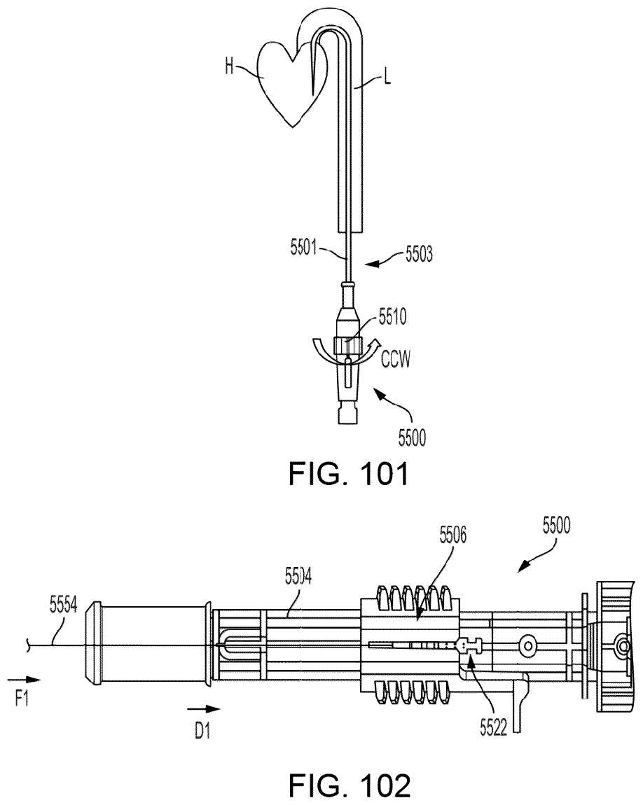

The connector 5322 can be connected to the follower 5306 by any suitable means, such as, for example, any means described in the present application for connection between the connector 4722 and the follower 4706 shown in FIGS. 68-85, or any means described in the present application for the connection between the connector 5522 and follower 5506 shown in FIGS. 101-104 and 125. The connector 5322 can be attached to the follower 5306 such that the connection portion 5384 of the connector 5322 is external to the lumen 5344 of the follower 5306. This positioning of the connector 5322 relative to the follower 5306 can allow for flexing of the connector 5322 when a force is applied to an actuation element that is connected to the connection portion 5384 of the connector 5322.

FIG. 100 illustrates an example connection between a follower 5406 and a connector 5422 for a handle assembly used with a steerable catheter (e.g., the handle assembly 4700 shown in FIGS. 68-89). The follower 5406 can take any suitable form, such as, for example, the form of the follower 4706 described with reference to FIGS. 68-89. The connector 5422 can take any suitable form, such as, for example, the form of any connector described in the present application for attaching a follower to an actuation element.

In some implementations, as in the illustrated example, the follower 5406 has a body 5407 that defines a lumen 5444 and an opening 5409 that extends along a length of the body 5407. In some implementations, the follower 5406 can include external threads 5420 for being engaged by a driver (e.g., any driver for a handle assembly described in the present application) of the handle assembly. In some implementations, an extension member 5411 can extend from the body 5407 such that an indicator (e.g., similar to the indicator 4724 of the follower 4706 described in the present application) can extend from the extension member 5411 and be visible by a user during use of the handle assembly.

In some implementations, the connector 5422 can include a connection portion 5484 for attaching to an actuation element. The connection portion 5484 can be configured to receive the actuation element such that the actuation element is attached to the connector 5422 by a stitched connection. For example, the connector 5422 can include any of the features of the connectors shown in FIGS. 75-83 of the present application that allow for an actuation element to be attached to the connector 5422 by a stitched connection. In some implementations, the connection portion 5484 can include any other suitable type of connection features that allow for an actuation element to be attached to the connector 5422.

In some implementations, the connector 5422 can be connected to the follower 5406 by any suitable means, such as, for example, any means described in the present application for connection between the connector 4722 and the follower 4706 shown in FIGS. 68-85, or any means described in the present application for the connection between the connector 5522 and follower 5506 shown in FIGS. 101-104 and 125. In some implementations, the connector 5422 can be attached to the follower 5406 such that the connection portion 5484 of the connector 5422 is disposed within the lumen 5444 of the follower 5406 and proximate the opening 5409. This positioning of the connector 5422 relative to the follower 5406 allows the actuation element to extend through the opening 5409 to connect to the connection portion 5484. This positioning of the connector 5422 relative to the follower 5406 provides additional support to the connector 5422 by the follower 5406, which can reduce the amount of stress provided to the actuation element as a force is provided to the actuation element.

FIGS. 101-104 and 125 illustrate an example steerable catheter assembly 5503 for engaging a heart H of a patient. The steerable catheter assembly 5503 includes a steerable catheter 5501 and a handle assembly 5500 for manipulating the steerable catheter 5501. The steerable catheter 5501 can take any suitable form, such as, for example, any form for a steerable catheter described in the present application. The handle assembly 5500 can take any suitable form, such as, for example, any form for a handle assembly described in the present application.

In some implementations, the handle assembly 5500 can include an actuation member 5510, a driver (not shown), a guide member or rail 5504, and a follower 5506, where these components can take any suitable form described in the present application, such as the form described with reference to FIGS. 68-89. In some implementations, the handle assembly 5500 can be configured such that a user can engage the actuation member 5510 to rotate the driver such that the driver engages the follower 5506 to move the follower 5506 relative to the guide member or rail 5504.

In some implementations, a connector 5522 can be attached to the follower 5506, and an actuation element 5554 can be attached to both the steerable catheter 5501 and the follower 5506 such that movement of the follower 5506 relative to the guide member or rail 5504 causes the steerable catheter 5501 to move between flexed configuration(s) and unflexed configuration(s). The connector 5522 can take any suitable form, such as, for example, any form of the connectors described in the present application with reference to FIGS. 90-98.

Referring to FIGS. 101 and 102, rotation of the actuation member 5510 in the counterclockwise direction CCW causes the follower 5506 to move in a proximal direction D1 relative to the guide member or rail 5504, which creates a force F1 on the actuation element 5554 that causes the steerable catheter 5501 to flex within the lumen L of the patient such that a distal end of the steerable catheter 5501 can move through the lumen L into the heart H of the patient. While the illustrated example shows the counterclockwise CCW movement of the actuation member 5510 moving the steerable catheter 5501 to the flexed position, it should be understood that the handle assembly 5500 can be configured such that a clockwise movement of the actuation member 5510 moves the steerable catheter 5501 to the flexed position.

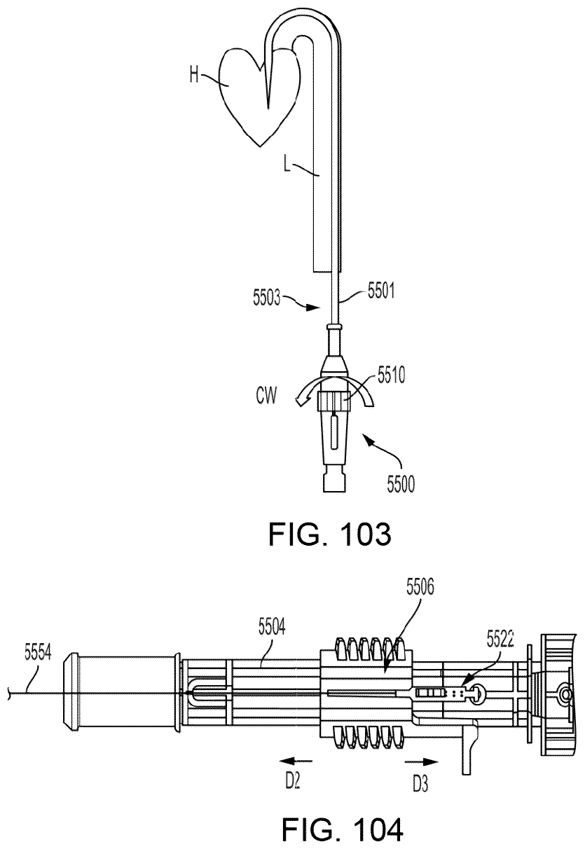

Referring to FIGS. 103 and 104, rotation of the actuation member in the clockwise direction CW causes the follower 5506 to move in a distal direction D2 relative to the guide member 5504. This movement of the follower 5506 typically causes the force F1 (FIG. 102) to be reduced, which allows the steerable catheter 5501 to move from the flexed configuration to an unflexed configuration. However, in some instances (e.g., as shown in FIG. 103), the lumen L of the patient may have a shape that causes the steerable catheter 5501 to maintain a flexed configuration. When this occurs, the actuation element 5554 can be compressed due to the movement of the follower 5506 in the distal direction D2.

In some implementations, as illustrated, the connector 5522 can be slidably attached to the follower 5506 such that the connector 5522 moves in a proximal direction D3 relative to the follower 5506 (e.g., the follower moves distally in the handle, while the connector remains in place) when the lumen L of the patient causes the steerable catheter 5501 to remain in the flexed position. For example, the connector 5522 can be slidably attached to the follower 5506 by being installed in a slot of the follower that has clearance around the outer surface of the connector. When the steerable catheter 5501 is moved to a portion of the lumen L that does not cause the steerable catheter to be in the flexed position, the connector 5522 can be configured to move in the distal direction D2 such that the connector 5522 moves back to its normal position relative to the follower 5506 (e.g., the position of the connector 5522 relative to the follower 5506 shown in FIG. 102). This slidable connection of the connector 5522 relative to the follower 5506 or inhibits application of a compressive force to the actuation element created by movement of the follower 5506 in the distal direction D2.

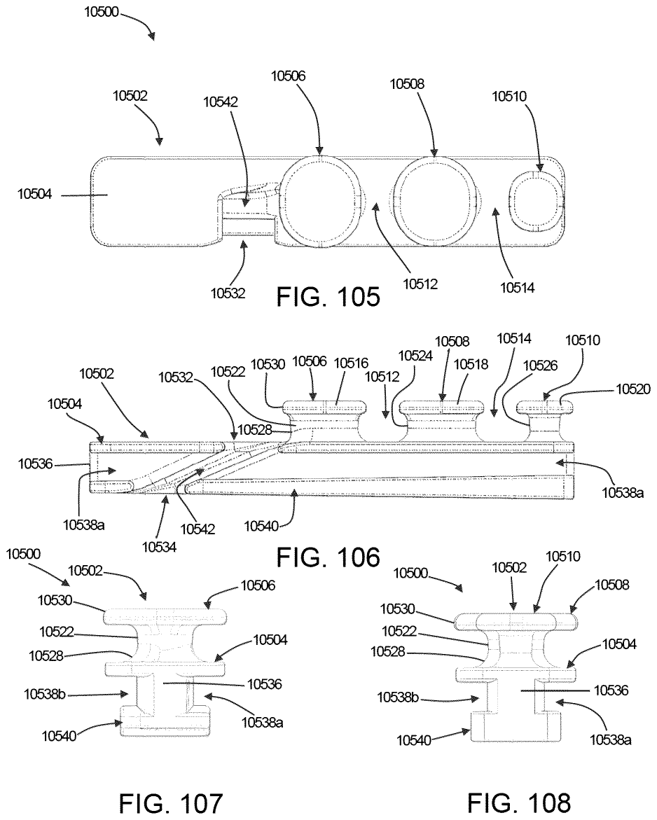

FIGS. 105-124 illustrate examples of connector devices and associated wire or actuation element connection/winding patterns. The illustrated connector devices can reduce the likelihood of, for example, broken or kinked wire/actuation elements, crossed wires, incorrect winding patterns, etc. The illustrated connector devices also simplify connection and winding by providing, for example, a post or cleat-based arrangement. Each post or cleat can have, for example, a head portion and a slimmer neck portion. This arrangement allows the actuation element to be wound around the slimmer neck portion. The larger head portion provides, for example, a guiding and/or retaining structure for proper placement of the actuation element to be wound around the post or cleat. Furthermore, slack can be reduced or minimized (e.g., as between multi-wire/actuation elements) by the open post arrangement(s) (e.g., size, shape, and/or dimensions). Thus, various post or cleat arrangements are disclosed along with various winding patterns.

Referring now to FIGS. 105-108, one implementation of a connector 10500 is provided. Connector 10500 includes a body having, for example, a base portion 10504 and one or more projections, posts or cleats 10506, 10508, 10510 arranged thereon. One or more gaps or spaces 10512 and 10514 are also provided between the posts or cleats. In the implementation shown, the posts or cleats do not need to have the same configuration. For example, post or cleat 10510 is arranged to be smaller than the other posts or cleats. In other implementations, the posts or cleats can have the same configuration. Body 10502 also includes openings 10532 and 10534, along with a passageway 10542 for one or more wire or actuation elements to be wound around the posts or cleats.

Body 10502 further includes support portion 10536 having, for example, flange portion 10540 and channel recesses 10538a and 10538b. This arrangement is used to mount connector 10500 to, for example, a follower device such as follower 5506. Channel recesses 10538a and 10538b can have a tapered or narrowing shape in to implement, for example, an interference fit or connection with the follower device. In other implementations, a non-tapered or uniform channel shape can also be used.

In the implementation shown, posts or cleats 10506, 10508, and 10510 have head portions 10516, 10518, and 10520, and neck portions 10522, 10524, and 10526, respectively. The neck portions are shown as being smaller or slimmer than the head portions. The neck portions can include curved or rounded side wall surfaces such as shown at 10528. The head portions can also include curved or rounded side wall surfaces such as shown at 10530. As will be described in more detail, one or more wire or actuation elements are wound around the neck portions of the posts or cleats in order to connect the wire or actuation elements to the connector 10500. The larger head portions provide placement guidance during winding and retention both during and after winding of the one or more wires or actuation elements to the posts or cleats.

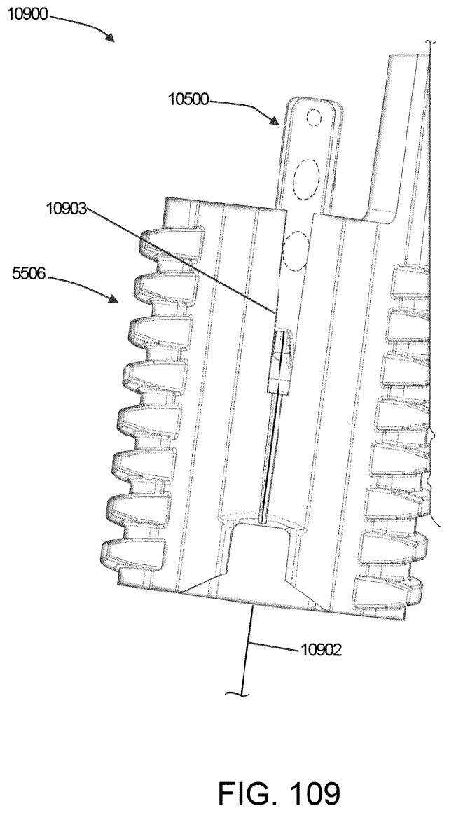

FIG. 109 illustrates one implementation 10900 of connector 10500 mounted to follower 5506. As previously described, connector 10500 can have a mounting channel arrangement (see 10538a and 10538b in FIGS. 105-108) for mounting connector 10500 to a follower device. The follower device can include a mounting slot 10903 for receiving the connector 10500 therein via the mounting channel arrangement of the connector 10500. Other mounting arrangements can also be used.

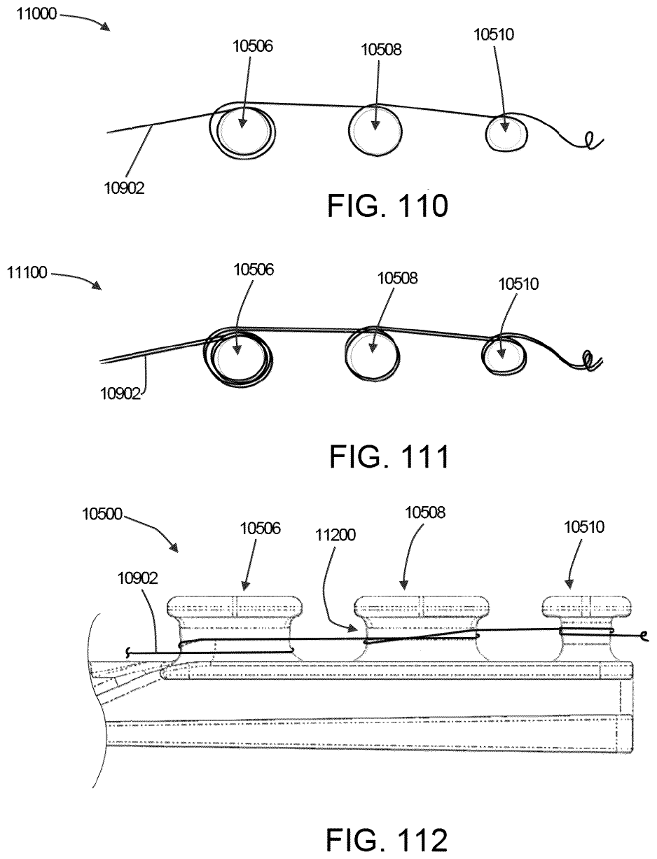

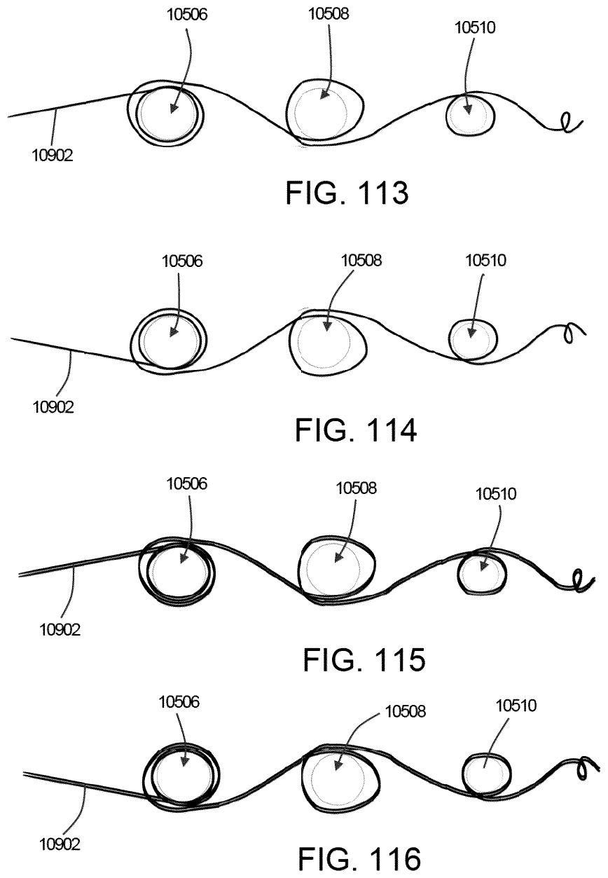

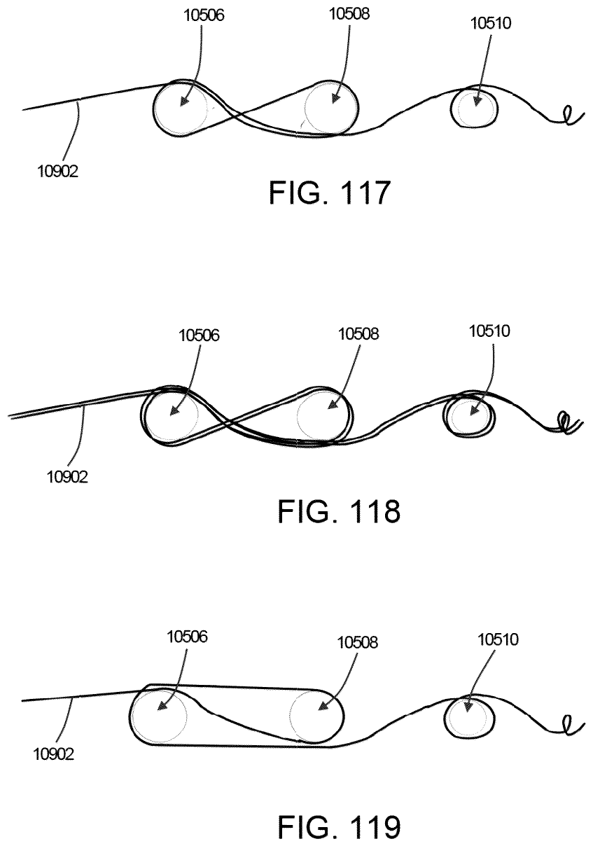

FIGS. 110-119 illustrate various winding or wrapping implementations for connecting one or more wires or actuation elements to, for example, connector 10500. In the various implementations, the winding or wrapping is accomplished by winding, wrapping or looping the actuation element(s) around one or more posts or cleats. FIG. 110 illustrates a first winding pattern 11000 using clockwise loops. This implementation includes a single wire or actuation element 10902 having two loops around post or cleat 10506 and single loops around posts or cleats 10508 and 10510. FIG. 111 illustrates a second winding pattern 11100 similar to first winding pattern 11000, but using more than one wire or actuation element 10902. FIG. 112 illustrates a third winding pattern having counterclockwise loops. This implementation includes single loops around all the posts and at least one post having an optional cross-over loop arrangement 11200. FIG. 113 illustrates a fourth winding pattern similar to the first winding pattern 11000 except that a counterclockwise loop is used for middle post or cleat 10508. FIG. 114 illustrates a fifth winding pattern similar to that shown in FIG. 113, except the winding directions have been reversed from that shown in FIG. 113. FIGS. 115 and 116 are similar to FIGS. 113 and 114, respectively, except that more than one wire or actuation element 10902 are used. FIGS. 117 and 118 illustrate another winding pattern using a figure “8” looping arrangement around posts or cleats 10506 and 10508. FIG. 119 illustrates another winding pattern using a partial figure “8” loop between post or cleats 10506 and 10508. Other winding or loop patterns can also be used including various combinations of zig-zag arrangements.

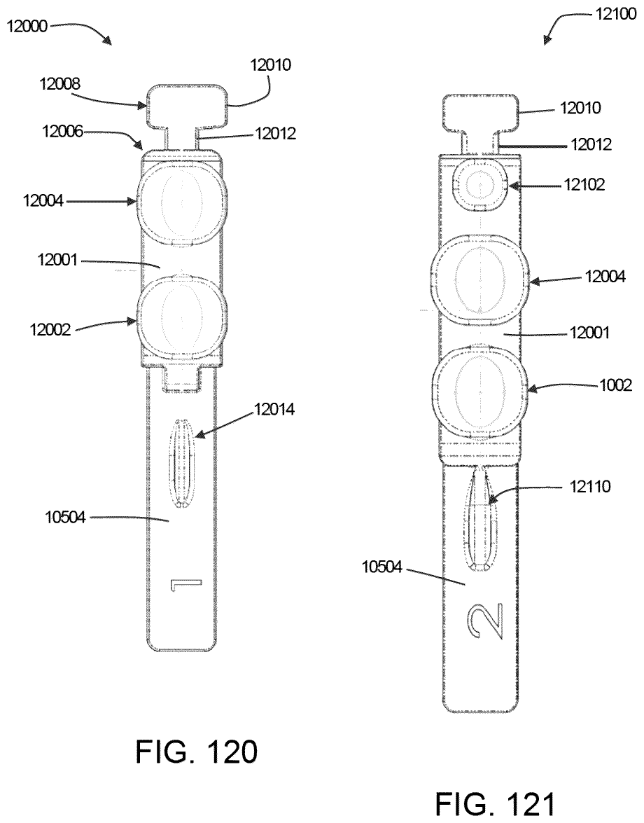

FIGS. 120 and 121 illustrate additional implementations of a connector device. FIG. 120 illustrates a connector 12000 having a secondary, elevated base 12001 and two posts or cleats 12002 and 12004. A third post or cleat 12008 is provided extending longitudinally from an end portion 12006 of the connector body and can be used for terminating the wire/actuation element(s). Posts or cleats 12002 and 12004 can be according to any of the disclosed arrangements. Post or cleat 12008 can be similarly arranged and includes, for example, head portion 12010 and a narrower neck portion 12012. In one implementation, post or cleat 12008 can extend in an orthogonal direction (e.g., approximately 90 degrees) compared posts or cleats 12002 and 12004. Connector 12000 further includes opening 12014 in its body for passage of one or more wires or actuation elements (e.g., 10902). Opening 12014 is approximately centrally located within the width of the connector body (compared to opening 10542 shown approximately to one side of the connector body in FIG. 105.) FIG. 121 illustrates a connector implementation 12100 similar to connector 12000, except that an additional post or cleat 12102 is provided, which can be smaller (or larger) than the other cleats. Connector implementation 12100 also includes opening 12110 approximately centrally located within the wide of the connector body.

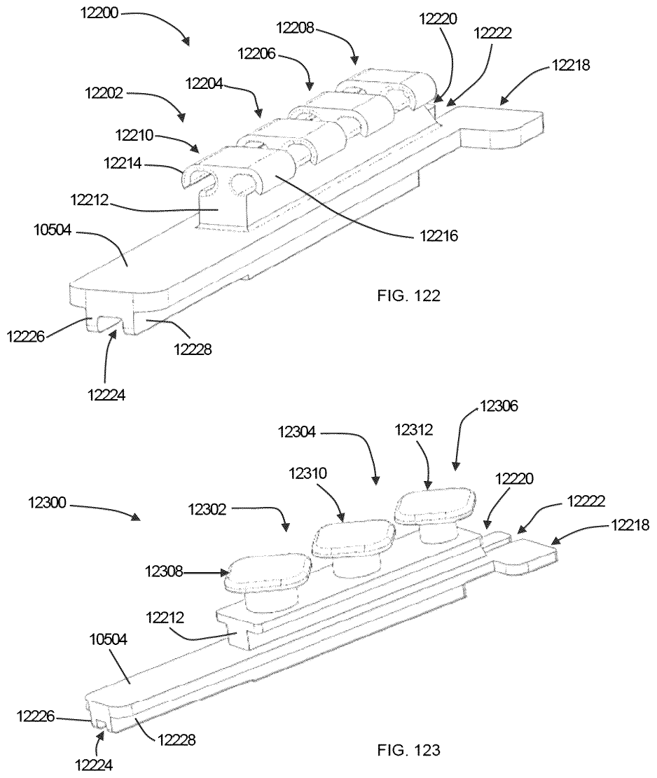

FIG. 122 illustrates another implementation 12200 of a connector device. Implementation 12200 includes post or cleat arrangement having curved or flared head portions. For example, implementation 12200 include a plurality of post or cleats 12202, 12204, 12206 and 12208. Each post or cleat can include, for example, a curved or flared head portion 12210. One or more of the sides 12214 and 12216 can extend from a central mounting portion and include a curved or flared section, which may resemble a mushroom shape. Such curved or flared sides 12214 and 12216 create inner or recessed spaces therein that allow for one or more wires or actuation elements to pass through or be wound or looped there around. A second base portion 12212 can be provided on which the one or more posts or cleats are arranged. The connector body can also include a post or cleat that extends longitudinally from the connector body such as, for example, posts or cleats 12218 and 12220. A gap or slot 12222 can also be provided between the longitudinally extending posts or cleats 12218 and 12220 through which or around which one or more wires or actuation elements can be arranged and terminated by winding. The connector body can also include a mounting arrangement having a channel 12224 and one or more side walls 12226 and 12228 for mounting the connector to a follower (as previously described).

FIG. 123 illustrates another implementation 12300 of a connector device. Implementation 12300 is similar to implementation 12200, except that posts or cleats 12302, 12304, and 12306 having generally diamond shaped (or generally square or rectangular) head portions 12308, 12310, and 12312.

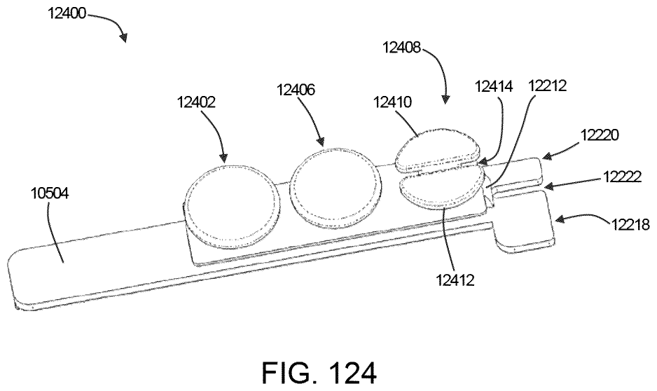

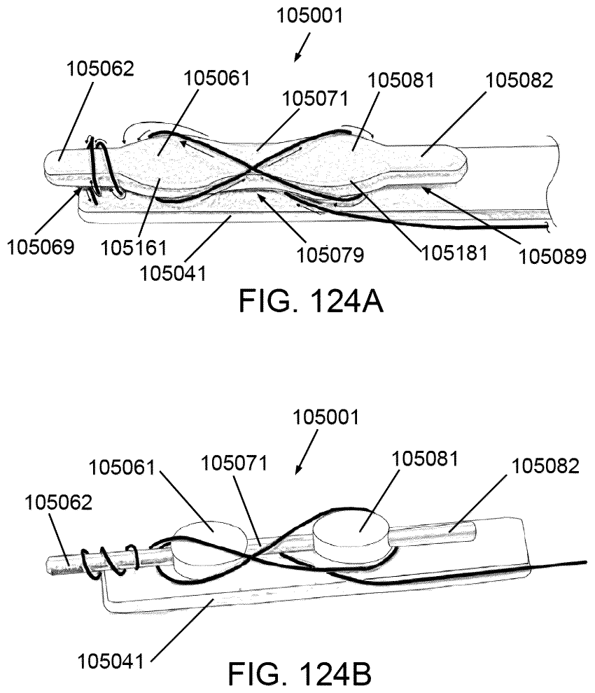

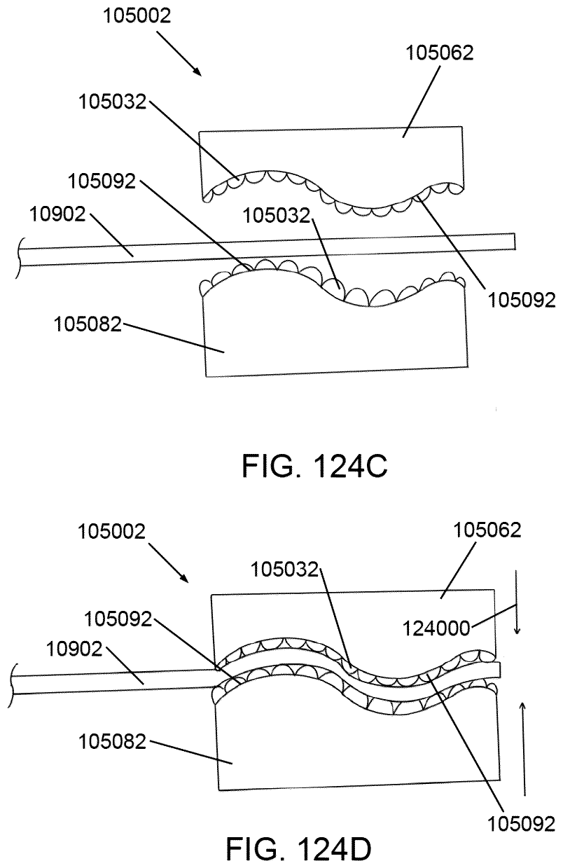

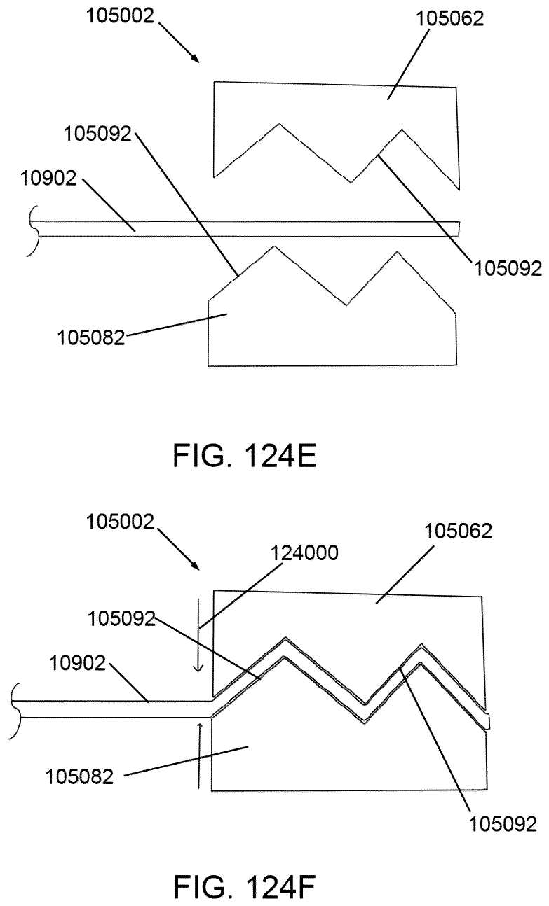

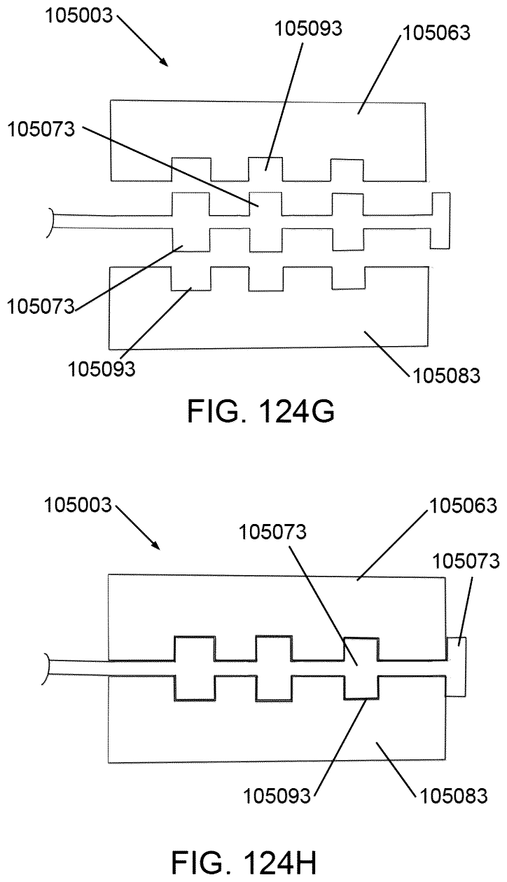

FIG. 124 illustrates another implementation 12400 of a connector device. Implementation 12400 is similar to implementation 12300, except that the posts or cleats having approximately circular head portions 12402, 12406 and 12408. FIG. 124 also illustrates that one or more of the posts or cleats (or even head portions) can be arranged in halves 12410 and 12412 with a space or gap 12414 therebetween. In other implementations, the head portion can be a body having a slot therein. Further, the head portion can include a body having first and second portions separated by a slot or gap (as shown).

Hence, various arrangement of connector devices can be implemented with various configurations of posts or cleats. Also, various winding or looping patterns can be used to connect one or more wires or actuation elements to the connector devices. The connector devices can be made from any suitable material such as metal (e.g., stainless steel, nitinol, etc.), polymers, and/or plastics, and can manufactured by any suitable process including, for example, 3D printing, casting, machining, molding, sintering, etc. Moreover, the connector devices can be formed from one or more components affixed to each (e.g., such as by welding, soldering, glueing, etc.)

FIGS. 124A-124H illustrate additional examples of the wide variety of forms that the connector devices can take. In the examples illustrated by FIGS. 124A and 124B, connector 105001 includes a body having a base portion 105041 and one or more projections or posts 105061, 105081 arranged thereon. In the examples of FIGS. 124A and 124B, a bridge 105071 extends between the projections or posts 105061, 105081, an extension 105062 extends from projection or post 105061, and an extension 105082 extends from projection or post 105081. The bridge 105071, extension 105062, and extension 105082 can take a variety of different forms. In the example illustrated by FIG. 124A, the bridge 105071, the extension 105062, and the extension 105082 are integrally formed with the projection or post 105061 and the projection or post 105081. In the example illustrated by FIG. 124B, the bridge 105071, the extension 105062, and the extension 105082 are formed from a pin 105091 that extends through the projection or post 105061 and the projection or post 105081.

The body 105041 can include support portion that can take the form of the support portion of any of the connectors disclosed herein. The body 105041 is used to mount connector 105001 to, for example, a follower device such as follower 5506.

In the example illustrated by FIG. 124A, posts or cleats 105061, 105081 can have optional head portions 105161, 105181. One or more wire or actuation elements can be wound around the connector 105001 in order to connect the wire or actuation elements to the connector. Gaps 105069, 105089, 105079 can be provided between the support portion 105361 and the extension 105062, the extension 105082, and the bridge 105071. The gaps 105069, 105089, 105079 provide guidance during winding and retention both during and after winding of the one or more wires or actuation elements to the connector 105001.

The wire(s) or actuation elements 10902 can be wound around the connector 105001 in a variety of different ways. For example, the wire(s) or actuation elements 10902 can be wound in any of the ways described in this patent application. In the example illustrated by FIG. 124A, the wire(s) or actuation elements 10902 can be wound in the path/sequence/order indicated by the arrows. When two wires or actuation elements are included, one wire or actuation element can be terminated on the first extension 105062 (as illustrated) and one wire or actuation element can be terminated on the second extension 105082.

In the examples illustrated by FIGS. 124C-124F, a connector 105002 includes a first clamp half 105062 and a second clamp half 105082. The clamp halves can take a variety of different forms. In the examples illustrated by FIGS. 124C-124F, each clamp half 105062, 105082 includes a complimentary tortuous surface 105092. The tortuous surface 105092 can take a variety of different forms. Any shape that deforms the wire(s) or actuation elements 10902 to secure them in the connector 105002 can be used. In the example illustrated by FIGS. 124C and 124D, the tortuous surface 105092 has a smooth, curved, or sinuous shape. In the example illustrated by FIGS. 124E and 124F, the tortuous surface 105092 has a jagged, sharp, or biting shape. In some implementations, features of the tortuous surface 105092 of FIGS. 124C and 124D can be combined with features of the tortuous surface 105092 of FIGS. 124E and 124F.

In some implementations, the tortuous surface 105092 can be provided with optional grip enhancing features 105032 (see FIGS. 124C and 124D) that enhance a grip or bite on the wire(s) or actuation elements 10902. The grip enhancing features 105032 can take a variety of different forms. For example, the grip enhancing features 105032 can comprise knurling, roughening, abrading, projections, recesses, etc.

FIGS. 124C and 124E illustrate the connectors 105002 in open or separated conditions. The wire(s) or actuation element(s) 10902 can be placed in the open connectors. Referring to FIGS. 124D and 124F, the connectors are moved to a closed condition (as indicated by arrows 124000) where the first clamp half 105062 and a second clamp half 105082 secure the wire(s) or actuation element(s) 10902. In the examples illustrated by FIGS. 124D and 124F, the first clamp half 105062 and the second clamp half 105082 deform the wire(s) or actuation element(s) 10902 to the shape of the complimentary tortuous surface 105092. The wire(s) or actuation element(s) 10902 can be plastically or elastically deformed to the shape of the complimentary tortuous surface 105092.

The connectors 105002 can be used in place of any of the connectors disclosed herein and can have any of the features of any of the connectors disclosed herein. For example, the features of the base that allow the connector to be coupled to a driving component can be included on the connectors 105002.

In the examples illustrated by FIGS. 124G-124H, a connector 105003 includes a first clamp half 105063 and a second clamp half 105083. In this example, the wire(s) or actuation elements 10902 include connection features 105073. The connection features 105073 can take a variety of different forms. For example, the connection features can be projections or protuberances that extend from the wire(s) or actuation elements 10902, beads or other structures that extend from the wire(s) or actuation elements 10902, material added to the wire(s) or actuation elements 10902 by, for example, welding, soldering, brazing, etc.

The clamp halves can take a variety of different forms. In the examples illustrated by FIGS. 124G-124H, each clamp half 105063, 105083 includes one or more recesses 105093 that are complimentary to the connection features 105073. The one or more recesses 105093 can take a variety of different forms. Any shape that mates with the connection features 105073 to hold the wire(s) or actuation elements 10902 relative to the connector 105003 can be used.

FIG. 124G illustrates the connector 105003 in an open or separated condition. The wire(s) or actuation element(s) 10902 can be placed in the open connectors. Referring to FIG. 124G, the connector is moved to a closed condition where the first clamp half 105063 and the second clamp half 105083 secure the connection features 105073 in the recesses 105093 to secure the wire(s) or actuation element(s) 10902 to the connector 105003. The connection features 105073 can optionally be deformed when the connector is moved to the closed condition.

The connectors 105003 can be used in place of any of the connectors disclosed herein and can have any of the features of any of the connectors disclosed herein. For example, the features of the base that allow the connector to be coupled to a driving component can be included on the connectors 105002.

FIGS. 125-127 illustrate an example seal assembly 5631 for a handle assembly of a steerable catheter assembly. The seal assembly 5631 can include a seal housing 5635, a seal member 5637 disposed in the seal housing 5635, and cap 5633 for engaging the seal housing 5635. The seal assembly 5631 can be used with the handle assembly 4700 shown in FIGS. 68-89, or the seal assembly 5631 can be used with any other suitable handle assembly of a steerable catheter assembly.

Referring to FIG. 125, the seal housing 5635 defines an interior 5639 that is configured to receive the seal member 5637. The seal housing 5635 can be integral to a guide member or rail (e.g., any guide member or rail described in the present application) or a separate component that is attached to a guide member or rail or other component of a handle assembly. The seal housing 5635 can be made of, for example, plastic.

In some implementations, the seal housing 5635 can include one or more alignment members 5641 for alignment of the cap 5633 (FIGS. 126-127) relative to the seal housing 5635. In the illustrated example, the seal housing 5635 includes three alignment members 5641, but it should be understood that the housing 5635 can include any other suitable number of alignment members. The illustrated example shows the alignment members 5641 being evenly distributed about the housing 5635. As the illustrated example includes three alignment members 5641, the alignment members 5641 are positioned at an angle of 120 degrees relative to an adjacent alignment member 5641.

In some implementations, as illustrated, the alignment members 5641 are ribs that are configured to engage with alignment slots 5643 (FIG. 126) of the cap 5633. In some implementations, the alignment members 5641 can include openings or slots that are configured to engage with ribs of the cap 5633.

Referring to FIG. 126, the cap 5633 includes one or more connection elements 5645 for connecting the cap 5633 to the seal housing 5635. In the illustrated example, the connection elements 5645 include snap connectors that are configured to connect the cap 5633 to the seal housing 5635 by a snap connection. However, it should be understood that the cap 5633 can be connected to the seal housing 5635 by any other suitable means. In the illustrated implementation, the cap 5633 is shown as having six connection elements 5645, but it should be understood that the cap 5633 can have any other suitable number of connection elements.

Still referring to FIG. 126, the cap 5633 includes one or more alignment slots 5643 that are configured to engage with the alignment members 5641 of the seal housing 5635. The alignment slots 5643 can be configured to receive the alignment members 5641 to secure the cap 5633 to the seal housing 5635 in a desired position. The engagement between alignment members 5641 and the alignment slots 5643 can be configured to prevent rotation of the cap 5633 relative to the housing 5635.

In some implementations, as shown in the illustrated example, the cap 5633 includes six alignment slots 5643, but it should be understood that the cap 5633 can include any other suitable number of alignment slots. The illustrated example shows the alignment slots 5643 being evenly distributed about the cap 5633. As the illustrated example includes six alignment slots 5643, the alignment slots 5643 are positioned at an angle of 60 degrees relative to an adjacent alignment slot 5643. Referring to FIGS. 125 and 126, in the illustrated example, the number and positioning of both the alignment members 5641 and the alignment slots 5643 allows for the cap 5633 to be attached to the seal housing 5635 in various positions. That is, the cap 5633 can be rotated by at least 60 degrees in each direction relative to the seal housing 5635 and the alignment members 5641 and alignment slots 5643 will allow for the cap 5633 to be secured to the housing 5635. In some implementations, three of the alignment slots 5643 align with the three alignment members 5641 when the cap 5633 is attached to the seal housing 5635.

Referring to FIGS. 126 and 127, the cap 5633 can include a main opening 5649 through a face 5647 that aligns with a main opening 5651 (FIG. 125) of the seal member 5637 when the cap 5633 is connected to the seal housing 5635. Referring to FIG. 127, the cap 5633 can include a cut out portion 5653 and/or openings 5655 that surround the main opening 5649 that can assist in aligning the cap 5633 with the seal housing 5635 in a desired position. For example, in the illustrated example, the cut-out portion 5653 has a hexagon shape where each straight-line portion of the hexagon shape aligns with an alignment slot 5643, which allows a user to determine the position of the alignment slots 5643 relative to the seal housing 5635 when attaching the cap 5633 to the seal housing.

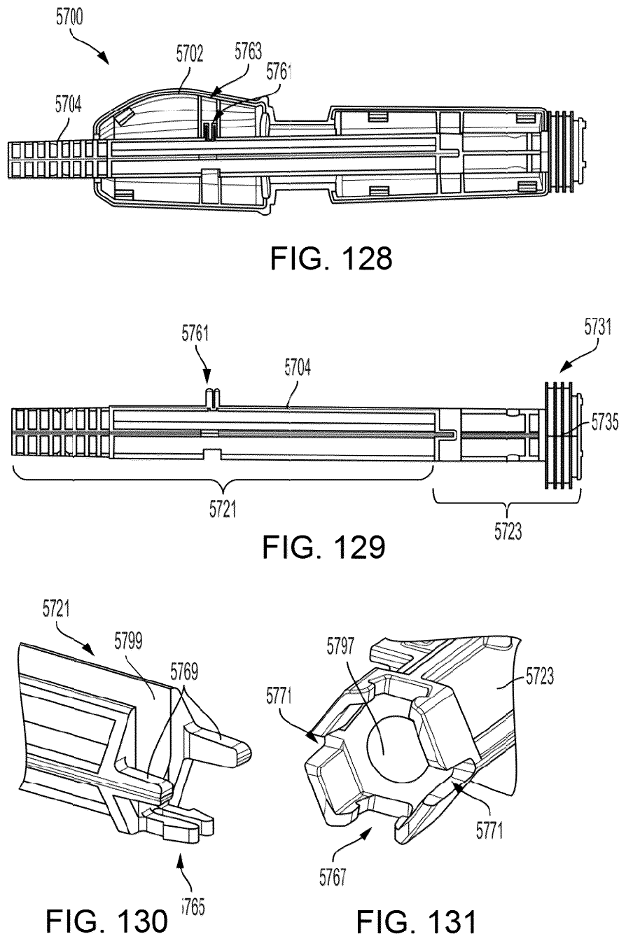

FIGS. 128-131 illustrate an example guide member 5704 or rail disposed within an example housing 5702 for a handle assembly 5700 of a steerable catheter assembly. The member 5704 or rail can take any suitable form and include any suitable features, such as, for example, the form and features of the guide member 4704 or rail described with reference to FIGS. 68-85. The housing 5702 can take any suitable form and include any suitable features, such as, for example, the form and features of the housing 4702 described with reference to FIGS. 68-89.

In some implementations, the guide member 5704 can include a stop member 5761 that is configured to be engaged by a slot 5763 of the housing 5702 to prevent longitudinal movement of the guide member 5704 relative to the housing 5702.

Referring to FIG. 129, the guide member 5704 can have a distal portion 5721 and a proximal portion 5723, where the distal portion 5721 and proximal portion 5723 are configured to be connected to create the guide member 5704. In the illustrated example, a seal housing 5735 of a seal assembly 5731 is integral to the proximal portion 5723 of the guide member 5704. The seal housing 5735 and seal assembly 5731 can take any suitable form, such as, for example, the form of the seal housing 5635 and seal assembly 5631 described with reference to FIGS. 125-127.

Referring to FIGS. 130 and 131, in some implementations, the distal portion 5721 of the guide member 5704 includes a channel 5799 and the proximal portion 5723 includes a lumen or passage 5797. The channel 5799 can make molding of the proximal portion 5721 easier, reduce the material needed to make the proximal portion 5721, and/or make assembly of the catheter with the guide member 5704 easier. The lumen or passage 5797 accepts the catheter. The catheter is hermetically sealed in the lumen or passage 5797, such that a fluid tight passage is formed between the seal assembly 5731 and the catheter. As such, the catheter can be flushed through the flush port that is attached to the seal assembly 5631.

Referring to FIGS. 129-131, the distal portion 5721 of the guide member 5704 can have one or more connection elements 5765 for engaging with one or more connection elements 5767 of the proximal portion 5723 to connect the distal and proximal portions 5721, 5723 together. In some implementations, the connection element 5765 of the distal portion 5721 can include extending portions, and the connection element 5767 of the proximal portion 5723 can include an opening or slot, where the extending portions of the connection element 5765 are configured to extend into the opening or slot of the connection element 5767 such that the distal and proximal portions 5721, 5723 connect to each other by a snap connection. In some implementations, the connection portion 5765 of the distal portion 5721 can include an opening or slot for receiving extending portions of the connection portion 5767 of the proximal portion 5723 such that the distal and proximal portions 5721, 5723 can be connected by a snap connection.

The connection features 5765, 5767 can take any other suitable form that allow for the distal and proximal portions 5721, 5723 to be connected together. In some implementations, the distal and proximal portions 5721, 5723 can be welded or otherwise bonded together after the connection elements 5765, 5767 are connected together.

In some implementations, as shown in the illustrated example, the distal portion 5721 includes protruding members 5769 that are configured to be received by slots 5771 of the proximal portion 5723 to further assist in securing the distal and proximal portions 5721, 5723 together. In some examples, the proximal portion 5721 can include protruding members (not shown), and the distal portion 5721 can include slots for receiving the protruding members to further assist in securing the distal and proximal portions 5721, 5723 together.

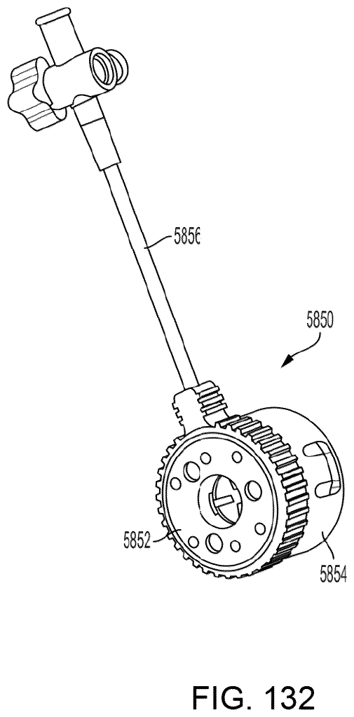

Referring now to FIGS. 132-135, a schematic of an example of a catheter coupler 5850 is illustrated. In some implementations, the catheter coupler 5850 can be coupled to at least one of an implant pusher or implant catheter, a device catheter, a steerable catheter, an outer sheath, or any other suitable member of a delivery device. For example, the catheter coupler 5850 can be used with the handle assembly 4700 for a steerable catheter shown in FIGS. 68-89, or with any other catheter assembly described in the present application. Each catheter coupler can be connected to a control handle (e.g., handle assembly 4700 shown in FIGS. 68-89) that controls operation/positioning of a steerable catheter or any other suitable catheter. The catheter coupler 5850 can be used to sense or monitor fluid pressure in the catheter and/or can be used to flush the catheter, such that no air is present in the catheter.

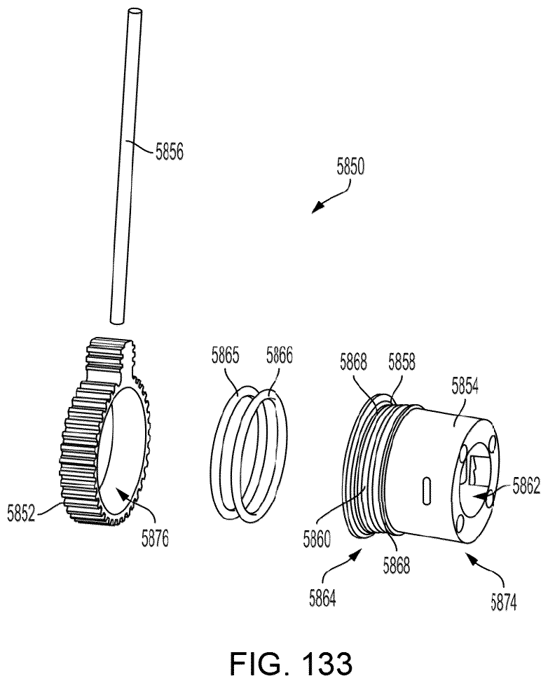

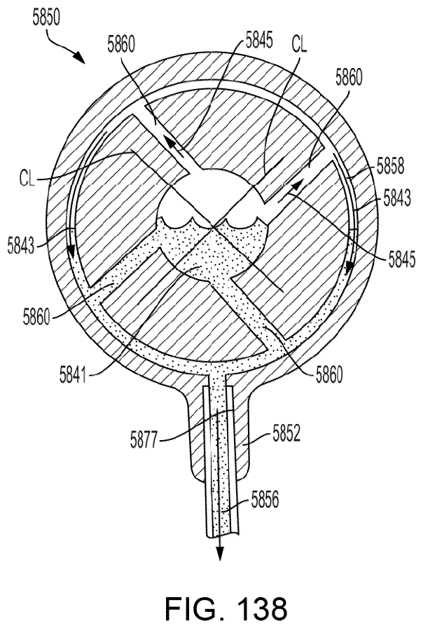

The catheter coupler 5850 can take a wide variety of different forms. In the examples shown in FIGS. 133-137, the catheters couplers are configured such that the cap 5852 and flush tube 5856 are configured to rotate relative to the housing 5854 and the attached catheter. In the example illustrated by FIG. 138, the catheter coupler is configured to allow flushing of the catheter, without rotating the cap 5852 and attached flush tube 5856. The examples described below are two of the ways that catheter couplers can be configured to allow flushing of the catheter, without rotating the catheter (i.e., flushing by rotating the flush tube to top-dead-center and flushing without needing to rotate the flush tube).

With reference to FIGS. 132-133 an example of a catheter coupler 5850 is shown. The illustrated coupler 5850 includes a cap 5852 that is rotatably mounted to a housing 5854. The cap 5852 can be coupled to a tube 5856. The tube 5856 can be used for a variety of different purposes. For example, the tube 5856 can be used to flush the catheter, measure pressure in the catheter, sample fluids from the catheter, deliver fluid through the catheter, etc.

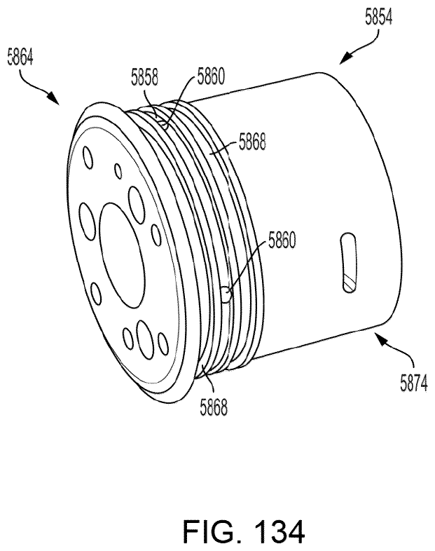

With reference to FIG. 133-134, in some implementations, the housing 5854 can include a fluid channel 5858 disposed circumferentially around the housing 5854. The channel 5858 is illustrated in the housing but can be defined or partially defined in the cap 5852. The channel 5858 is connected to at least one port 5860. The port 5860 connects the channel 5858 to a lumen or central passage 5862 of the housing 5854. The lumen or central passage 5862 extends between the first end 5864 and the second end 5874 of the housing 5854.

In some implementations, the cap 5852 is rotatably attached to the housing 5854 at the first end 5864 of the housing 5854. The cap 5852 is ring shaped with a central opening 5876. A lumen or passage 5877 extends from the central opening 5876 of the cap 5852 to the tube 5856. As a result, fluid inside the central opening 5876 can flow through the cap to the tube 5856.

In some implementations, the cap 5852 can fit over top the first end 5864 of the housing 5854 such that a seal is formed between the cap 5852 and the housing 5854 on both sides of the channel 5858. The seals between the cap 5852 and the housing 5854 can be formed in a variety of ways.

In some implementations, as illustrated in FIG. 133, the catheter coupler 5850 can include one or more sealing members. For example, catheter coupler 5850 can include a first sealing member 5865 and a second sealing member 5866. The sealing members can be ring-shaped and fit between the housing 5854 and the cap 5852. In some implementations, the first sealing member 5865 and a second sealing member 5866 fit in grooves 5868, which are set in the housing 5854. The seals 5865, 5866 prevent any fluid in the channel 5858 from escaping through the rotatable coupling between the cap 5852 and the housing 5854. As a result, fluid in the passage 5862 can flow through the port 5860, into the channel 5858, through the passage 5877, and through the tube 5856 (or vice versa), without leakage between the cap 5852 and the housing 5854.

In some implementations, the cap 5852 can rotate with respect to the housing 5854. For example, in some implementations, the cap 5852 can rotate 360 degrees about the housing 5854 and can rotate clockwise and/or counterclockwise with respect to the housing 5854. The sealing members 5865, 5866 are configured to maintain seals between the housing 5854 and the cap 5852 while the cap rotates relative to the housing.

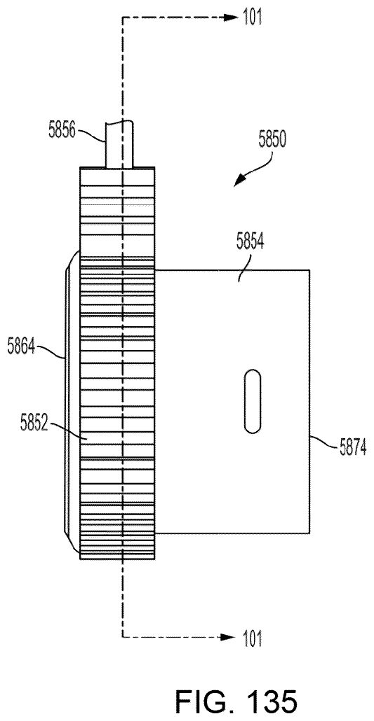

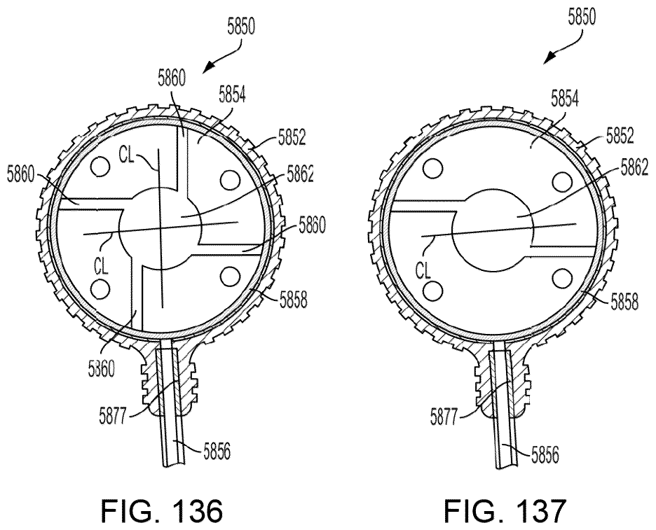

FIG. 136 illustrates a cross section of an implementation of the catheter coupler 5850, taken along the plane indicated by lines 101-101 in FIG. 135. In the illustrated example, the housing 5854 includes four passages 5860 that connect the lumen or passage 5862 to the circumferential channel 5858. The housing 5854 can have any suitable number of passages 5860 connecting the lumen or passage 5862 to the channel or groove 5858. The tube 5856 is in fluid communication with the channel 5858, the passages 5860, and the lumen or passage 5862.

In some implementations, as shown in the illustrated example, the passages 5860 are offset from the corresponding center lines CL of the lumen or passage 5862. This offset positioning of the passages 5860 relative to the corresponding center lines CL of the lumen or passage 5862 is configured to create a fluid vortex to assist in flushing out any air within the system. That is, the fluid vortex is configured to remove air bubbles that are stuck within the system.

FIG. 137 illustrates a cross section of another implementation of the catheter coupler 5850, taken along the plane indicated by lines 101-101 in FIG. 135. In the illustrated example, the housing 5854 includes two passages 5860 that connect the lumen or passage 5862 to the circumferential channel 5858. The housing 5854 can have any suitable number of passages 5860 connecting the lumen or passage 5862 to the channel or groove 5858. The tube 5856 is in fluid communication with the channel 5858, the passages 5860, and the lumen or passage 5862.

In the illustrated example, the passages 5860 are offset from the center lines CL of the lumen or passage 5862. This offset positioning of the passages 5860 relative to the center lines CL of the lumen or passage 5862 is configured to create a fluid vortex to assist in flushing out any air within the system. That is, the fluid vortex is configured to remove air bubbles that are stuck within the system.

FIG. 138 illustrates a cross section of another implementation of the catheter coupler 5850, taken along the plane indicated by lines 101-101 in FIG. 135. In the illustrated example, the housing 5854 includes four passages 5860 that connect the lumen or passage 5862 to the circumferential channel 5858. The housing 5854 can have any suitable number of passages 5860 connecting the lumen or passage 5862 to the channel or groove 5858. The tube 5856 is in fluid communication with the channel 5858, the passages 5860, and the lumen or passage 5862.

In some implementations, as shown in the illustrated example, the passages 5860 are offset from the corresponding center lines CL of the lumen or passage 5862. This offset positioning of the passages 5860 relative to the corresponding center lines CL of the lumen or passage 5862 is configured to create a fluid vortex to assist in flushing out any air within the system. That is, the fluid vortex is configured to remove air bubbles that are stuck within the system.

The viscosity of air is less than the viscosity of a liquid, such as water (i.e., saline) and/or blood. Therefore, the air has lower resistance to fluid flow. The passages with the lowest fluid flow resistance (i.e., the upper ones of the passages 5860 and the upper portion of the circumferential passage 5858 containing air) will see the largest total volume (air+liquid) flow through them when a vacuum is applied to the tube 5856. If the potential created by the vacuum applied to the tube 5856 (the “net positive suction head applied” or “NPSHA”) is greater than the height potential between the top and bottom passages 5860 (the “net positive suction head required” or “NPSHR”), the fluid can flow first and most rapidly through the air exposed ports, resulting in evacuation of the air from the seal housing regardless of the flush tube orientation as illustrated by FIG. 138.

Still referring to FIG. 138, a user can draw a vacuum through the tube 5856, which pulls fluid 5841 in the catheter into the passage 5862. The liquid 5841, such as flush fluid and/or blood in the passage 5862 displaces air in the passage 5862 through the upper ports or passages 5860 as illustrated by arrows 5843.

In some implementations, the circumferential passage 5858 and/or the ports or passages 5860 can be sized such that air or a mixture of air and liquid 5841 in the upper ones of the passages 5860 and the circumferential passage 5858 flows to the outlet port 5877 before or faster than the liquid in the lower ports flows to the outlet port 5877. This preferential flow is regardless of the orientation of the coupler 5850 and fixed outlet port. That is, the circumferential passage 5858 and/or the ports or passages 5860 are small enough or constrictive enough to allow air or a mixture of air and liquid 5841 in the upper passages and the circumferential passage 5858 to flow to the outlet port 5877 before or faster than the liquid in the lower ports flows to the outlet port 5877, regardless of the orientation of the coupler 5850 and fixed outlet port.

In some implementations, the circumferential passage 5858 and/or the ports or passages 5860 can be sized for this preferential flow of air or air mixed with the liquid over liquid alone, because air or air mixed with liquid is less viscous than the liquid alone.

In some implementations, a cross-section of the circumferential passage 5858 is substantially rectangular with a cross-sectional width between 0.015 and 0.125 inches, such as between 0.030 and 0.110 inches, such as between 0.050 and 0.100 inches, such as between 0.060 and 0.080 inches, such as about 0.070 inches, such as 0.070 inches, and a height between 0.010 and 0.100 inches, such as between 0.020 and 0.80 inches, such as between 0.025 and 0.050 inches, such as between 0.030 and 0.040 inches, such as about 0.035 inches, such as 0.035 inches, and the ports or passages are substantially circular with a cross-sectional diameter between 0.015 and 0.125 inches, such as between 0.030 and 0.110 inches, such as between 0.050 and 0.100 inches, such as between 0.060 and 0.080 inches, such as about 0.070 inches, such as 0.070 inches to facilitate the preferential flow of air and air mixed with the liquid over the liquid alone.

When the circumferential passage 5858 and/or the ports or passages 5860 are appropriately sized, the air, can more readily travel from the lumen or passage 5862, out the upper passages as indicated by the arrows 5845, along the circumferential passage 5858 as indicated by arrow 5843, and out through the outlet 5877 and tube 5856. During use, the liquid 5841 begins to fill the upper ones of the lumens or passages 5860, forcing the air into the circumferential passage 5858. The air and air mixed with liquid continues to move along the circumferential passage 5858 as indicated by arrow 5843, and out through the outlet 5877 and tube 5856.

If the circumferential passage 5858 and/or the ports or passages 5860 were two large, there would be less restriction on the liquid flowing through the lower portion of the circumferential passage 5858 and/or lower ones of the ports or passages 5860. As a result, the preferential flow of the air or the air and liquid mixture over the liquid alone would not occur. In some examples, a larger vacuum can be applied to the tube if the vacuum applied for given sizes of the circumferential passage 5858 and the ports or passages 5860 does not result in the preferential flow of the air out of the catheter. However, large sizes of the circumferential passage 5858 and/or the ports or passages can prevent any reasonable vacuum force from preferentially withdrawing the air out of the catheter.

Still referring to FIG. 138, the tube 5856 can be used for a variety of different purposes. For example, the tube can be used to flush the catheter, measure pressure in the catheter, sample fluids from the catheter, deliver fluid through the catheter, etc.

### EXAMPLES

Example 1. A catheter assembly for a transvascular delivery system comprising:
a handle housing; and
a sheath extending longitudinally from the handle housing;
wherein the sheath comprises a coil that includes one or more supporting elements and one or more tubes that define a main lumen, wherein each of the one or more tubes defines a lumen; and
wherein the lumen of each of the one or more tubes is configured receive a control element such that the control element can be moved in a direction from a proximal portion of the sheath to a distal portion of the sheath.

Example 2. The catheter assembly according to example 1, wherein each tube of the one or more tubes comprises a hypotube.

Example 3. The catheter assembly according to example 2, wherein the hypotube is made from nitinol.

Example 4. The catheter assembly according to any one of examples 1-3, wherein the one or more tubes of the coil include at least two tubes.

Example 5. The catheter assembly according to any one of examples 1-4, wherein at least a portion of the coil includes the one or more supporting elements and the one or more tubes being coiled in a clockwise direction.

Example 6. The catheter assembly according to example 5, wherein the at least a portion of the coil has a pitch angle of between about 2 degrees and about 80 degrees.

Example 7. The catheter assembly according to example 5, wherein another portion of the coil includes the one or more supporting elements and the one or more tubes being in a straight configuration.

Example 8. The catheter assembly according to any one of examples 1-7, wherein the sheath further comprises an outer coil layer that is positioned over the coil, wherein the outer coil layer comprises a plurality of supporting elements.

Example 9. The catheter assembly according to example 8, wherein the plurality of supporting elements of the outer coil layer are coiled in a counterclockwise direction, and wherein at least a portion of the coil includes the one or more supporting elements and the one or more tubes being coiled in a clockwise direction.

Example 10. The catheter assembly according to any one of examples 1-9, wherein the control element comprises a wire.

Example 11. The catheter assembly according to any one of examples 1-10, further comprising a handle assembly that includes the handle housing, a guide member, a follower that is movably connected to the guide member, and a driver that is engageable by the user to move the follower relative to the guide member.

Example 12. The catheter assembly according to example 11, wherein the handle housing comprises at least one window that allows for external threads of the follower to be engaged by internal threads of the driver such that the driver can move the follower relative to the guide member.

Example 13. The catheter assembly according to example 11, wherein the follower comprises an indicator and the handle housing comprises an indicator window that allows for the indicator of the follower to be visible to a user during use of the catheter assembly.

Example 14. The catheter assembly according to example 11, wherein the handle assembly further comprises an actuation member that is attached to the driver such that a user can engage the actuation member to cause the driver to engage the follower and move the follower relative to the guide member.

Example 15. The catheter assembly according to example 11, wherein the guide member has a hexagonal shape.

Example 16. The catheter assembly according to example 15, wherein handle housing has one or more walls that are shaped to engage with the hexagonal shape of the guide member to prevent the guide member from rotating relative to the housing.

Example 17. The catheter assembly according to example 11, wherein the handle housing comprises two or more components that are configured to be connected together to create the handle housing.

Example 18. The catheter assembly according to example 11, wherein the guide member comprises two or more components that are configured to be connected together to create the guide member.

Example 19. The catheter assembly according to example 11, further comprising an actuation element that is connected to the sheath and the follower of the handle assembly such that movement of the follower relative to the guide member of the handle assembly causes the actuation element to engage the sheath and move the sheath between a flexed configuration and an unflexed configuration.

Example 20. The catheter assembly according to example 19, wherein the handle assembly further comprises a connector that attaches the actuation element to the follower.

Example 21. The catheter assembly according to example 20, wherein the actuation element is attached to the connector by a stitched connection.

Example 22. The catheter assembly according to example 21, wherein the connector comprises a strain relief portion and a connection portion, wherein the strain relief portion comprises one or more strain relief members for engaging the actuation element to reduce strain applied to the actuation element as a force is applied to the actuation element during use of the catheter assembly, and wherein the connection portion is configured to receive the actuation element such that the actuation element is connected to the connector by the stitched connection.

Example 23. A catheter assembly for a transvascular delivery system, the catheter assembly comprising:
a handle assembly that comprises:
   a handle housing;
   a guide member at least partially disposed within the handle housing;
   a follower movably attached to the guide member;
   a driver that is configured to engage the follower to cause the follower to move relative to the guide member;
   a steerable catheter extending from the handle assembly, wherein the steerable catheter is movable between a flexed configuration and an unflexed configuration; and
   an actuation element connected to the follower and the steerable catheter;
wherein movement of the follower relative to the guide member causes the actuation element to engage the steerable catheter to move the steerable catheter between the flexed configuration and the unflexed configuration.

Example 24. The catheter assembly according to example 23, wherein the handle housing comprises two or more components that are configured to be connected to create the handle housing.

Example 25. The catheter assembly according to any one of examples 23-24, wherein the guide member comprises two or more components that are configured to be connected to create the guide member.

Example 26. The catheter assembly according to any one of examples 23-25, wherein the follower comprises a body member and threads extending outward from the body member, wherein the threads are configured to be engaged by threads of the driver to move the follower relative to the guide member.

Example 27. The catheter assembly according to example 26, wherein handle housing comprises at least one window that allows for the threads of the follower to be engaged by threads of the driver such that the driver can move the follower relative to the guide member.

Example 28. The catheter assembly according to any one of examples 23-27, wherein the follower comprises an indicator, and wherein the handle housing has an indicator window that allows for the indicator of the follower to be visible to a user during use of the catheter assembly.

Example 29. The catheter assembly according to any one of examples 23-28, wherein the handle assembly further comprises an actuation member that is attached to the driver such that a user can engage the actuation member to cause the driver to engage the follower and move the follower relative to the guide member.

Example 30. The catheter assembly according to any one of examples 23-29, wherein the guide member has a hexagonal shape.

Example 31. The catheter assembly according to example 30, wherein handle housing has one or more walls that are shaped to engage with the hexagonal shape of the guide member to prevent the guide member from rotating relative to the housing.

Example 32. The catheter assembly according to any one of examples 23-31, wherein the actuation element comprises a wire.

Example 33. The catheter assembly according to any one of examples 23-32, wherein the handle assembly further comprises a connector that attaches the actuation element to the follower.

Example 34. The catheter assembly according to example 33, wherein the actuation element is attached to the connector by a stitched connection.

Example 35. The catheter assembly according to example 34, wherein the connector comprises a strain relief portion and a connection portion, wherein the strain relief portion comprises one or more strain relief members for engaging the actuation element to reduce strain applied to the actuation element as a force is applied to the actuation element during use of the catheter assembly, and wherein the connection portion is configured to receive the actuation element such that the actuation element is connected to the connector by the stitched connection.

Example 36. The catheter assembly according to example 35, wherein each of the strain relief members comprise a curved portion for engaging with the actuation element.

Example 37. The catheter assembly according to example 35, wherein at least one of the strain relief members has a circular shape.

Example 38. The catheter assembly according to example 35, wherein the strain relief portion comprises a first opening disposed between a first strain relief member and a second strain relief member of the one or more strain relief members, and a second opening disposed between the second strain relief member and a third strain relief member of the one or more strain relief members.

Example 39. The catheter assembly according to example 35, wherein the actuation element engages the first strain relief member, extends through the first opening, engages the second strain relief member, extends through the second opening, and engages the third strain relief member when the actuation element is attached to the connection portion of the connector.

Example 40. The catheter assembly according to any one of examples 33-39, wherein the connector comprises one or more openings for connecting the actuation element to the connector by the stitched connection.

Example 41. The catheter assembly according to example 40, wherein the connector comprises a support member proximate the one or more openings for engaging the actuation element and reducing the strain placed on the actuation element due to contact with the connector at the one or more openings.

Example 42. The catheter assembly according to example 41, wherein the connector is made of a first material and the support member is made from a second material that is different than the first material.

Example 43. The catheter assembly according to example 41, wherein the connector is made of a metal material and the support member is made of a plastic material.

Example 44. The catheter assembly according to any one of examples 23-43, further comprising a seal assembly having a seal housing, a seal member disposed within the seal housing, and a cap configured to be attached to the seal housing.

Example 45. The catheter assembly according to example 44, wherein the cap comprises a plurality of connection members for attaching the cap to the seal housing.

Example 46. The catheter assembly according to example 45, wherein the plurality of connection members are configured to attach the cap to the seal housing by a snap connection.

Example 47. The catheter assembly according to example 44, wherein the seal housing comprises one or more alignment members and the cap comprises one or more alignment slots for receiving the alignment members of the seal housing.

Example 48. The catheter assembly according to example 47, wherein the one or more alignment members comprise alignment ribs.

Example 49. The catheter assembly according to example 47, wherein the one or more alignment members of the seal housing comprise three alignment members, and wherein the one or more alignment slots of the cap comprise six alignment slots.

Example 50. The catheter assembly according to example 44, wherein the cap comprises a cutout configured to assist a user in aligning the cap with the seal housing.

Example 51. The catheter assembly according to example 50, wherein the cutout has a hexagonal shape.

Example 52. The catheter assembly according to example 44, wherein the seal housing is integral to the guide member.

Example 53. The catheter assembly according to any one of examples 23-52, further comprising a catheter coupler, the catheter coupler comprising:
a catheter connection lumen connected to the steerable catheter;
an outer circumferential passage;
a plurality of connecting passages that each connect the catheter connection lumen to the outer circumferential passage, wherein each of the connecting passages is offset from a center line of the catheter connection lumen; and
an outlet portion in fluid communication with the outer circumferential passage.

Example 54. The catheter assembly according to any one of examples 23-53, further comprising a connector for attaching the actuation element to the follower of the handle assembly, wherein the connector is movably attached to the follower.

Example 55. The catheter assembly according to any one of examples 23-54, wherein the connector is configured to move distally relative to the follower when a user engages the drive to move the steerable catheter from the flexed configuration to the unflexed configuration while a shape of a lumen of the patient in which the steerable catheter is disposed causes the steerable catheter to maintain the flexed configuration.

Example 56. A connector for attaching an actuation element to a handle assembly of a catheter assembly, the connector comprising:
a connector body having a strain relief portion and a connection portion;
the strain relief portion comprising:
   a first strain relief member;
   a second strain relief member;
   a third strain relief member;
   a first opening extending through the connector body and disposed between the first and second strain relief members; and
   a second opening extending through the connector body and disposed between the second and third strain relief members;
the connection portion comprising two or more openings for receiving the actuation element such that the actuation element is attached to the connector body by a stitched connection.

Example 57. The connector according to example 56, wherein the strain relief portion comprises one or more alignment walls for maintaining the actuation wire within the strain relief portion.

Example 58. The connector according to any one of examples 56-57, wherein each of the first, second, and third strain relief members comprises a curved portion for engagement with the actuation element.

Example 59. The connector according to example 58, wherein at least one of the first, second, and third strain relief members has a circular shape.

Example 60. The connector according to any one of examples 56-59, wherein two or more openings of the connection portion comprises at least three openings for receiving the actuation element for attaching the actuation element to the connector body by the stitched connection.

Example 61. The connector according to example any one of examples 56-60, wherein the connection portion comprises two or more second openings for receiving a second actuation element such that the second actuation element is connected to the connector body by a stitched connection.

Example 62. The connector according to any one of examples 56-61, wherein the connector body is made of a metal material.

Example 63. A seal assembly for a catheter assembly, the seal assembly comprising:
a seal housing having one or more alignment members;
a seal member disposed within the seal housing;
a cap configured to attach to the seal housing, the cap having one or more alignment slots that are configured to receive the one or more alignment members of the seal housing.

Example 64. The seal assembly according to example 63, wherein the seal housing comprises three alignment members, each alignment member being positioned 120 degrees from an adjacent alignment member; and wherein the cap comprises six alignment slots, each alignment slot being positioned 60 degrees from an adjacent alignment slot.

Example 65. The seal assembly according to any one of examples 63-64, wherein the cap comprises an alignment cutout configured for assisting a user in connecting the cap to the seal housing.

Example 66. The seal assembly according to example 65, wherein the alignment cutout has a hexagonal shape.

Example 67. The seal assembly according to any one of examples 63-66, wherein the cap comprises a plurality of connection elements that are configured to attach the cap to the seal housing by a snap connection.

Example 68. A catheter coupler, comprising:
a housing having a catheter connection lumen, a plurality of passages, and an outer circumferential channel, wherein the plurality of passages each connect the catheter connection lumen to the outer circumferential channel, wherein each passage of the plurality of passages is offset from a center line of the catheter connection lumen;
a cap rotatably coupled to the housing, wherein the cap has an outlet port in fluid communication with the outer circumferential passage; and
one or more seals that provide seals between the housing and the cap that direct fluid flow from the outer circumferential passage to the outlet port;
wherein the cap is rotatable to position the outlet port a vertical orientation without rotating the housing.

Example 69. A catheter coupler, comprising:
a catheter connection lumen;
an outer circumferential passage;
a plurality of connecting passages that each connect the catheter connection lumen to the outer circumferential passage, wherein each connecting passage is offset from a center line of the catheter connection lumen;
an outlet port in fluid communication with the outer circumferential passage; and
wherein the outer circumferential passage and the plurality of connecting passages are sized such that when:
   the outlet port is oriented in a downwardly facing direction;
   upper ones of the connecting passages contain air; and
   lower ones of the connecting passages contain liquid;
   the air is drawn out of the outlet port.

Example 70. The catheter coupler of example 69 further comprising a catheter attached to the catheter connection lumen.

Example 71. A catheter assembly for a transvascular deliver system comprising:
a patterned tube disposed between an inner and an outer liners, the patterned tube comprising a tube wall having at least first and second pattern openings;
the first pattern openings comprising an interrupted spiral pattern;
the second pattern openings comprising a plurality of flex windows and a plurality of reflow openings disposed opposite the flex windows; and
the plurality of flex windows comprising a transition region having flex windows of different size.

Example 72. The catheter assembly of example 71 wherein the plurality of flex windows comprises flex windows of the same size.

Example 73. The catheter assembly according to any one of examples 71-72 wherein the transition region is disposed proximate the plurality of flex windows having the same size.

Example 74. The catheter assembly according to any one of examples 71-73 wherein the plurality of flex windows comprises a flex region length and the transition region comprises approximately half the flex region length.

Example 75. The catheter assembly according to any one of examples 71-74 wherein the flex windows of different size comprises at least a first flex window having a first size, a second flex window having a second size that is larger than the first size, and a third flex window having a third size that is larger than the second size.

Example 76. The catheter assembly according to any one of examples 71-75 wherein the flex windows of different size comprise a plurality of flex windows having sizes that progressively increase.

Example 77. The catheter assembly according to any one of examples 71-75 wherein the flex windows of different size comprise a plurality of flex windows having sizes that progressively increase at a rate of approximately 110 to 120 percent from window to window.

Example 78. The catheter assembly according to any one of examples 71-75 wherein the flex windows of different size comprise a plurality of flex windows having sizes that progressively decrease.

Example 79. The catheter assembly according to any one of examples 71-75 wherein the flex windows of different size comprise a plurality of flex windows having sizes that progressively decrease at a rate of approximately 80 to 90 percent from window to window.

Example 80. The catheter assembly according to any one of examples 71-79 wherein the patterned tube further comprises a pull ring having a plurality of projecting members, each projecting member comprising a first and second tabs separated by a compressible gap.

Example 81. The catheter assembly according to any one of examples 71-80 wherein the patterned tube further comprises a distal end portion having a plurality of recesses configured for an interference fit connection.

Example 82. A connector for a catheter system, the connector comprising:
a body having a base portion, a plurality of projections extending from the base portion, and a plurality of gaps;
each projection comprising a neck portion and a head portion larger than the neck portion, the neck portion located between the base portion and the head portion; and
each gap located between at least two projections.

Example 83. The connector according to example 82 wherein at least one of the neck portions and at least one of the head portions form a cleat.

Example 84. The connector according to any one of examples 82-83 wherein the neck portion comprises curved walls.

Example 85. The connector according to any one of examples 82-84 wherein the neck portion comprises a tapered center section.

Example 86. The connector according to any one of examples 82-85 wherein the neck portion comprises first and second end sections and a third section between the first and second end sections, and wherein the third section is smaller than the first and section sections.

Example 87. The connector according to any one of examples 82-86 wherein the head portion comprises at least one curved side wall.

Example 88. The connector according to any one of examples 82-87 wherein the head portion has a slot therein.

Example 89. The connector according to any one of examples 82-88 wherein the head portion comprises first and second portions.

Example 90. The connector according to any one of examples 82-89 wherein the plurality of projections comprise at one least projection having a different size than other ones of the plurality of projections.

Example 91. The connector according to any one of examples 82-90 wherein the plurality of projections comprise at least two projections having the same size.

Example 92. The connector according to any one of examples 82-91 wherein the base portion comprises first and second walls separated by a gap.

Example 93. The connector according to any one of examples 82-92 wherein the base portion comprises at least one opening proximate at least one projection.

Example 94. The connector according to any one of examples 82-93 wherein the base portion comprises at least one extension longitudinally extending from the base portion.

Example 95. The connector according to any one of examples 82-94 wherein the base portion comprises a slot longitudinally extending from the base portion.

Example 96. The connector according to any one of examples 82-95 further comprising at least one actuation element having a counterclockwise winding arrangement around the plurality of projections.

Example 97. The connector according to any one of examples 82-95 further comprising at least one actuation element having a clockwise winding arrangement around the plurality of projections.

Example 98. The connector according to any one of examples 82-95 further comprising at least one actuation element having a cross-over winding arrangement around at least one of the plurality of projections.

Example 99. The connector according to any one of examples 82-95 further comprising at least one actuation element having a counterclockwise winding arrangement around at least one of the plurality of projections and a clockwise winding arrangement around at least another one of the plurality of projections.

Example 100. The connector according to any one of examples 82-95 further comprising at least one actuation element having a figure eight winding arrangement around at least two of the plurality of projections.

Example 101. The connector according to any one of examples 82-95 further comprising at least one actuation element having a looped winding arrangement around the plurality of projections.

Example 102. The connector according any one of examples 82-95 further comprising a bridge extending between two head portions.

Example 103. The connector according to example 102 further comprising two extensions extending from the two head portions.

Example 104. A connector assembly for a catheter system, the connector comprising:
an actuation element configured to flex a distal catheter end;
a first clamp half;
a second clamp half;
wherein the actuation element is clamped between the first clamp half and the second clamp half.

Example 105. The connector assembly of example 104 wherein each clamp half includes a tortuous surface that bends the actuation element when the first clamp half is clamped to the second clamp half.

Example 106. The connector assembly of example 105 wherein each clamp half includes a recess that mates with a protuberance of the actuation element.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the examples in the specification.

What is claimed is:

1. A catheter assembly for a transvascular delivery system, the catheter assembly comprising:
a handle assembly that comprises:
a handle housing;
a guide member at least partially disposed within the handle housing;
a follower movably attached to the guide member;
a driver that is configured to engage the follower to cause the follower to move relative to the guide member;
a steerable catheter extending from the handle assembly, wherein the steerable catheter is movable between a flexed configuration and an unflexed configuration;
an actuation element connected to the follower and the steerable catheter; and
wherein movement of the follower relative to the guide member causes the actuation element to engage the steerable catheter to move the steerable catheter between the flexed configuration and the unflexed configuration.

2. The catheter assembly according to claim 1, wherein the follower comprises a body member and threads extending outward from the body member, wherein the threads are configured to be engaged by threads of the driver to move the follower relative to the guide member.

3. The catheter assembly according to claim 2, wherein the handle housing comprises at least one window that allows for the threads of the follower to be engaged by threads of the driver such that the driver can move the follower relative to the guide member.

4. The catheter assembly according to claim 1, wherein the handle assembly further comprises a connector that attaches the actuation element to the follower.

5. The catheter assembly of claim 4 wherein the connector comprises:
a connector body having a strain relief portion and a connection portion;
the strain relief portion comprising:
a first strain relief member;
a second strain relief member;
a third strain relief member;
a first opening extending through the connector body and disposed between the first and second strain relief members;
a second opening extending through the connector body and disposed between the second and third strain relief members; and
the connection portion comprising two or more openings for receiving the actuation element such that the actuation element is attached to the connector body by a stitched connection.

6. The catheter assembly according to claim 5, wherein the strain relief portion comprises one or more alignment walls for maintaining the actuation element within the strain relief portion.

7. The catheter assembly of claim 4 wherein the connector comprises:
a body having a base portion, a plurality of projections extending from the base portion, and a plurality of gaps;
each projection comprising a neck portion and a head portion larger than the neck portion, the neck portion located between the base portion and the head portion; and
each gap located between at least two projections.

8. The catheter assembly of claim 7 wherein at least one of the neck portions and at least one of the head portions form a cleat.

9. The catheter assembly of claim 4 wherein the connector comprises:
an actuation element configured to flex a distal catheter end;
a first clamp half;
a second clamp half;
wherein the actuation element is clamped between the first clamp half and the second clamp half.

10. The catheter assembly of claim 9 wherein each clamp half includes a tortuous surface that bends the actuation element when the first clamp half is clamped to the second clamp half.

11. The catheter assembly of claim 1 further comprising a seal assembly that comprises:
a seal housing having one or more alignment members;
a seal member disposed within the seal housing; and
a cap configured to attach to the seal housing, the cap having one or more alignment slots that are configured to receive the one or more alignment members of the seal housing.

12. The seal assembly according to claim 11, wherein the seal housing comprises three alignment members, each alignment member being positioned 120 degrees from an adjacent alignment member; and
wherein the cap comprises six alignment slots, each alignment slot being positioned 60 degrees from an adjacent alignment slot.

13. The catheter assembly according to claim 11, wherein the cap comprises a plurality of connection members for attaching the cap to the seal housing and wherein the plurality of connection members are configured to attach the cap to the seal housing by a snap connection and wherein the seal housing comprises one or more alignment members and the cap comprises one or more alignment slots for receiving the alignment members of the seal housing.

14. The catheter assembly according to claim 1, further comprising a catheter coupler, the catheter coupler comprising:
a catheter connection lumen connected to the steerable catheter;
an outer circumferential passage;
a plurality of connecting passages that each connect the catheter connection lumen to the outer circumferential passage, wherein each of the connecting passages is offset from a center line of the catheter connection lumen; and
an outlet portion in fluid communication with the outer circumferential passage.

15. A catheter assembly for a transvascular deliver system comprising:
a patterned tube disposed between an inner and an outer liners, the patterned tube comprising a tube wall having at least first and second pattern openings;
the first pattern openings comprising an interrupted spiral pattern;
the second pattern openings comprising a plurality of flex windows and a plurality of reflow openings disposed opposite the flex windows; and
the plurality of flex windows comprising a transition region having flex windows of different size.

16. The catheter assembly according to claim 15 wherein the transition region is disposed proximate a plurality of flex windows having the same size.

17. The catheter assembly according to 15 wherein the flex windows of different size comprises at least a first flex window having a first size, a second flex window having a second size that is larger than the first size, and a third flex window having a third size that is larger than the second size.

18. A catheter assembly for a transvascular delivery system comprising:
a handle housing;
a sheath extending longitudinally from the handle housing;
wherein the sheath comprises a coil that includes one or more supporting elements and one or more coiled tubes;
wherein the one or more supporting elements and the one or more coiled tubes together define a main lumen; and
wherein the lumen of each of the one or more tubes is configured receive a control element such that the control element can be moved in a direction from a proximal portion of the sheath to a distal portion of the sheath.

19. The catheter assembly according to claim 18, wherein the one or more coiled tubes of the coil include at least two coiled tubes.

20. The catheter assembly according to claim 19, wherein a portion of the one or more coiled tubes is straight.
